(19) United States
(12) Patent Application Publication
(10) Pub. No.: US 2024/0150807 A1
(43) Pub. Date: May 9, 2024

(54) Method for Producing Cysteine Knot Protein

(71) Applicant: Sumitomo Pharma Co., Ltd., Osaka (JP)

(72) Inventors: Hidetaka NAGATA, Osaka-shi, Osaka (JP); WenLien LIN, Osaka-shi, Osaka (JP); Reiko ASADA, Osaka-shi, Osaka (JP); Kenji TAKIKAWA, Osaka-shi, Osaka (JP)

(73) Assignee: Sumitomo Pharma Co., Ltd., Osaka (JP)

(21) Appl. No.: 18/280,574

(22) PCT Filed: Mar. 9, 2022
(86) PCT No.: PCT/JP2022/010391

§ 371 Date: Sep. 6, 2023

(30) Foreign Application Priority Data
Mar. 10, 2021 (JP) 2021-038367

(51) Int. Cl. C12P 21/02; C12N 15/85

(52) U.S. Cl. CPC C12P 21/02; C12N 15/85

(57) ABSTRACT

A method for producing a cysteine knot protein, the method including: producing the cysteine knot protein by culturing a transformed mammalian cell containing a gene encoding the cysteine knot protein and a gene encoding an exogenous chaperone protein in a protein production medium; and collecting the produced cysteine knot protein, wherein the chaperone protein includes one or more selected from the group consisting of HSP90α, HSP90β, CDC37, HSP70, HSP40, HSP60, HSP10, HSP110, and HSP27.

### TECHNICAL FIELD

The present invention relates to a method for producing a cysteine knot protein.

### BACKGROUND ΔRT

Recombinant proteins are currently used in a wide range of fields. The importance is further increasing because of the recent development of biopharmaceuticals. Recombinant proteins are produced primarily with Escherichia coli, yeasts, insect cells, mammalian cells, or the like as host cells (e.g., Japanese National Patent Publication No. 2007-524381 (PTL 1)). Use of such host cells allows recombinant proteins to be given in large quantities in short times. However, recombinant proteins expressed may be incapable of exerting the original functions possessed by the recombinant proteins in some cases, for example, because the recombinant proteins do not correctly fold or have not undergone posttranslational modification (e.g., addition of a sugar chain) or the like.

In particular, difficult-to-express proteins (DEP) have been known to be difficult to produce as a recombinant protein in large quantities.

### CITATION LIST

### Patent Literature

PTL 1: Japanese National Patent Publication No. 2007-524381

### Non Patent Literature

NPL 1: Lee et al., Journal of Biotechnology 143 (2009) 34-43

### SUMMARY OF INVENTION

### Technical Problem

Among difficult-to-express proteins, a series of proteins called cysteine knot proteins have been attracting attention as proteins that can be materials for pharmaceuticals and the like, that is, active ingredients therefor (FIG. 1, FIG. 2), and development of a method that allows them to be produced in large quantities has been demanded.

The present invention has been made in view of such circumstances, and an object of the present invention is to provide a method for producing a cysteine knot protein with enhanced production efficiency.

### Solution to Problem

The present inventors diligently studied to achieve the object to find that co-expression of a gene encoding a cysteine knot protein and a gene encoding a specific chaperone protein in a mammalian cell as a host cell results in enhanced production efficiency for the cysteine knot protein, completing the present invention.

Specifically, the present invention is as follows.

[1] A method for producing a cysteine knot protein of the present invention includes:
producing the cysteine knot protein by culturing a transformed mammalian cell containing a gene encoding the cysteine knot protein and a gene encoding an exogenous chaperone protein in a protein production medium; and
collecting the produced cysteine knot protein, wherein
the chaperone protein includes one or more selected from the group consisting of HSP90α, HSP90β, CDC37, HSP70, HSP40, HSP60, HSP10, HSP110, and HSP27.

[2] It is preferable that the method for producing a cysteine knot protein of the present invention include:
providing a mammalian cell;
transforming the mammalian cell with a gene encoding the cysteine knot protein and a gene encoding the chaperone protein;
producing the cysteine knot protein by culturing the transformed mammalian cell in a protein production medium; and
collecting the produced cysteine knot protein, wherein
the chaperone protein includes one or more selected from the group consisting of HSP90α, HSP90β, CDC37, HSP70, HSP40, HSP60, HSP10, HSP110, and HSP27.

[3] It is preferable in the method for producing a cysteine knot protein according to [2] that the transforming the mammalian cell be performed with one or more recombinant protein expression vectors each containing a gene encoding the cysteine knot protein and a gene encoding the chaperone protein.

[4] It is preferable in the method for producing a cysteine knot protein according to [2] that the transforming the mammalian cell be performed by simultaneously or separately bringing one or more recombinant protein expression vectors each containing a gene encoding the cysteine knot protein and one or more expression-enhancing vectors each containing a gene encoding the chaperone protein into contact with the mammalian cell.

[5] It is preferable that the method for producing a cysteine knot protein of the present invention include:
providing a mammalian cell containing one or more recombinant protein expression vectors each containing a gene encoding the cysteine knot protein;
transforming the mammalian cell with at least one expression-enhancing vector containing a gene encoding the chaperone protein;
producing the cysteine knot protein by culturing the transformed mammalian cell in a protein production medium; and
collecting the produced cysteine knot protein, wherein
the chaperone protein includes one or more selected from the group consisting of HSP90α, HSP90β, CDC37, HSP70, HSP40, HSP60, HSP10, HSP110, and HSP27.

[6] It is preferable in the method for producing a cysteine knot protein according to [5] that
the expression-enhancing vector include a first expression-enhancing vector containing a gene encoding a first chaperone protein and a second expression-enhancing vector containing a gene encoding a second chaperone protein, and
the first chaperone protein be different from the second chaperone protein.

[7] It is preferable that the chaperone protein include either one or both of HSP90α and CDC37.

[8] It is preferable that
the cysteine knot protein have a cysteine knot motif having two or more cysteine residues, and
the two or more cysteine residues be forming one or more intramolecular disulfide bonds.

[9] It is preferable that the cysteine knot protein include one or more selected from the group consisting of neurotrophins, proteins belonging to the PDGF like super family, proteins belonging to the TGFβ super family, coagulogen, noggin, IL-17F, proteins belonging to the thyroid stimulating hormone family, and proteins belonging to the gonadotropic hormone family.

It is preferable that the cysteine knot protein include one or more selected from the group consisting of BDNF, NT3, PDGF-β, GDNF, IL-17F, and NGF.

It is preferable that the mammalian cell include one or more selected from the group consisting of a CHO cell, a COS cell, a BHK cell, a HeLa cell, an HEK293 cell, an NS0 cell, and an Sp2/0 cell.

A mammalian cell for recombinant protein production according to the present invention is a mammalian cell for recombinant protein production containing one or more recombinant protein expression vectors each containing a gene encoding a cysteine knot protein, wherein
the mammalian cell for recombinant protein production further contains one or more expression-enhancing vectors each containing a gene encoding a chaperone protein, and
the chaperone protein includes one or more selected from the group consisting of HSP90α, HSP90β, CDC37, HSP70, HSP40, HSP60, HSP10, HSP110, and HSP27.

[13] A kit according to the present invention is a kit for enhancing the production of a cysteine knot protein in a mammalian cell, wherein
the kit includes one or more expression-enhancing vectors each containing a gene encoding a chaperone protein, and
the chaperone protein includes at least one selected from the group consisting of HSP90α, HSP90β, CDC37, HSP70, HSP40, HSP60, HSP10, HSP110, and HSP27.

### Advantageous Effect of Invention

The present invention enables providing a method for producing a cysteine knot protein with enhanced production efficiency.

### BRIEF DESCRIPTION OF DRAWINGS

FIG. 1 is a schematic diagram illustrating the classification of difficult-to-express proteins.

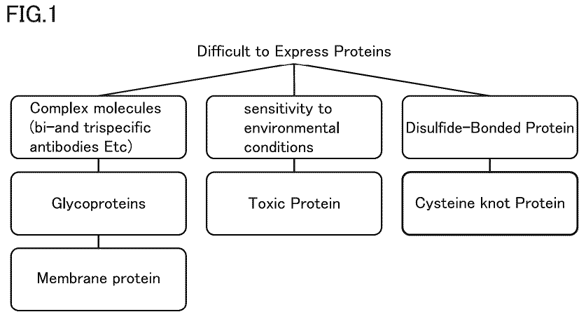

FIG. 2 is a schematic diagram illustrating the classification of proteins belonging to the cysteine knot protein super family.

### DESCRIPTION OF EMBODIMENTS

The following describes an embodiment of the present invention (hereinafter, occasionally expressed as “the present embodiment”). However, the present embodiment is not limited to the description. Herein, expressions in the format of “A to Z” each indicate the upper limit and lower limit of a range (i.e., A or more and Z or less). In the case that a unit is shown not for A but only for Z, the unit of A and the unit of Z are the same.

The method for producing a cysteine knot protein of the present embodiment includes:
producing the cysteine knot protein by culturing a transformed mammalian cell containing a gene encoding the cysteine knot protein and a gene encoding an exogenous chaperone protein in a protein production medium; and
collecting the produced cysteine knot protein, wherein
the chaperone protein includes one or more selected from the group consisting of HSP90α, HSP90β, CDC37, HSP70, HSP40, HSP60, HSP10, HSP110, and HSP27.

In an aspect of the present embodiment, the transformed mammalian cell can be obtained with a method including:
providing a mammalian cell containing one or more recombinant protein expression vectors each containing a gene encoding the cysteine knot protein; and
transforming the mammalian cell with at least one expression-enhancing vector containing a gene encoding a chaperone protein.

The details will be described in “Method for producing cysteine knot protein (1)” shown later.

In another aspect of the present embodiment, the transformed mammalian cell can be obtained with a method including:
providing a mammalian cell; and
transforming the mammalian cell with a gene encoding the cysteine knot protein and a gene encoding a chaperone protein.
   The details will be described in “Method for producing cysteine knot protein (2)” shown later.

<<Method for Producing Cysteine Knot Protein (1)>>

The first method for producing a cysteine knot protein of the present embodiment includes:
providing a mammalian cell containing one or more recombinant protein expression vectors each containing a gene encoding the cysteine knot protein;
transforming the mammalian cell with at least one expression-enhancing vector containing a gene encoding a chaperone protein;
producing the cysteine knot protein by culturing the transformed mammalian cell in a protein production medium; and
collecting the produced cysteine knot protein, wherein
the chaperone protein includes one or more selected from the group consisting of HSP90α, HSP90β, CDC37, HSP70, HSP40, HSP60, HSP10, HSP110, and HSP27. The following describes the first method in detail.

<Providing Mammalian Cell Containing Recombinant Protein Expression Vector>

In this step, a mammalian cell containing one or more recombinant protein expression vectors each containing a gene encoding a cysteine knot protein is provided.

(Cysteine Knot Protein)

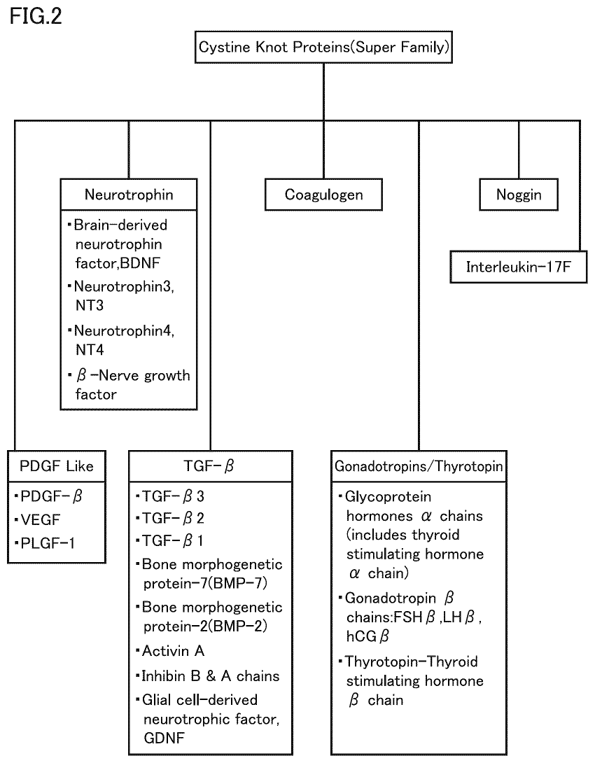

In the present embodiment, the term “cysteine knot protein” refers to a protein belonging to the cysteine knot protein super family (e.g., FIG. 2). It is preferable that the cysteine knot protein include one or more selected from the group consisting of neurotrophins, proteins belonging to the PDGF like super family (platelet-derived growth factor like super family), proteins belonging to the TGFβ super family (transforming growth factor (3 super family), coagulogen, noggin, IL-17F (interleukin-17F), proteins belonging to the thyroid stimulating hormone family, and proteins belonging to the gonadotropic hormone family.

The neurotrophins include brain-derived neurotrophic factor (BDNF), neurotrophin 3 (NT3), neurotrophin 4 (NT4), and nerve growth factors (NGF). Examples of NGF include β nerve growth factor (β-NGF).

Here, the BDNF is a known protein discovered by Barde et al. in 1982 and cloned by Jones et al. in 1990 (EMBO J, (1982) 1: 549-553, Proc. Natl. Acad. Sci. USA (1990) 87: 8060-8064). The term BDNF encompasses: a mature BDNF, which exerts the functions in vivo; a BDNF precursor, a premature form of BDNF (also referred to as “BDNF pro-form”); and a precursor of the BDNF precursor (also referred to as “BDNF pre-pro-form”), which is formed by adding a signal peptide to the N terminus of the BDNF precursor. Specifically, the BDNF is first formed as a BDNF pre-pro-form from the gene transcription product, and a signal peptide is cleaved from the BDNF pre-pro-form to leave a BDNF pro-form. Thereafter, an N-terminal amino acid sequence is cleaved from the BDNF pro-form to leave a mature BDNF.

The proteins belonging to the PDGF like super family include platelet-derived growth factor-β (PDGF-β), vascular endothelial growth factors (VEGF), and placental growth factor-1 (PLGF-1).

The proteins belonging to the TGFβ super family include transforming growth factor-β1 (TGF-β1), transforming growth factor-β2 (TGF-β2), transforming growth factor-β3 (TGF-β3), bone morphogenetic protein-2 (BMP-2), bone morphogenetic protein-7 (BMP-7), activin A, inhibin A, inhibin B, and glial cell line-derived neurotrophin (GDNF).

The proteins belonging to the thyroid stimulating hormone family include thyroid stimulating hormone α chain and thyrotropin (thyroid stimulating hormone β chain).

The proteins belonging to the gonadotropic hormone family include follicle stimulating hormone β chain (FSHβ), luteinizing hormone β chain (LHβ), and human chorionic gonadotropin (3 chain (hCGβ).

In the present embodiment, it is more preferable that the cysteine knot protein include one or more selected from the group consisting of BDNF, NT3, PDGF-β, GDNF, IL-17F, and NGF.

In an aspect of the present embodiment, the cysteine knot protein can be regarded as a protein having a cysteine knot motif having two or more cysteine residues. Here, it is preferable that the two or more cysteine residues be forming one or more intramolecular disulfide bonds. The term “cysteine knot motif” refers to any amino acid sequence having at least six cysteine residues and being capable of forming at least three disulfide bonds. Specifically, the cysteine knot motif contains six to eight cysteine residues capable of forming three disulfide bonds, eight or nine cysteine residues capable of forming four disulfide bonds, 10 cysteine residues capable of forming five disulfide bonds, or 12 cysteine residues capable of forming six disulfide bonds. The cysteine knot motif is preferably such that the number of amino acid residues contained between the cysteine residue in the N-terminal side of the protein of mature form, that is, active form and the cysteine residue in the C-terminal side thereof is 76 to 112. In the case of six cysteine residues with three disulfide bonds, for example, the cysteine knot motif has a sequence of Cys-X (42 to 59 amino acids)-Cys-X (4 to 16 amino acids)-Cys-X (11 to 29 amino acids)-Cys-X (one amino acid)-Cys. Here, X is any amino acid residue other than cysteine residues (the same applies hereinafter). In the case of seven cysteine residues with three disulfide bonds, the cysteine knot motif has a sequence of Cys-X (26 to 28 amino acids)-Cys-X (three amino acids)-Cys-X (28 to 31 amino acids)-Cys-Cys-X (28 to 31 amino acids)-Cys-X (one amino acid)-Cys. In the case of eight cysteine residues with three disulfide bonds, the cysteine knot motif has a sequence of Cys-X (25 or 26 amino acids)-Cys-X (five amino acids)-Cys-X (two amino acids)-Cys-Cys-X (six amino acids)-Cys-X (32 to 36 amino acids)-Cys-X (one amino acid)-Cys. In the case of eight cysteine residues with four disulfide bonds, the cysteine knot motif has a sequence of Cys-X (22 amino acids)-Cys-X (five amino acids)-Cys-X (seven amino acids)-Cys-X (14 amino acids)-Cys-X (seven amino acids)-Cys-X (12 amino acids)-Cys-X (one amino acid)-Cys. In the case of nine cysteine residues with four disulfide bonds, the cysteine knot motif has a sequence of Cys-X (six to nine amino acids)-Cys-Cys-X (26 or 27 amino acids)-Cys-X (three amino acids)-Cys-X (28 to 35 amino acids)-Cys-Cys-X (30 or 31 amino acids)-Cys-X (one amino acid)-Cys or Cys-X (24 amino acids)-Cys-X (24 amino acids)-Cys-X (five amino acids)-Cys-X (two amino acids)-Cys-Cys-X (six amino acids)-Cys-X (33 amino acids)-Cys-X (one amino acid)-Cys. In the case of 10 cysteine residues with five disulfide bonds, the cysteine knot motif has a sequence of Cys-X (two amino acids)-Cys-X (17 amino acids)-Cys-X (two amino acids)-Cys-Cys-X (25 amino acids)-Cys-Cys-X (21 amino acids)-Cys-X (one amino acid) Cys-X (two amino acids)-Cys. In the case of 12 cysteine residues with six disulfide bonds, the cysteine knot motif has a sequence of Cys-X (13 amino acids)-Cys-X (two amino acids)-Cys-X (seven amino acids)-Cys-X (three amino acids)-Cys-X (18 to 20 amino acids)-Cys-X (14 or 15 amino acids)-Cys-X (15 amino acids)-Cys-X (one amino acid)-Cys-X (two amino acids)-Cys-X (six amino acids)-Cys-X (nine amino acids)-Cys.

Checking whether a protein has a cysteine knot motif and whether correct disulfide bonds are formed in a cysteine knot protein produced with the method of the present invention is carried out, for example, as follows. First, a protein of interest (a protein having a cysteine knot motif) in a reduced state and the same protein in a non-reduced state are subjected to mass spectrometry (e.g., LC-MS or LC-MS/MS) to measure the molecular weight in the reduced state and that in the non-reduced state. From the measurement results obtained, the difference between molecular weight in a reduced state and that in a non-reduced state is determined for the protein of interest and fragments thereof; thereby the presence or absence of a disulfide bond in the protein of interest can be identified.

Whether correct disulfide bonds are formed in a cysteine knot protein produced with the method of the present invention can be tested by comparing the physiological activity of the protein with that of a standard product. Examples of the physiological activity of neurotrophins such as BDNF and NGF include phosphorylation of TrkA or TrkB, dimerization, and in vitro activity of downstream signals (MAPK cascade, CREB, etc.).

Among cysteine knot proteins, in particular, highly difficult-to-express proteins more significantly tend to benefit from the protein production level-increasing effect of the present invention. That is, an example of the cysteine knot protein in the present embodiment is a difficult-to-express cysteine knot protein, specifically, a difficult-to-express cysteine knot protein with low expression at a protein level (including posttranslational modification). Examples of the highly difficult-to-express cysteine knot protein include BDNF, NGF, GDNF, chorionic gonadotropin 13 chain, and glycoprotein hormone a chain.

Herein, the cysteine knot protein may be a fusion protein with an additional protein. In an aspect of the present embodiment, the fusion protein may consist only of a cysteine knot protein and an additional protein. In another aspect of the present embodiment, the fusion protein may be composed of a cysteine knot protein, an additional protein, and a linker peptide linking the cysteine knot protein and the additional protein. The linker peptide is not limited as long as it has a known amino acid sequence. Examples of the linker peptide include GS linkers, which are of flexible type, and H4 linkers, which are of rigid type. Examples of GS linkers include peptide linkers with one to eight repetitions of (Gly-Gly-Gly-Gly-Ser) (SEQ ID NO: 87). Examples of H4 linkers include peptide likers with two to four repetitions of (Glu-Ala-Ala-Ala-Ala-Lys) (SEQ ID NO: 88).

In the fusion protein, the cysteine knot protein and the additional protein may be disposed in the N-terminal side and in the C-terminal side, respectively. In the fusion protein, the additional protein and the cysteine knot protein may be disposed in the N-terminal side and in the C-terminal side, respectively.

Examples of the additional protein include antibodies, antibody fragments, and human serum albumin protein, and the additional protein may be a monomer, or a dimer composed of two subunits, or a multimer composed of multiple subunits. Examples of antibody fragments include a Fab fragment, which consists of a heavy chain (H chain) fragment of an antibody and a light chain (L chain) fragment of an antibody, an Fc fragment, which contains an antibody constant region, a single-chain antibody (scFv), and a bispecific antibody (diabody).

An example of the fusion protein is a fusion protein in which an Fc fragment is binding to the C terminus of a cysteine knot protein such as BDNF, GDNF, NGF, and IL17F via a peptide linker.

(Recombinant Protein Expression Vector)

In the present embodiment, the term “recombinant protein expression vector” refers to a DNA construct in which a gene encoding a target recombinant protein has been introduced in such a manner that the gene can be expressed in a host cell. The term “recombinant protein” refers to an exogenous protein for the host cell. In the present embodiment, the target recombinant protein is a cysteine knot protein. That is, the recombinant protein expression vector contains a gene encoding the cysteine knot protein.

In the case that the cysteine knot protein is a fusion protein with an additional protein and the additional protein is a dimer, the gene encoding the fusion protein may be composed of a first gene containing the nucleotide sequence of a gene encoding the cysteine knot protein and the nucleotide sequence of a gene encoding the first subunit constituting the additional protein, and a second gene containing the nucleotide sequence of a gene encoding the second subunit constituting the additional protein. In this case, the recombinant protein expression vector may be composed of a first recombinant protein expression vector containing the first gene and a second recombinant protein expression vector containing the second gene. Alternatively, the recombinant protein expression vector may contain both the first gene and the second gene. Even in the case that the additional protein is a multimer, design of the gene encoding the fusion protein and design of the recombinant protein expression vector can be carried out in the same manner as in the aforementioned case of dimer.

In the present embodiment, the nucleotide sequence of the gene encoding the cysteine knot protein may be a wild-type nucleotide sequence, and may be a nucleotide sequence formed by introducing one or more mutations into the wild-type nucleotide sequence as long as at least one cysteine knot motif is retained, or preferably 50% or more, more preferably 80% or more, 90% or more, or 100% of a cysteine knot motif is retained. Specifically, the nucleotide sequence of the gene encoding the cysteine knot protein may be:
(A) a nucleotide sequence having a sequence identity of 90% or more and 100% or less with the wild-type nucleotide sequence encoding the cysteine knot protein;
(B) a nucleotide sequence formed by deleting, substituting, inserting, or adding one or several nucleotides for the wild-type nucleotide sequence encoding the cysteine knot protein;
(C) a nucleotide sequence hybridizable with an oligonucleotide having a nucleotide sequence complementary to the wild-type nucleotide sequence encoding the cysteine knot protein under stringent conditions;
(D) a nucleotide sequence encoding an amino acid sequence having a sequence identity of 90% or more and 100% or less with the wild-type amino acid sequence of the cysteine knot protein; or
(E) a nucleotide sequence encoding an amino acid sequence formed by deleting, substituting, inserting, or adding one or several amino acid residues for the wild-type amino acid sequence of the cysteine knot protein, and
the nucleotide sequence of the gene encoding the cysteine knot protein may be a nucleotide sequence encoding a protein retaining the original functions of the cysteine knot protein.

In the present embodiment, “sequence identity” means the proportion (%) of identical nucleotides to the total nucleotides of an overlapping nucleotide sequence in optimum alignment given by aligning two nucleotide sequences with a mathematical algorithm known in the art (preferably, the algorithm allows introduction of a gap into one or both of sequences to be considered for optimum alignment). Those skilled in the art could check the “sequence identity” of a nucleotide sequence with ease. For example, NCBI BLAST (National Center for Biotechnology Information Basic Local Alignment Search Tool) can be used. The sequence identity of an amino acid sequence can also be checked with the same method as described.

The nucleotide sequence of the gene encoding the cysteine knot protein may have a sequence identity of 95% or more and 100% or less, or a sequence identity of 98% or more and 100% or less, or a sequence identity of 100% with the wild-type nucleotide sequence encoding the cysteine knot protein.

In the present embodiment, examples of the “nucleotide sequence formed by deleting, substituting, inserting, or adding one or several nucleotides” include a nucleotide sequence having a sequence identity of 80% or more, 85% or more, 90% or more, 95% or more, 97% or more, 98% or more, or 99% or more with the nucleotide sequence before deletion, substitution, insertion, or addition as a result of deletion, substitution, insertion, or addition. Regarding the specific number for “one or several nucleotides”, any one of such deletion, substitution, insertion, and addition may be present at one position, two positions, three positions, four positions, or five positions, and two or more of such deletion, substitution, insertion, and addition may occur in combination.

In the present embodiment, the term “stringent conditions” refers to conditions involving incubating in a solution containing 6×SSC (composition of 1×SSC: 0.15 M NaCl, 0.015 M sodium citrate, pH 7.0), 0.5% SDS, 5×Denhardt's solution, 100 μg/mL modified salmon sperm DNA, and 50% (v/v) formamide at room temperature for 12 hours, and further washing with 0.5×SSC at a temperature of 50° C. or more. Moreover, the term “stringent conditions” also encompasses more stringent conditions, for example, more severe conditions such as incubating at 45° C. or 60° C. for 12 hours, washing with 0.2×SSC or 0.1×SSC, and washing under a temperature condition of 60° C. or 65° C. or more in washing.

In an aspect of the present embodiment, the nucleotide sequence of the gene encoding the cysteine knot protein may be a nucleotide sequence subjected to optimization of codons with considering codon usage frequencies in the mammalian cell into which the gene is to be introduced. The optimization of codons is performed, for example, as follows. Specifically, the optimization of codons can be performed by using an algorithm capable of optimizing transcription, translational effects, and folding formation, as typified by Codon W (e.g., see http://codonw.sourceforge.net/index.html).

In an aspect of the present embodiment, in the case that the cysteine knot protein is a secretory protein, the concept of the cysteine knot protein includes both a protein containing a signal peptide and a protein with a signal peptide cleaved therefrom. Therefore, the nucleotide sequence of the gene encoding the cysteine knot protein may be a nucleotide sequence of a gene encoding a protein containing a signal peptide at the N terminus. The signal peptide can be one possessed by a natural form of the cysteine knot protein, and even can be replaced with a signal peptide in any protein. Examples include a signal peptide of human IL2 (Met-Tyr-Arg-Met-Gln-Leu-Leu-Ser-Cys-Ile-Ala-Leu-Ser-Leu-Ala-Leu-Val-Thr-Asn-Ser) (SEQ ID NO: 97) and a signal peptide of human albumin (Met-Lys-Trp-Val-Thr-Phe-Ile-Ser-Leu-Phe-Leu-Phe-Ser-Ser-Ala-Tyr-S er) (SEQ ID NO: 98).

In an aspect of the present embodiment, in the case that a precursor and a mature form are present for the cysteine knot protein, the concept of the cysteine knot protein includes both of them. Therefore, the nucleotide sequence of the gene encoding the cysteine knot protein may be a nucleotide sequence of a gene encoding the precursor protein, or a nucleotide sequence of a gene encoding the mature form protein.

In the present embodiment, examples of the nucleotide sequence of a gene encoding NGF include nucleotide sequences set forth in SEQ ID NO: 31 and SEQ ID NO: 71 (GenBank No. NM 002506, human-derived wild nucleotide sequences).

Examples of the nucleotide sequence of a gene encoding PDGF-β include nucleotide sequences set forth in SEQ ID NO: 33 and SEQ ID NO: 73 (GenBank No. NM_002608, human-derived wild nucleotide sequences).

Examples of the nucleotide sequence of a gene encoding IL-17F include nucleotide sequences set forth in SEQ ID NO: 35 and SEQ ID NO: 75 (GenBank No. NM_052872, human-derived wild nucleotide sequences).

Examples of the nucleotide sequence of a gene encoding GDNF include nucleotide sequences set forth in SEQ ID NO: 37 and SEQ ID NO: 77 (GenBank No. NM_000514, human-derived wild nucleotide sequences).

Examples of the nucleotide sequence of a gene encoding NT3 include nucleotide sequences set forth in SEQ ID NO: 39 and SEQ ID NO: 79 (GenBank No. NM_002527, human-derived wild nucleotide sequences).

Examples of the nucleotide sequence of a gene encoding BDNF include a nucleotide sequence encoding the BDNF pre-pro-form described above, a nucleotide sequence encoding the BDNF pro-form, and a nucleotide sequence encoding the mature BDNF. Examples of the nucleotide sequence encoding the BDNF pre-pro-form include nucleotide sequences set forth in SEQ ID NO: 43 and SEQ ID NO: 83 (GenBank No. NM_170735.6, human-derived wild nucleotide sequences). Examples of the nucleotide sequence encoding the BDNF pro-form include a nucleotide sequence encoding the amino acid sequence of a BDNF pro-form formed by removing a signal peptide corresponding to the 18 N-terminal amino acid residues of the BDNF pre-pro-form (e.g., SEQ ID NO: 89). Examples of the nucleotide sequence encoding the mature BDNF include a nucleotide sequence encoding a mature BDNF formed by removing the 110 N-terminal amino acid residues of the BDNF pro-form (e.g., SEQ ID NO: 91).

Examples of the nucleotide sequence of a gene encoding a fusion protein of BDNF and an Fc fragment include a nucleotide sequence set forth in SEQ ID NO: 85.

Examples of the nucleotide sequence of a gene encoding a fusion protein of GDNF and an Fc fragment include a nucleotide sequence set forth in SEQ ID NO: 99.

The amino acid sequence of the cysteine knot protein may have a sequence identity of 95% or more and 100% or less, or a sequence identity of 98% or more and 100% or less, or a sequence identity of 100% with the wild-type amino acid sequence of the cysteine knot protein.

In the present embodiment, examples of the “amino acid sequence formed by deleting, substituting, inserting, or adding one or several amino acid residues” include an amino acid sequence having a sequence identity of 80% or more, 85% or more, 90% or more, 95% or more, 97% or more, 98% or more, or 99% or more with the amino acid sequence before deletion, substitution, insertion, or addition as a result of deletion, substitution, insertion, or addition. Regarding the specific number for “one or several amino acid residues”, any one of such deletion, substitution, insertion, and addition may be present at one position, two positions, three positions, four positions, or five positions, and two or more of such deletion, substitution, insertion, and addition may occur in combination.

In the present embodiment, examples of the amino acid sequence of NGF include an amino acid sequence set forth in SEQ ID NO: 32 (GenBank No. NP_002497). Examples of the amino acid sequence of PDGF-β include an amino acid sequence set forth in SEQ ID NO: 34 (GenBank No. NP_002599). Examples of the amino acid sequence of IL-17F include an amino acid sequence set forth in SEQ ID NO: 36 (GenBank No. NP_443104). Examples of the amino acid sequence of GDNF include an amino acid sequence set forth in SEQ ID NO: 38 (GenBank No. NP_000505). Examples of the amino acid sequence of NT3 include an amino acid sequence set forth in SEQ ID NO: 40 (GenBank No. NP_002518). Examples of the amino acid sequence of BDNF include the amino acid sequence of the BDNF pre-pro-form described above, the amino acid sequence of the BDNF pro-form, and the amino acid sequence of the mature BDNF. Examples of the amino acid sequence of the BDNF pre-pro-form include an amino acid sequence set forth in SEQ ID NO: 44 (GenBank No. NP_733931). Examples of the amino acid sequence of the BDNF pro-form include an amino acid sequence formed by removing the N-terminal signal peptide of the BDNF pre-pro-form (e.g., SEQ ID NO: 90). Examples of the amino acid sequence of the mature BDNF include an amino acid sequence formed by removing the 110 N-terminal amino acid residues of the BDNF pro-form (e.g., SEQ ID NO: 92). Here, the signal peptide in the BDNF pre-pro-form may be a signal peptide possessed by the wild BDNF pre-pro-form, or a signal peptide derived from another protein (e.g., a signal peptide consisting of an amino acid sequence set forth in SEQ ID NO: 94).

In the present embodiment, the nucleotide sequence of the gene encoding the additional protein may be a wild-type nucleotide sequence, or a nucleotide sequence formed by introducing one or more mutations into the wild-type nucleotide sequence. Specifically, the nucleotide sequence of the gene encoding the additional protein may be:
(A) a nucleotide sequence having a sequence identity of 90% or more and 100% or less with the wild-type nucleotide sequence encoding the additional protein;
(B) a nucleotide sequence formed by deleting, substituting, inserting, or adding one or several nucleotides for the wild-type nucleotide sequence encoding the additional protein;
(C) a nucleotide sequence hybridizable with an oligonucleotide having a nucleotide sequence complementary to the wild-type nucleotide sequence encoding the additional protein under stringent conditions;
(D) a nucleotide sequence encoding an amino acid sequence having a sequence identity of 90% or more and 100% or less with the wild-type amino acid sequence of the additional protein; or
(E) a nucleotide sequence encoding an amino acid sequence formed by deleting, substituting, inserting, or adding one or several amino acid residues for the wild-type amino acid sequence of the additional protein, and
the nucleotide sequence of the gene encoding the additional protein may be a nucleotide sequence encoding a protein retaining the original functions of the additional protein.

In the case that the additional protein is a dimer or a multimer, the aforementioned matters are applied to each of the genes encoding the subunits constituting the additional protein.

The nucleotide sequence of the gene encoding the additional protein may have a sequence identity of 95% or more and 100% or less, or a sequence identity of 98% or more and 100% or less, or a sequence identity of 100% with the wild-type nucleotide sequence encoding the additional protein.

In an aspect of the present embodiment, the nucleotide sequence of the gene encoding the additional protein may be a nucleotide sequence subjected to optimization of codons with considering codon usage frequencies in the mammalian cell into which the gene is to be introduced. The optimization of codons is performed, for example, with the method described above.

In the present embodiment, in the case that the additional protein is an Fc fragment, examples of the nucleotide sequence of a gene encoding the Fc fragment (first subunit and second subunit) of an antibody include a nucleotide sequence set forth in SEQ ID NO: 95 (GenBank No. JN222933).

The amino acid sequence of the additional protein may have a sequence identity of 95% or more and 100% or less, or a sequence identity of 98% or more and 100% or less, or a sequence identity of 100% with the wild-type amino acid sequence of the additional protein.

In the present embodiment, in the case that the additional protein is an Fc fragment, examples of the amino acid sequence of the Fc fragment (first subunit and second subunit) of an antibody include an amino acid sequence set forth in SEQ ID NO: 96 (GenBank No. AEV43323).

Examples of the amino acid sequence of a fusion protein of BDNF and an Fc fragment include an amino acid sequence set forth in SEQ ID NO: 86.

Examples of the amino acid sequence of a fusion protein of GDNF and an Fc fragment include an amino acid sequence set forth in SEQ ID NO: 100.

The recombinant protein expression vector contains not only the gene encoding the cysteine knot protein but also a promoter sequence (e.g., a cytomegalovirus (CMV) promoter, a thymidine kinase (TK) promoter of herpes simplex virus (HSV), an SV40 promoter, an EF-1 promoter, an actin promoter, a(3 globulin promoter, and an enhancer), a Kozak sequence, a terminator sequence, and an mRNA-stabilizing sequence. In an aspect of the present embodiment, the recombinant protein expression vector may further contain one or more selected from the group consisting of an origin of replication, an enhancer sequence, a signal sequence, a selection marker gene such as a drug resistance gene (e.g., a resistance gene against a drug such as ampicillin, tetracycline, kanamycin, chloramphenicol, neomycin, hygromycin, puromycin, and Zeocin), and a gene encoding a fluorescent protein such as GFP.

The recombinant protein expression vector is not limited as long as the advantageous effects of the present invention are exerted, and may be, for example, a plasmid vector or a viral vector. In an aspect of the present embodiment, it is preferable that the recombinant protein expression vector be a plasmid vector.

Examples of the plasmid vector include a pcDNA3.1(+) vector, a pcDNA3.3 vector, a pEGF-BOS vector, a pEF vector, a pCDM8 vector, a pCXN vector, a pCI vector, an episomal vector, and a transposon vector. In an aspect of the present embodiment, it is preferable that the plasmid vector be a pcDNA3.1(+) vector.

Examples of the viral vector include a lentiviral vector, an adenoviral vector, an adeno-associated virus vector, a Sendai virus vector, and a mammalian expression baculoviral vector. More specific examples include pLenti4/V5-GW/lacZ, pLVSIN-CMV, pLVSIN-EF1α, pAxcwit2, pAxEFwit2, pAAV-RCS, a pSeV vector, pFastBacMam, and pFastBacMam2.0 (VSV-G).

(Mammalian Cell)

In the present embodiment, the term “mammalian cell” refers to a cell derived from a mammal. Examples of the mammal include a human, a hamster (e.g., a Chinese hamster), a mouse, a rat, and a green monkey. The mammalian cell may be an immortalized cell.

The mammalian cell is not limited as long as it is used as a host cell for expression of the recombinant protein. Examples of such a mammalian cell include a CHO cell (a cell line derived from the ovary of a Chinese hamster), a COS cell (a cell line derived from the kidney of an African green monkey), a BHK cell (a cell line derived from the kidney of a baby hamster), a HeLa cell (a cell line derived from cervical cancer of a human), an HEK293 cell (a cell line derived from the kidney of a human embryo), an NS0 cell (a cell line derived from myeloma of a mouse), and an Sp2/0 cell (a cell line derived from myeloma of a mouse). Specifically, it is preferable that the mammalian cell include one or more selected from the group consisting of a CHO cell, a COS cell, a BHK cell, a HeLa cell, an HEK293 cell, an NS0 cell, and an Sp2/0 cell.

<Transforming Mammalian Cell with Expression-Enhancing Vector>

In this step, the mammalian cell is transformed with at least one expression-enhancing vector containing a gene encoding a chaperone protein.

(Chaperone Protein)

In the present embodiment, the term “chaperone protein” refers to a protein that assists the cysteine knot protein in correctly folding to attain the original functions. The chaperone protein includes one or more selected from the group consisting of HSP90α, HSP90β, CDC37 (Cell Division Cycle 37, HSP90 cochaperone), HSP70, HSP40, HSP60, HSP10, HSP110, and HSP27. Here, “HSP” is an abbreviation of heat shock protein. In an aspect of the present embodiment, it is preferable that the chaperone protein include any one of HSP90α, HSP90β, HSP40, and CDC37, or include both HSP90α, HSP90β, or HSP40 and CDC37. In another aspect of the present embodiment, it is preferable that the chaperone protein include either one or both of HSP90α and CDC37.

In an aspect of the present mode of implementation, the animal species from which the chaperone protein is derived may be the same as or different from the animal species from which the cysteine knot protein is derived.

In an aspect of the present mode of implementation, the animal species from which the chaperone protein is derived may be the same as or different from the animal species from which the host cell is derived.

In an aspect of the present mode of implementation, it is preferable that the animal species from which the chaperone protein is derived be the same as either one of the animal species from which the cysteine knot protein is derived or the animal species from which the host cell is derived.

In an aspect of the present embodiment, the chaperone protein may be a human-derived chaperone protein, or a Chinese hamster-derived chaperone protein. The chaperone protein may be preferably a human-derived chaperone protein.

In an aspect of the present embodiment, in the case that the cysteine knot protein is NGF, it is preferable that the chaperone protein include one or more selected from the group consisting of HSP90α, HSP90β, Chinese hamster-derived CDC37, HSP60, HSP110, and HSP27.

In an aspect of the present embodiment, in the case that the cysteine knot protein is NT3, it is preferable that the chaperone protein include one or more selected from the group consisting of HSP90α, HSP90β, CDC37, HSP70, HSP60, HSP10, HSP110, and HSP27.

In an aspect of the present embodiment, in the case that the cysteine knot protein is IL-17F, it is preferable that the chaperone protein include one or more selected from the group consisting of HSP90α, HSP90β, CDC37, HSP70, HSP60, HSP10, HSP110, and HSP27.

In an aspect of the present embodiment, in the case that the cysteine knot protein is PDGF-β, it is preferable that the chaperone protein include one or more selected from the group consisting of Chinese hamster-derived CDC37, HSP70, and HSP27.

In an aspect of the present embodiment, in the case that the cysteine knot protein is GDNF, it is preferable that the chaperone protein include one or more selected from the group consisting of Chinese hamster-derived HSP90α, human-derived HSP90β, HSP10, HSP70, and HSP27.

(Expression-Enhancing Vector)

In the present embodiment, the term “expression-enhancing vector” refers to a DNA construct in which a gene encoding the chaperone protein has been introduced in such a manner that the gene can be expressed in a host cell.

In the present embodiment, the nucleotide sequence of the gene encoding the chaperone protein may be a wild-type nucleotide sequence, or a nucleotide sequence formed by introducing one or more mutations into the wild-type nucleotide sequence. Specifically, the nucleotide sequence of the gene encoding the chaperone protein may be:
(A) a nucleotide sequence having a sequence identity of 90% or more and 100% or less with the wild-type nucleotide sequence encoding the chaperone protein;
(B) a nucleotide sequence formed by deleting, substituting, inserting, or adding one or several nucleotides for the wild-type nucleotide sequence encoding the chaperone protein;
(C) a nucleotide sequence hybridizable with an oligonucleotide having a nucleotide sequence complementary to the wild-type nucleotide sequence encoding the chaperone protein under stringent conditions;
(D) a nucleotide sequence encoding an amino acid sequence having a sequence identity of 90% or more and 100% or less with the wild-type amino acid sequence of the chaperone protein; or
(E) a nucleotide sequence encoding an amino acid sequence formed by deleting, substituting, inserting, or adding one or several amino acid residues for the wild-type amino acid sequence of the chaperone protein, and
the nucleotide sequence of the gene encoding the chaperone protein may be a nucleotide sequence encoding a protein that assists the cysteine knot protein in correctly folding to attain the original functions.

The nucleotide sequence of the gene encoding the chaperone protein may have a sequence identity of 95% or more and 100% or less, or a sequence identity of 98% or more and 100% or less, or a sequence identity of 100% with the wild-type nucleotide sequence encoding the chaperone protein.

In an aspect of the present embodiment, the nucleotide sequence of the gene encoding the chaperone protein may be a nucleotide sequence subjected to optimization of codons with considering codon usage frequencies in the mammalian cell into which the gene is to be introduced. The optimization of codons is performed, for example, with the method described above.

In the present embodiment, examples of the nucleotide sequence of a gene encoding HSP90α include nucleotide sequences set forth in SEQ ID NO: 45 (GenBank No. NM_001017963, human-derived wild nucleotide sequence), SEQ ID NO: 47 (GenBank No. NM_005348, human-derived wild nucleotide sequence), SEQ ID NO: 1, SEQ ID NO: 3, SEQ ID NO: 49 (GenBank No. NM_001246821, Chinese hamster-derived wild nucleotide sequence), and SEQ ID NO: 5.

Examples of the nucleotide sequence of a gene encoding HSP9013 include nucleotide sequences set forth in SEQ ID NO: 53 (GenBank No. NM_001271970, human-derived wild nucleotide sequence), SEQ ID NO: 55 (GenBank No.

NM_001271971, human-derived wild nucleotide sequence), SEQ ID NO: 51 (GenBank No. NM_001271972, human-derived wild nucleotide sequence), SEQ ID NO: 7, SEQ ID NO: 9, SEQ ID NO: 11, SEQ ID NO: 57 (GenBank No. XM_003501668.2, Chinese hamster-derived wild nucleotide sequence), and SEQ ID NO: 13.

Examples of the nucleotide sequence of a gene encoding CDC37 include nucleotide sequences set forth in SEQ ID NO: 59 (GenBank No. NM_007065, human-derived wild nucleotide sequence), SEQ ID NO: 15, SEQ ID NO: 61 (GenBank No. XM_003499737, Chinese hamster-derived wild nucleotide sequence), and SEQ ID NO: 17.

Examples of the nucleotide sequence of a gene encoding HSP60 include nucleotide sequences set forth in SEQ ID NO: 63 (GenBank No. NM_199440, human-derived wild nucleotide sequence) and SEQ ID NO: 19.

Examples of the nucleotide sequence of a gene encoding HSP40 include nucleotide sequences set forth in SEQ ID NO: 65 (GenBank No. NM_001539, human-derived wild nucleotide sequence) and SEQ ID NO: 21.

Examples of the nucleotide sequence of a gene encoding HSP10 include nucleotide sequences set forth in SEQ ID NO: 67 (GenBank No. NM_002157, human-derived wild nucleotide sequence) and SEQ ID NO: 23.

Examples of the nucleotide sequence of a gene encoding HSP110 include nucleotide sequences set forth in SEQ ID NO: 69 (GenBank No. NM_006644, human-derived wild nucleotide sequence) and SEQ ID NO: 25.

Examples of the nucleotide sequence of a gene encoding HSP70 include a CHO-derived wild nucleotide sequence described in Journal of Biotechnology 143 (2009) 34-43 and a nucleotide sequence set forth in SEQ ID NO: 27.

Examples of the nucleotide sequence of a gene encoding HSP27 include a CHO-derived wild nucleotide sequence described in Journal of Biotechnology 143 (2009) 34-43 and a nucleotide sequence set forth in SEQ ID NO: 29.

The amino acid sequence of the chaperone protein may have a sequence identity of 95% or more and 100% or less, or a sequence identity of 98% or more and 100% or less, or a sequence identity of 100% with the wild-type amino acid sequence of the chaperone protein.

In the present embodiment, examples of the amino acid sequence of HSP90α include amino acid sequences set forth in SEQ ID NO: 2 (GenBank No. NP_001017963), SEQ ID NO: 4 (GenBank No. NP_005339), and SEQ ID NO: 6 (GenBank No. NP_001233750).

Examples of the amino acid sequence of HSP9013 include amino acid sequences set forth in SEQ ID NO: 8 (GenBank No. NP_001258899), SEQ ID NO: 10 (GenBank No. NP_001258900), SEQ ID NO: 12 (GenBank No. NP_001258901), and SEQ ID NO: 14 (GenBank No. XP 003501716).

Examples of the amino acid sequence of CDC37 include amino acid sequences set forth in SEQ ID NO: 16 (Genbank No. NP_008996) and SEQ ID NO: 18 (GenBank No. XP 003499785).

Examples of the amino acid sequence of HSP60 include an amino acid sequence set forth in SEQ ID NO: 20 (GenBank No. NP_955472).

Examples of the amino acid sequence of HSP40 include an amino acid sequence set forth in SEQ ID NO: 22 (GenBank No. NP_001530).

Examples of the amino acid sequence of HSP10 include an amino acid sequence set forth in SEQ ID NO: 24 (GenBank No. NP_002148).

Examples of the amino acid sequence of HSP110 include an amino acid sequence set forth in SEQ ID NO: 26 (GenBank No. NP_006635).

Examples of the amino acid sequence of HSP70 include an amino acid sequence set forth in SEQ ID NO: 28 (described in Journal of Biotechnology 143 (2009) 34-43).

Examples of the amino acid sequence of HSP27 include an amino acid sequence set forth in SEQ ID NO: 30 (described in Journal of Biotechnology 143 (2009) 34-43).

The expression-enhancing vector contains not only the gene encoding the chaperone protein but also a promoter sequence (e.g., a cytomegalovirus (CMV) promoter, a thymidine kinase (TK) promoter of herpes simplex virus (HSV), an SV40 promoter, an EF-1 promoter, an actin promoter, a(3 globulin promoter, and an enhancer), a Kozak sequence, a terminator sequence, and an mRNA-stabilizing sequence. In an aspect of the present embodiment, the expression-enhancing vector may further contain one or more selected from the group consisting of an origin of replication, an enhancer sequence, a signal sequence, a selection marker gene such as a drug resistance gene (e.g., a resistance gene against a drug such as ampicillin, tetracycline, kanamycin, chloramphenicol, neomycin, hygromycin, puromycin, and Zeocin), and a gene encoding a fluorescent protein such as GFP.

The expression-enhancing vector is not limited as long as the advantageous effects of the present invention are exerted, and may be, for example, a plasmid vector or a viral vector. In an aspect of the present embodiment, it is preferable that the expression-enhancing vector be a plasmid vector.

Examples of the plasmid vector include a pcDNA3.1(+) vector, a pEGF-BOS vector, a pEF vector, a pCDM8 vector, a pCXN vector, a pCI vector, an episomal vector, and a transposon vector. In an aspect of the present embodiment, it is preferable that the plasmid vector be a pcDNA3.1(+) vector.

Examples of the viral vector include a lentiviral vector, an adenoviral vector, an adeno-associated virus vector, a Sendai virus vector, and a mammalian expression baculoviral vector. More specific examples include pLenti4/V5-GW/lacZ, pLVSIN-CMV, pLVSIN-EF1α, pAxcwit2, pAxEFwit2, pAAV-RCS, a pSeV vector, pFastBacMam, and pFastBacMam2.0 (VSV-G).

In the present embodiment, acquisition of a gene fragment encoding the cysteine knot protein, acquisition of a gene fragment encoding the chaperone protein, acquisition of a gene fragment encoding the additional protein, and construction of the plasmid vector can be carried out according to techniques conventionally used in the fields of molecular biology, biotechnology, and genetic engineering (e.g., Sambrook et al. “Molecular Cloning-A Laboratory Manual, second edition 1989”). Examples of the host cell to be used for preparing the plasmid vector include Escherichia coli, which is commonly used in the art.

In the present step, the mammalian cell can be suitably transformed with at least one expression-enhancing vector, whereas the mammalian cell may be transformed with a plurality of expression-enhancing vectors. Here, the mammalian cell is a mammalian cell containing one or more recombinant protein expression vectors each containing a gene encoding the cysteine knot protein, provided in the previous step. That is, in an aspect of the present embodiment, it is preferable that the expression-enhancing vector include a first expression-enhancing vector containing a gene encoding a first chaperone protein and a second expression-enhancing vector containing a gene encoding a second chaperone protein, and the first chaperone protein be different from the second chaperone protein. In this case, it is more preferable that the first chaperone protein be HSP90α and the second chaperone protein be CDC37.

Alternatively, the mammalian cell may be transformed with an expression-enhancing vector containing two or more genes each encoding a chaperone protein.

(Transformation with Expression-Enhancing Vector)

In the present embodiment, any known method can be used for transforming with the expression-enhancing vector without limitation as long as the advantageous effects of the present invention are exerted (e.g., Sambrook et al. “Molecular Cloning-A Laboratory Manual, second edition 1989”). Examples of known transformation methods include a lipofection method, a calcium phosphate method, a DEAE dextran method, an electroporation method, a polyethyleneimine method, and a polyethylene glycol method. The transformation may be performed with commercially available kit. Examples of such kits include a Gibco™ Expi™ Expression System (Cat. No. A29133) manufactured by ThermoFisher Scientific K.K.

In the case of the lipofection method, it is preferable to use 3 μg to 30 μg of an expression vector for a cell density of 1×10⁶ cells/mL to 9×10⁶ cells/mL. For example, it is preferable to use 20 μg in total of the expression-enhancing vector for cells (6×10⁶ cells/mL) contained in a 25-mL container.

<Producing Cysteine Knot Protein>

In this step, the cysteine knot protein is produced by culturing the transformed mammalian cell in a protein production medium.

Methods for producing recombinant proteins in large quantities by using Escherichia coli as host cells have been known so far; however, a method for producing a cysteine knot protein, which is a difficult-to-express protein, in large quantities as a soluble fraction such that the conformation that exhibits the functions is retained, by using mammalian cells as host cells has not been known yet. The method for producing a cysteine knot protein according to the present embodiment gives enhanced production efficiency for the cysteine knot protein by co-expressing a gene encoding the cysteine knot protein and a gene encoding the specific chaperone protein in the mammalian cell.

In culturing the transformed mammalian cell, the composition of the medium, the pH of the medium, the glucose concentration, the culture temperature, and the culture time, and other conditions such as the amounts of usage and time of usage of expression-inducing factors are appropriately adjusted so that the cysteine knot protein and the chaperone protein can be efficiently expressed.

The protein production medium to be used for culturing the transformed mammalian cell is not limited as long as it is a known medium suitable for protein production, and may be a solid medium or a liquid medium. It is preferable that the protein production medium be a liquid medium. Examples of the protein production medium include Dulbecco's modified Eagle medium (DMEM), Eagle minimal essential medium (MEM), Roswell Park Memorial Institute medium 1640 (RPMI1640), Iscove's modified Dulbecco's medium (IMDM), F10 medium, F12 medium, DMEM/F12, FreeStyle 293 expression medium, and Freestyle CHO medium. The protein production medium may contain fetal calf serum (FCS). The protein production medium may be a serum-free medium.

<Collecting Cysteine Knot Protein>

In this step, the produced cysteine knot protein is collected. The present step includes collecting the produced cysteine knot protein from the culture supernatant after the completion of culture. For example, after the completion of culture, the cysteine knot protein purified to have high purity can be obtained by treating the resulting culture supernatant with various purification methods.

At least one may be selected as a purification method from, for example, heat treatment and salting-out for the culture supernatant, and various chromatographies including anion-exchange chromatography, gel filtration chromatography, hydrophobic chromatography, hydroxyapatite chromatography, and affinity chromatography.

<<Method for Producing Cysteine Knot Protein (2)>>

A second method for producing a cysteine knot protein of the present embodiment includes:
providing a mammalian cell;
transforming the mammalian cell with a gene encoding the cysteine knot protein and a gene encoding a chaperone protein;
producing the cysteine knot protein by culturing the transformed mammalian cell in a protein production medium; and
collecting the produced cysteine knot protein, wherein
the chaperone protein includes one or more selected from the group consisting of HSP90α, HSP90β, CDC37, HSP70, HSP40, HSP60, HSP10, HSP110, and HSP27.

<Providing Mammalian Cell>

In this step, a mammalian cell is provided. For the mammalian cell, any of the mammalian cells shown as examples in “Method for producing cysteine knot protein (1)” in the above can be used. That is, it is preferable that the mammalian cell include one or more selected from the group consisting of a CHO cell, a COS cell, a BHK cell, a HeLa cell, an HEK293 cell, an NS0 cell, and an Sp2/0 cell.

<Transforming Mammalian Cell>

In this step, the mammalian cell is transformed with a gene encoding a cysteine knot protein and a gene encoding a chaperone protein.

For the cysteine knot protein, any of the cysteine knot proteins shown as examples in “Method for producing cysteine knot protein (1)” in the above can be used. That is, it is preferable that the cysteine knot protein include one or more selected from the group consisting of neurotrophins, proteins belonging to the PDGF like super family, proteins belonging to the TGFβ super family, coagulogen, noggin, IL-17F, proteins belonging to the thyroid stimulating hormone family, and proteins belonging to the gonadotropic hormone family. It is more preferable that the cysteine knot protein include one or more selected from the group consisting of BDNF, NT3, PDGF-β, GDNF, IL-17F, and NGF. The cysteine knot protein may be a fusion protein with an additional protein. In an aspect of the present embodiment, the fusion protein may consist only of a cysteine knot protein and an additional protein. In another aspect of the present embodiment, the fusion protein may be composed of a cysteine knot protein, an additional protein, and a linker peptide linking the cysteine knot protein and the additional protein.

For the chaperone protein, any of the chaperone proteins shown as examples in “Method for producing cysteine knot protein (1)” in the above can be used. That is, the chaperone protein includes one or more selected from the group consisting of HSP90α, HSP90β, CDC37, HSP70, HSP40, HSP60, HSP10, HSP110, and HSP27. In an aspect of the present embodiment, it is preferable that the chaperone protein include any one of HSP90α, HSP90β, HSP40, and CDC37, or include both HSP90α, HSP90β, or HSP40 and CDC37. In another aspect of the present embodiment, it is preferable that the chaperone protein include either one or both of HSP90α and CDC37.

In the present embodiment, the order of introducing the gene encoding the cysteine knot protein and the gene encoding the chaperone protein into the mammalian cell as a host cell is not limited. The gene encoding the cysteine knot protein and then the gene encoding the chaperone protein may be introduced into the mammalian cell. The gene encoding the chaperone protein and then the gene encoding the cysteine knot protein may be introduced into the mammalian cell. Alternatively, the gene encoding the cysteine knot protein and the gene encoding the chaperone protein may be simultaneously introduced into the mammalian cell.

For example, the ratio of the gene encoding the cysteine knot protein and the gene encoding the chaperone protein in introducing the two genes into the host cell may be 1:1 to 10:1, and preferably 3:1 to 5:1.

In an aspect of the present embodiment, it is preferable that the transforming the mammalian cell is performed with one or more recombinant protein expression vectors each containing a gene encoding the cysteine knot protein and a gene encoding the chaperone protein. Since the recombinant protein expression vectors each contain a gene encoding the cysteine knot protein together with a gene encoding the chaperone protein, the recombinant protein expression vectors can be regarded as expression-enhancing vectors.

In this case, in each of the recombinant protein expression vectors, identical or different promoter sequences may be disposed in the upstream of the gene encoding the cysteine knot protein and in the upstream of the gene encoding the chaperone protein. Construction of each recombinant protein expression vector in this manner allows expressions of the cysteine knot protein and the chaperone protein to be individually controlled.

In another aspect of the present embodiment, in each of the recombinant protein expression vectors, a promoter sequence, the gene encoding the cysteine knot protein, and the gene encoding the chaperone protein may be disposed in order from the 5′-end side, and a promoter sequence, the gene encoding the chaperone protein, and the gene encoding the cysteine knot protein may be disposed in order from the 5′-end side. Construction of each recombinant protein expression vector in this manner allows expressions of the cysteine knot protein and the chaperone protein to be simultaneously controlled with a single promoter sequence. The polypeptides expressed are inferred to be cleaved at proper sites to become the cysteine knot protein and the chaperone protein.

In another aspect of the present embodiment, it is preferable that the transforming the mammalian cell is performed by simultaneously or separately bringing one or more recombinant protein expression vectors each containing a gene encoding the cysteine knot protein and one or more expression-enhancing vectors each containing a gene encoding the chaperone protein into contact with the mammalian cell.

<Producing Cysteine Knot Protein>

In this step, the cysteine knot protein is produced by culturing the transformed mammalian cell in a protein production medium. For the specific method, the method described in “Method for producing cysteine knot protein (1)” in the above can be used.

<Collecting Cysteine Knot Protein>

In this step, the produced cysteine knot protein is collected. For the specific method, the method described in “Method for producing cysteine knot protein (1)” in the above can be used.

<<Mammalian Cell for Recombinant Protein Production>>

A mammalian cell for recombinant protein production in the present embodiment is a mammalian cell for recombinant protein production containing one or more recombinant protein expression vectors each containing a gene encoding a cysteine knot protein, wherein
the mammalian cell for recombinant protein production further contains one or more expression-enhancing vectors each containing a gene encoding a chaperone protein, and
the chaperone protein includes one or more selected from the group consisting of HSP90α, HSP90β, CDC37, HSP70, HSP40, HSP60, HSP10, HSP110, and HSP27.

<<Kit for Enhancing Production of Cysteine Knot Protein>>

A kit in the present embodiment is a kit for enhancing the production of a cysteine knot protein in a mammalian cell, wherein
the kit includes one or more expression-enhancing vectors each containing a gene encoding a chaperone protein, and
the chaperone protein includes at least one selected from the group consisting of HSP90α, HSP90β, CDC37, HSP70, HSP40, HSP60, HSP10, HSP110, and HSP27.

In the present embodiment, the kit may further include one or more selected from the group consisting of a buffer solution, a mammalian cell as a host cell, a recombinant protein expression vector, a protein production medium, a sample tube, a microplate, an instruction for users of the kit, and a transfection reagent.

<<Pharmaceutical Composition>>

A cysteine knot protein produced by the production method of the present invention can be used as a raw material of a pharmaceutical composition containing the cysteine knot protein as an active ingredient. The invention of the present application includes a method for producing the pharmaceutical composition, the method including bringing the cysteine knot protein and an excipient into contact. For the excipient, any component commonly known as an excipient to be contained in pharmaceutical compositions can be appropriately selected without limitation.

### Examples

Hereinafter, examples according to the present invention will be described, but the present invention is not limited thereto.

<<Preparation of Mammalian Expression Plasmid (Expression-Enhancing Vector) for Expression-Enhancing Factors>>

The following 15 genes were used as expression-enhancing factors (chaperone proteins) for examination.
(1) Human heat shock protein 90a (HSP90α) gene (HSP90AA1) (GenBank No. NP_001017963, amino acid sequence: SEQ ID NO: 2) (nucleotide sequence after codon optimization: SEQ ID NO: 1),
(2) Human HSP90α gene (HSP90AA1) (GenBank No. NP_005339, amino acid sequence: SEQ ID NO: 4) (nucleotide sequence after codon optimization: SEQ ID NO: 3),
(3) Chinese hamster HSP90α gene (GenBank No. NP_001233750, amino acid sequence: SEQ ID NO: 6) (nucleotide sequence after codon optimization: SEQ ID NO: 5),
(4) Human HSP9013 gene (HSP90AB1) (GenBank No. NP_001258899, amino acid sequence: SEQ ID NO: 8) (nucleotide sequence after codon optimization: SEQ ID NO: 7),
(5) Human HSP9013 gene (HSP90AB1) (GenBank No. NP_001258900, amino acid sequence: SEQ ID NO: 10) (nucleotide sequence after codon optimization: SEQ ID NO: 9),
(6) Human HSP9013 gene (HSP90AB1) (GenBank No. NP_001258901, amino acid sequence: SEQ ID NO: 12) (nucleotide sequence after codon optimization: SEQ ID NO: 11),
(7) Chinese hamster (CH) HSP9013 gene (GenBank No. XP 003501716, amino acid sequence: SEQ ID NO: 14) (nucleotide sequence after codon optimization: SEQ ID NO: 13),
(8) Human Cell Division Cycle 37, HSP90 cochaperone (CDC37) gene (Genbank No. NP_008996, amino acid sequence: SEQ ID NO: 16) (nucleotide sequence after codon optimization: SEQ ID NO: 15),
(9) Chinese hamster (CH) CDC37 gene (GenBank No. XP 003499785, amino acid sequence: SEQ ID NO: 18) (nucleotide sequence after codon optimization: SEQ ID NO: 17),
(10) Human HSP60 gene (GenBank No. NP_955472, amino acid sequence: SEQ ID NO: 20) (nucleotide sequence after codon optimization: SEQ ID NO: 19),
(11) Human HSP10 gene (GenBank No. NP_002148, amino acid sequence: SEQ ID NO: 24) (nucleotide sequence after codon optimization: SEQ ID NO: 23),
(12) Human HSP110 gene (GenBank No. NP_006635, amino acid sequence: SEQ ID NO: 26) (nucleotide sequence after codon optimization: SEQ ID NO: 25),
(13) HSP70 gene of Chinese hamster ovary-derived cell, CHO (J. Biotechnology 143 (2009) 34-43) (nucleotide sequence after codon optimization: SEQ ID NO: 27, amino acid sequence: SEQ ID NO: 28),
(14) HSP27 gene of Chinese hamster ovary-derived cell, CHO (J. Biotechnology 143 (2009) 34-43) (nucleotide sequence after codon optimization: SEQ ID NO: 29, amino acid sequence: SEQ ID NO: 30), and
(15) Human HSP40 gene (GenBank No. NP_001530, amino acid sequence: SEQ ID NO: 22) (nucleotide sequence after codon optimization: SEQ ID NO: 21)

For each of the 15 genes, an optimum nucleotide sequence for an expression system with CHO cells was determined by using OptimumGene (codon optimization) from GenScript. For each of the 15 genes, a Kozak sequence (ccacc) and a stop codon (TGA) were respectively added to the N terminus and C terminus of the determined optimum nucleotide sequence to produce a gene fragment through chemical synthesis. Each gene fragment was inserted into a HindIII-EcoRI site of a pcDNA3.1(+) vector (Cat. No. V79020, Invitrogen), which is an expression vector for mammals, to produce a plasmid vector for the expression-enhancing factor (1 mg/mL). Through the described steps, 15 expression-enhancing vectors were obtained.

As a control to the expression-enhancing factors, an Enhanced Green Fluorescent Protein (EGFP) gene (GenBank No. AAF62891.1) was used. For the EGFP gene, an optimum nucleotide sequence for an expression system with CHO cells was determined by using OptimumGene (codon optimization) from GenScript. A Kozak sequence (ccacc) and a stop codon (TGA) were respectively added to the N terminus and C terminus of the determined optimum nucleotide sequence to produce a gene fragment through chemical synthesis. The gene fragment was inserted into a HindIII-EcoRI site of a pcDNA3.1(+) vector (Cat. No. V79020, Invitrogen), which is an expression vector for mammals, to produce a plasmid vector for the control (1 mg/mL).

<<Preparation of Mammalian Expression Plasmids (Recombinant Protein Expression Vectors) for Cysteine Knot Proteins and Helix Bundle Cytokine>>

The following eight genes were used as genes belonging to the cysteine knot protein family for examination. The proteins encoded by the eight genes are each a protein having a cysteine knot motif
(1) Human Nerve Growth Factor (NGF) gene (GenBank No. NP_002497, amino acid sequence: SEQ ID NO: 32) (nucleotide sequence after codon optimization: SEQ ID NO: 31),
(2) Human Platelet-Derived Growth Factor 13 (PDGF-β) gene (GenBank No. NP_002599, amino acid sequence: SEQ ID NO: 34) (nucleotide sequence after codon optimization: SEQ ID NO: 33),
(3) Human Interleukin 17F (IL-17F) gene (GenBank No. NP_443104, amino acid sequence: SEQ ID NO: 36) (nucleotide sequence after codon optimization: SEQ ID NO: 35),
(4) Human Glial cell line-Derived Neurotrophic Factor (GDNF) gene (GenBank No. NP_000505, amino acid sequence: SEQ ID NO: 38) (nucleotide sequence after codon optimization: SEQ ID NO: 37),
(5) Human Neurotrophin 3 (NT3) gene (GenBank No. NP_002518, amino acid sequence: SEQ ID NO: 40) (nucleotide sequence after codon optimization: SEQ ID NO: 39),
(6) Human Brain-Derived Neurotrophic Factor (BDNF) gene (GenBank No. NP_733931, amino acid sequence: SEQ ID NO: 44) (nucleotide sequence after codon optimization: SEQ ID NO: 43),
(7) Gene encoding fusion protein of human BDNF and Fc fragment of human IgG1 heavy chain (hBDNF-Fc fusion protein) (nucleotide sequence after codon optimization: SEQ ID NO: 85, amino acid sequence: SEQ ID NO: 86), and
(8) Gene encoding fusion protein of human GDNF and Fc fragment of human IgG1 heavy chain (hGDNF-Fc fusion protein) (nucleotide sequence after codon optimization: SEQ ID NO: 99, amino acid sequence: SEQ ID NO: 100)

As a gene for a helix bundle cytokine, a human Interferon-7 (IFN-7) gene (GenBank No. NP_000610, amino acid sequence: SEQ ID NO: 42; Genbank No. NM_000619, wild-type nucleotide sequence: SEQ ID NO: 81) (nucleotide sequence after codon optimization: SEQ ID NO: 41) was used. Here, the human IFN-γ is not a cysteine knot protein, and hence corresponds to a comparative example.

For each of the nine genes, an optimum nucleotide sequence for an expression system with CHO cells was determined by using OptimumGene (codon optimization) from GenScript. For each of the nine genes, a Kozak sequence (ccacc) and a stop codon (TGA) were respectively added to the N terminus and C terminus of the determined optimum nucleotide sequence to produce a gene fragment through chemical synthesis. Each gene fragment was inserted into a HindIII-EcoRI site of a pcDNA3.1(+) vector (Cat. No. V79020, Invitrogen), which is an expression vector for mammals, to produce a plasmid vector for the recombinant protein (1 mg/mL). Through the described steps, nine recombinant protein expression vectors were obtained.

<<Examination of Enhancing Effects of Expression-Enhancing Factors in Production of Cysteine Knot Protein Family (Nerve Growth Factor; NGF) by Using Expi-CHO Expression System>>

The following operations were performed by using a Gibco™ Expi™ Expression System (Cat. No. A29133, ThermoFisher Scientific K.K.) in accordance with a Max Titer protocol. First, cultured Expi-CHO cells (6×10⁶ cells/mL) were added to a 125-mL Erlenmeyer flask (Corning Inc. Cat. No. 431143) containing ExpiCHO™ Expression Medium (Cat. No. A29100-01, ThermoFisher Scientific K.K.) (25 mL). Next, a reagent (1 ml) containing plasmid vectors shown in Table 1-1 below was prepared. Separately from the tube for the reagent containing plasmid vectors, Expifectamine (Cat. No. A12129) (80 μL) and OptiPRO™ SFM (Cat. No. 12309050) (920 μL) were added to another tube. The reagent containing plasmid vectors and the reagent containing Expifectamine were each stirred, and left to stand at room temperature for 5 minutes. Thereafter, the two reagents were slowly mixed together to form ExpiFectamine™ CHO/plasmid DNA complexes, which were left to stand at room temperature for 1 to 5 minutes. The complexes were added to the 125-mL Erlenmeyer flask containing Expi-CHO cells (6×10⁶ cells/mL), and stirring culture was performed at 37° C., 8% CO₂, and 125 rpm overnight.

TABLE 1-1

| | | | | Total |
|---|---|---|---|---|
| Sample | Cysteine knot protein | Chaperone protein or control | SFM* | volume |
| No. | (plasmid name) | (plasmid name) | (μL) | (μL) |
| 1-1 | pcDNA3.1(+)hNGF (1 mg/mL) 16 μL | pcDNA3.1(+)EGFP (1 mg/mL) 4 μL | 980 | 1000 |
| 1-2 | pcDNA3.1(+)hNGF (1 mg/mL) 16 μL | pcDNA3.1(+)hHSP90AA1_NP_001017963 (1 mg/mL) 4 μL | 980 | 1000 |
| 1-3 | pcDNA3.1(+)hNGF (1 mg/mL) 16 μL | pcDNA3.1(+)hHSP90AA1_NP_005339 (1 mg/mL) 4 μL | 980 | 1000 |
| 1-4 | pcDNA3.1(+)hNGF (1 mg/mL) 16 μL | pcDNA3.1(+)hHSP90AB1_NP_001258899 (1 mg/mL) 4 μL | 980 | 1000 |
| 1-5 | pcDNA3.1(+)hNGF (1 mg/mL) 16 μL | pcDNA3.1(+)hHSP90AB1_NP_001258900 (1 mg/mL) 4 μL | 980 | 1000 |
| 1-6 | pcDNA3.1(+)hNGF (1 mg/mL) 16 μL | pcDNA3.1(+)hHSP90AB1_NP_001258901 (1 mg/mL) 4 μL | 980 | 1000 |
| 1-7 | pcDNA3.1(+)hNGF (1 mg/mL) 16 μL | pcDNA3.1(+)hCDC37 (1 mg/mL) 4 μL | 980 | 1000 |
| 1-8 | pcDNA3.1(+)hNGF (1 mg/mL) 16 μL | pcDNA3.1(+)hHSP90AA1_NP_001017963 (1 mg/mL) 2 μL | 980 | 1000 |
| | | pcDNA3.1(+)hCDC37 (1 mg/mL) 2 μL | | |
| 1-9 | pcDNA3.1(+)hNGF (1 mg/mL) 16 μL | pcDNA3.1(+)CH-HSP90AA1 (1 mg/mL) 4 μL | 980 | 1000 |
| 1-10 | pcDNA3.1(+)hNGF (1 mg/mL) 16 μL | pcDNA3.1(+)CH-HSP90AB1 (1 mg/mL) 4 μL | 980 | 1000 |
| 1-11 | pcDNA3.1(+)hNGF (1 mg/mL) 16 μL | pcDNA3.1(+)CH-CDC37 (1 mg/mL) 4 μL | 980 | 1000 |
| 1-12 | pcDNA3.1(+)hNGF (1 mg/mL) 16 μL | pcDNA3.1(+)hHSP10 (1 mg/mL) 4 μL | 980 | 1000 |
| 1-13 | pcDNA3.1(+)hNGF (1 mg/mL) 16 μL | pcDNA3.1(+)hHSP60 (1 mg/mL) 4 μL | 980 | 1000 |
| 1-14 | pcDNA3.1(+)hNGF (1 mg/mL) 16 μL | pcDNA3.1(+)hHSP110 (1 mg/mL) 4 μL | 980 | 1000 |
| 1-15 | pcDNA3.1(+)hNGF (1 mg/mL) 16 μL | pcDNA3.1(+)CH-HSP70 (1 mg/mL) 4 μL | 980 | 1000 |
| 1-16 | pcDNA3.1(+)hNGF (1 mg/mL) 16 μL | pcDNA3.1(+)CH-HSP27(1 mg/mL) 4 μL | 980 | 1000 |

*OptiPRO (TM) SFM (Cat. No. 12309050)

Within 18 to 22 hours after transfecting with the plasmid vectors, ExpiFectamine™ CHO Enhancer (150 μL) and ExpiCHO™ Feed (4 mL) were added to each of the culture solutions, and culture was performed at 32° C., 5% CO₂, and 125 rpm. On day 5 of culture, ExpiCHO™ Feed (4 mL) was further added to each of the culture solutions, and culture was performed at 32° C., 5% CO₂, and 125 rpm. Between day 7 and day 13 of culture, the culture supernatants were collected, and the productivities for the cysteine knot protein were calculated through ELISA. The cell survival rates were calculated through measurement of total cell counts and viable cell counts by using a Countess II FL automatic cell counter (Cat. No. AMQAF1000, ThermoFisher Scientific K.K.).

The human NGF concentrations of the culture supernatants were calculated by using ELISA (Biosensis Pty Ltd., Cat. No. BEK-2212-1P/2P). On day 11 of culture, cells were collected, and the viable cell counts were determined by using a Countess II FL automatic cell counter, centrifugation was performed at 10,000×g for 5 minutes, and the culture supernatants were then collected. The culture supernatants collected were diluted to a degree that allowed quantification with standards (3.9 to 250 pg/mL) (dilution rate: 100,000-fold to 1,000,000-fold). To wells of a microplate, standards, a QC sample included in the kit, and the diluted culture supernatants each in 100 μL were added, and stirring was performed at room temperature for 45 minutes. Thereafter, the samples were removed from the wells, and the wells were washed five times with a washing solution (200 μL/well×5 times). A detection antibody was added at 100 μL/well, and stirring was performed at room temperature for 30 minutes. The antibody was removed from the wells, and washing was performed in the same manner. Tetramethylbenzidine reagent (TMB reagent) was added to the wells for reaction, and, after finding the occurrence of reaction with moderate blue coloring, a stop solution was added to the wells. The absorbances at a wavelength of 450 nm were measured by using a microplate reader (SpectraMax M5e, Molecular Devices LLC.), and the production levels (concentrations) of NGF in the culture supernatants were calculated from the standards to determine production-enhancing effects depending on the presence or absence of expression-enhancing factors (Table 1-2). The results revealed that co-expression of HSP90AA1 or HSP90AB1 and NGF caused NGF production-enhancing effect (sample Nos. 1-2 to 1-6, and 1-8 to 1-10). Likewise, NGF production-enhancing effect was found for Chinese hamster-derived CDC37 (sample No. 1-11). While gene transfer with CHO-derived HSP27 resulted in high ratios of viable cell count, expression of hCDC37 was found to lead to reduced viable cell counts (sample Nos. 1-7 and 1-16). For the other cases of gene transfer, no change was found in total cell count and viable cell count. Gene transfer with CDC37, HSP90AA1, or HSP90AB1 resulted in elevated NGF production levels per cell (sample Nos. 1-2 to 1-11).

TABLE 1-2

| | | NGF | Total | Viable | | Production |
|---|---|---|---|---|---|---|
| | | production | cell | cell | Viable | level |
| Sample | | level | count | count | cell | per cell |
| No. | Gene used for transfection (+NGF) | (μg/mL) | (cells/mL) | (cells/mL) | rate | (pg/cell) |
| 1-1 | EGFP (control) | 2.42 | 2.6 × 10⁷ | 1.1 × 10⁷ | 43% | 0.22 |
| 1-2 | hHSP90AA1_NP_001017963 | 2.91 | 2.6 × 10⁷ | 9.8 × 10⁶ | 38% | 0.30 |
| 1-3 | hHSP90AA1_NP_005339 | 3.35 | 1.7 × 10⁷ | 6.4 × 10⁶ | 37% | 0.52 |
| 1-4 | hHSP90AB1_NP_001258899 | 2.66 | 2.4 × 10⁷ | 1.0 × 10⁷ | 42% | 0.27 |
| 1-5 | hHSP90AB1_NP_001258900 | 2.55 | 1.8 × 10⁷ | 7.5 × 10⁶ | 41% | 0.34 |
| 1-6 | hHSP90AB1_NP_001258901 | 3.91 | 2.0 × 10⁷ | 8.5 × 10⁶ | 42% | 0.46 |
| 1-7 | hCDC37 | 2.30 | 3.2 × 10⁷ | 2.0 × 10⁶ | 6% | 1.14 |
| 1-8 | hHSP90AA1_NP_001017963/hCDC37 | 2.88 | 2.0 × 10⁷ | 7.3 × 10⁶ | 36% | 0.39 |
| 1-9 | CH-HSP90AA1 | 2.62 | 1.8 × 10⁷ | 8.1 × 10⁶ | 45% | 0.32 |
| 1-10 | CH-HSP90AB1 | 2.56 | 2.7 × 10⁷ | 1.1 × 10⁷ | 43% | 0.22 |
| 1-11 | CH-CDC37 | 2.92 | 2.2 × 10⁷ | 9.2 × 10⁶ | 42% | 0.32 |
| 1-12 | hHSP10 | 1.97 | 2.7 × 10⁷ | 7.4 × 10⁶ | 27% | 0.27 |
| 1-13 | hHSP60 | 2.63 | 2.7 × 10⁷ | 9.5 × 10⁶ | 35% | 0.28 |
| 1-14 | hHSP110 | 2.52 | 2.2 × 10⁷ | 9.5 × 10⁶ | 44% | 0.27 |
| 1-15 | CH-HSP70 | 1.12 | 2.8 × 10⁷ | 4.7 × 10⁶ | 17% | 0.24 |
| 1-16 | CH-HSP27 | 3.29 | 4.6 × 10⁷ | 2.9 × 10⁷ | 61% | 0.12 |

<<Examination of Enhancing Effects of Expression-Enhancing Factors in Production of Cysteine Knot Protein Family (Neurotrophin-3; NT3) by Using Expi-CHO Expression System>>

The following operations were performed by using a Gibco™ Expi™ Expression System (Cat. No. A29133, ThermoFisher Scientific K.K.) in accordance with a Max Titer protocol. First, cultured Expi-CHO cells (6×10⁶ cells/mL) were added to a 125-mL Erlenmeyer flask (Corning Inc. Cat. No. 431143) containing ExpiCHO™ Expression Medium (Cat. No. A29100-01, ThermoFisher Scientific K.K.) (25 mL). Next, a reagent (1 ml) containing plasmid vectors shown in Table 2-1 below was prepared. Separately from the tube for the reagent containing plasmid vectors, Expifectamine (Cat. No. A12129) (80 μL) and OptiPRO™ SFM (Cat. No. 12309050) (920 μL) were added to another tube. The reagent containing plasmid vectors and the reagent containing Expifectamine were each stirred, and left to stand at room temperature for 5 minutes. Thereafter, the two reagents were slowly mixed together to form ExpiFectamine™ CHO/plasmid DNA complexes, which were left to stand at room temperature for 1 to 5 minutes. The complexes were added to the 125-mL Erlenmeyer flask containing Expi-CHO cells, and stirring culture was performed at 37° C., 8% CO₂, and 125 rpm overnight.

TABLE 2-1

| | | | | Total |
|---|---|---|---|---|
| Sample | Cysteine knot protein | Chaperone protein or control | SFM* | volume |
| No. | (plasmid name) | (plasmid name) | (μL) | (μL) |
| 2-1 | pcDNA3.1(+)hNT3(1 mg/mL) 16 μL | pcDNA3.1(+)EGFP (1 mg/mL) 4 μL | 980 | 1000 |
| 2-2 | pcDNA3.1(+)hNT3(1 mg/mL) 16 μL | pcDNA3.1(+)hHSP90AA1_NP_001017963 (1 mg/mL) 4 μL | 980 | 1000 |
| 2-3 | pcDNA3.1(+)hNT3(1 mg/mL) 16 μL | pcDNA3.1(+)hHSP90AA1_NP_005339 (1 mg/mL) 4 μL | 980 | 1000 |
| 2-4 | pcDNA3.1(+)hNT3(1 mg/mL) 16 μL | pcDNA3.1(+)hHSP90AB1_NP_001258899 (1 mg/mL) 4 μL | 980 | 1000 |
| 2-5 | pcDNA3.1(+)hNT3(1 mg/mL) 16 μL | pcDNA3.1(+)hHSP90AB1_NP_001258900 (1 mg/mL) 4 μL | 980 | 1000 |
| 2-6 | pcDNA3.1(+)hNT3(1 mg/mL) 16 μL | pcDNA3.1(+)hHSP90AB1_NP_001258901 (1 mg/mL) 4 μL | 980 | 1000 |
| 2-7 | pcDNA3.1(+)hNT3(1 mg/mL) 16 μL | pcDNA3.1(+)hCDC37 (1 mg/mL) 4 μL | 980 | 1000 |
| 2-8 | pcDNA3.1(+)hNT3(1 mg/mL) 16 μL | pcDNA3.1(+)hHSP90AA1_NP_001017963 (1 mg/mL) 2 μL | 980 | 1000 |
| | | pcDNA3.1(+)hCDC37 (1 mg/mL) 2 μL | | |
| 2-9 | pcDNA3.1(+)hNT3(1 mg/mL) 16 μL | pcDNA3.1(+)CH-HSP90AA1 (1 mg/mL) 4 μL | 980 | 1000 |
| 2-10 | pcDNA3.1(+)hNT3(1 mg/mL) 16 μL | pcDNA3.1(+)CH-HSP90AB1 (1 mg/mL) 4 μL | 980 | 1000 |
| 2-11 | pcDNA3.1(+)hNT3(1 mg/mL) 16 μL | pcDNA3.1(+)CH-CDC37 (1 mg/mL) 4 μL | 980 | 1000 |
| 2-12 | pcDNA3.1(+)hNT3(1 mg/mL) 16 μL | pcDNA3.1(+)hHSP10 (1 mg/mL) 4 μL | 980 | 1000 |
| 2-13 | pcDNA3.1(+)hNT3(1 mg/mL) 16 μL | pcDNA3.1(+)hHSP60 (1 mg/mL) 4 μL | 980 | 1000 |
| 2-14 | pcDNA3.1(+)hNT3(1 mg/mL) 16 μL | pcDNA3.1(+)hHSP110 (1 mg/mL) 4 μL | 980 | 1000 |
| 2-15 | pcDNA3.1(+)hNT3(1 mg/mL) 16 μL | pcDNA3.1(+)CH-HSP70 (1 mg/mL) 4 μL | 980 | 1000 |
| 2-16 | pcDNA3.1(+)hNT3(1 mg/mL) 16 μL | pcDNA3.1(+)CH-HSP27(1 mg/mL) 4 μL | 980 | 1000 |

*OptiPRO (TM) SFM (Cat. No. 12309050)

Within 18 to 22 hours after transfecting with the plasmid vectors, ExpiFectamine™ CHO Enhancer (150 μL) and ExpiCHO™ Feed (4 mL) were added to each of the culture solutions, and culture was performed at 32° C., 5% CO₂, and 125 rpm. On day 5 of culture, ExpiCHO™ Feed (4 mL) was further added to each of the culture solutions, and culture was performed at 32° C., 5% CO₂, and 125 rpm. Between day 7 and day 13 of culture, the culture supernatants were collected, and the productivities for the cysteine knot protein were calculated through ELISA. The cell survival rates were calculated through measurement of total cell counts and viable cell counts by using a Countess II FL automatic cell counter (Cat. No. AMQAF1000, ThermoFisher Scientific K.K.).

The human NT-3 concentrations of the culture supernatants were calculated by using ELISA (Biosensis Pty Ltd., Cat. No. BEK-2221-1P/2P). On day 12 of culture, cells were collected, and the viable cell counts were determined by using a Countess II FL automatic cell counter, centrifugation was performed at 10,000×g for 5 minutes, and the culture supernatants were then collected. The culture supernatants collected were diluted to a degree that allowed quantification with standards (15.6 to 1000 pg/mL) (dilution rate: 10,000-fold to 100,000-fold). To wells of a microplate, standards, a QC sample included in the kit, and the diluted culture supernatants each in 100 μL were added, and stirring was performed at room temperature for 45 minutes. Thereafter, the samples were removed from the wells, and the wells were washed five times with a washing solution (200 μL/well×5 times). A detection antibody was added at 100 μL/well, and stirring was performed at room temperature for 30 minutes. The antibody was removed from the wells, and washing was performed in the same manner. TMB reagent was added to the wells for reaction, and, after finding the occurrence of reaction with moderate blue coloring, a stop solution was added to the wells. The absorbances at a wavelength of 450 nm were measured by using a microplate reader (SpectraMax M5e, Molecular Devices LLC.), and the production levels (concentrations) of NT3 in the culture supernatants were calculated from the standards to determine production-enhancing effects depending on the presence or absence of expression-enhancing factors (Table 2-2). The results found NT3 production-enhancing effect for all the factors used for gene transfer. In particular, the samples subjected to gene transfer with CHO-HSP70, CHO-HSP27, HSP90AA1, or HSP90AB1 exhibited high productivity for NT3 per cell (sample Nos. 2-2, 2-6, 2-15, and 2-16).

TABLE 2-2

| | | NT3 | Total | Viable | | Production |
|---|---|---|---|---|---|---|
| | | production | cell | cell | Viable | level |
| Sample | | level | count | count | cell | per cell |
| No. | Gene used for transfection (+NT3) | (μg/mL) | (cells/mL) | (cells/mL) | rate | (pg/cell) |
| 2-1 | EGFP (control) | 1.0 | 2.0 × 10⁷ | 6.0 × 10⁶ | 30% | 0.2 |
| 2-2 | hHSP90AA1_NP_001017963 | 16.6 | 2.0 × 10⁷ | 6.0 × 10⁶ | 34% | 2.7 |
| 2-3 | hHSP90AA1_NP_005339 | 10.1 | 2.0 × 10⁷ | 8.0 × 10⁶ | 36% | 1.3 |
| 2-4 | hHSP90AB1_NP_001258899 | 11.5 | 2.0 × 10⁷ | 7.0 × 10⁶ | 30% | 1.7 |
| 2-5 | hHSP90AB1_NP_001258900 | 11.3 | 2.0 × 10⁷ | 6.0 × 10⁶ | 35% | 1.8 |
| 2-6 | hHSP90AB1_NP_001258901 | 17.5 | 2.0 × 10⁷ | 6.0 × 10⁶ | 29% | 3.1 |
| 2-7 | hCDC37 | 11.2 | 2.0 × 10⁷ | 8.0 × 10⁶ | 48% | 1.5 |
| 2-8 | hHSP90AA1_NP_001017963/hCDC37 | 11.1 | 2.0 × 10⁷ | 7.0 × 10⁶ | 44% | 1.7 |
| 2-9 | CH-HSP90AA1 | 12.2 | 2.0 × 10⁷ | 7.0 × 10⁶ | 35% | 1.7 |
| 2-10 | CH-HSP90AB1 | 10.9 | 2.0 × 10⁷ | 6.0 × 10⁶ | 32% | 1.8 |
| 2-11 | CH-CDC37 | 9.8 | 2.0 × 10⁷ | 7.0 × 10⁶ | 38% | 1.5 |
| 2-12 | hHSP10 | 9.2 | 4.0 × 10⁷ | 8.0 × 10⁶ | 23% | 1.1 |
| 2-13 | hHSP60 | 9.1 | 2.0 × 10⁷ | 6.0 × 10⁶ | 35% | 1.4 |
| 2-14 | hHSP110 | 6.7 | 2.0 × 10⁷ | 7.0 × 10⁶ | 41% | 0.9 |
| 2-15 | CH-HSP70 | 17.3 | 2.0 × 10⁷ | 7.0 × 10⁶ | 29% | 2.5 |
| 2-16 | CH-HSP27 | 17.6 | 2.0 × 10⁷ | 6.0 × 10⁶ | 28% | 3.1 |

<<Examination of Enhancing Effects of Expression-Enhancing Factors in Production of Cysteine Knot Protein Family (Interleukin 17F; IL-17F) by Using Expi-CHO Expression System>>

The following operations were performed by using a Gibco™ Expi™ Expression System (Cat. No. A29133, ThermoFisher Scientific K.K.) in accordance with a Max Titer protocol. First, cultured Expi-CHO cells (6×10⁶ cells/mL) were added to a 125-mL Erlenmeyer flask (Corning Inc. Cat. No. 431143) containing ExpiCHO™ Expression Medium (Cat. No. A29100-01, ThermoFisher Scientific K.K.) (25 mL). Next, a reagent (1 ml) containing plasmid vectors shown in Table 3-1 below was prepared. Separately from the tube for the reagent containing plasmid vectors, Expifectamine (Cat. No. A12129) (80 μL) and OptiPRO™ SFM (Cat. No. 12309050) (920 μL) were added to another tube. The reagent containing plasmid vectors and the reagent containing Expifectamine were each stirred, and left to stand at room temperature for 5 minutes. Thereafter, the two reagents were slowly mixed together to form ExpiFectamine™ CHO/plasmid DNA complexes, which were left to stand at room temperature for 1 to 5 minutes. The complexes were added to the 125-mL Erlenmeyer flask containing Expi-CHO cells, and stirring culture was performed at 37° C., 8% CO₂, and 125 rpm overnight.

TABLE 3-1

| | | | | Total |
|---|---|---|---|---|
| Sample | Cysteine knot protein | Chaperone protein or control | SFM* | volume |
| No. | (plasmid name) | (plasmid name) | (μL) | (μL) |
| 3-1 | pcDNA3.1(+)hIL17F(1 mg/mL) 16 μL | pcDNA3.1(+)EGFP (1 mg/mL) 4 μL | 980 | 1000 |
| 3-2 | pcDNA3.1(+)hIL17F(1 mg/mL) 16 μL | pcDNA3.1(+)hHSP90AA1_NP_001017963 (1 mg/mL) 4 μL | 980 | 1000 |
| 3-3 | pcDNA3.1(+)hIL17F(1 mg/mL) 16 μL | pcDNA3.1(+)hHSP90AA1_NP_005339 (1 mg/mL) 4 μL | 980 | 1000 |
| 3-4 | pcDNA3.1(+)hIL17F(1 mg/mL) 16 μL | pcDNA3.1(+)hHSP90AB1_NP_001258899 (1 mg/mL) 4 μL | 980 | 1000 |
| 3-5 | pcDNA3.1(+)hIL17F(1 mg/mL) 16 μL | pcDNA3.1(+)hHSP90AB1_NP_001258900 (1 mg/mL) 4 μL | 980 | 1000 |
| 3-6 | pcDNA3.1(+)hIL17F(1 mg/mL) 16 μL | pcDNA3.1(+)hHSP90AB1_NP_001258901 (1 mg/mL) 4 μL | 980 | 1000 |
| 3-7 | pcDNA3.1(+)hIL17F(1 mg/mL) 16 μL | pcDNA3.1(+)hCDC37 (1 mg/mL) 4 μL | 980 | 1000 |
| 3-8 | pcDNA3.1(+)hIL17F(1 mg/mL) 16 μL | pcDNA3.1(+)hHSP90AA1_NP_001017963 (1 mg/mL) 2 μL | 980 | 1000 |
| | | pcDNA3.1(+)hCDC37 (1 mg/mL) 2 μL | | |
| 3-9 | pcDNA3.1(+)hIL17F(1 mg/mL) 16 μL | pcDNA3.1(+)CH-HSP90AA1 (1 mg/mL) 4 μL | 980 | 1000 |
| 3-10 | pcDNA3.1(+)hIL17F(1 mg/mL) 16 μL | pcDNA3.1(+)CH-HSP90AB1 (1 mg/mL) 4 μL | 980 | 1000 |
| 3-11 | pcDNA3.1(+)hIL17F(1 mg/mL) 16 μL | pcDNA3.1(+)CH-CDC37 (1 mg/mL) 4 μL | 980 | 1000 |
| 3-12 | pcDNA3.1(+)hIL17F(1 mg/mL) 16 μL | pcDNA3.1(+)hHSP10 (1 mg/mL) 4 μL | 980 | 1000 |
| 3-13 | pcDNA3.1(+)hIL17F(1 mg/mL) 16 μL | pcDNA3.1(+)hHSP60 (1 mg/mL) 4 μL | 980 | 1000 |
| 3-14 | pcDNA3.1(+)hIL17F(1 mg/mL) 16 μL | pcDNA3.1(+)hHSP110 (1 mg/mL) 4 μL | 980 | 1000 |
| 3-15 | pcDNA3.1(+)hIL17F(1 mg/mL) 16 μL | pcDNA3.1(+)CH-HSP70 (1 mg/mL) 4 μL | 980 | 1000 |
| 3-16 | pcDNA3.1(+)hIL17F(1 mg/mL) 16 μL | pcDNA3.1(+)CH-HSP27(1 mg/mL) 4 μL | 980 | 1000 |

*OptiPRO (TM) SFM (Cat. No. 12309050)

Within 18 to 22 hours after transfecting with the plasmid vectors, ExpiFectamine™ CHO Enhancer (150 μL) and ExpiCHO™ Feed (4 mL) were added to each of the culture solutions, and culture was performed at 32° C., 5% CO₂, and 125 rpm. On day 5 of culture, ExpiCHO™ Feed (4 mL) was further added to each of the culture solutions, and culture was performed at 32° C., 5% CO₂, and 125 rpm. Between day 7 and day 13 of culture, the culture supernatants were collected, and the productivities for the cysteine knot protein were calculated through ELISA. The cell survival rates were calculated through measurement of total cell counts and viable cell counts by using a Countess II FL automatic cell counter (Cat. No. AMQAF1000, ThermoFisher Scientific K.K.).

The human IL-17F concentrations of the culture supernatants were calculated by using ELISA (Invitrogen, Cat. No. BMS2037-2). On day 12 of culture, cells were collected, and the viable cell counts were determined by using a Countess II FL automatic cell counter, centrifugation was performed at 10,000×g for 5 minutes, and the culture supernatants were then collected. The culture supernatants collected were diluted to a degree that allowed quantification with standards (15.6 to 1000 pg/mL) (dilution rate: 10,000-fold to 100,000-fold). To wells of a microplate, standards and the diluted culture supernatants each in 50 μL were added, and stirring was performed at room temperature for 2 hours. Thereafter, the samples were removed from the wells, and the wells were washed four times with a washing solution (300 μL/well×4 times). A biotinylated antibody was added at 50 μL/well, and stirring was performed at room temperature for 2 hours. The antibody was removed from the wells, and washing was performed in the same manner. Streptavidin-HRP was added at 50 μL/well, and stirring was performed at room temperature for 2 hours. Streptavidin-HRP was removed from the wells, washing was performed in the same manner, TMB reagent was added to the wells for reaction, and, after finding the occurrence of reaction with moderate blue coloring, a stop solution was added to the wells. The absorbances at a wavelength of 450 nm were measured by using a microplate reader (SpectraMax M5e, Molecular Devices LLC.), and the production levels (concentrations) of IL-17F in the culture supernatants were calculated from the standards to determine production-enhancing effects depending on the presence or absence of expression-enhancing factors (Table 3-2). The results found IL-17F production-enhancing effect for all the factors. The samples subjected to gene transfer with CHO-HSP27, HSP90AA1, HSP90AB1, HSP90AA1 and CDC37, HSP10, CDC37, CH-HSP90AB1, CH-CDC37, HSP60, or HSP110 exhibited particularly high IL-17F productivity per cell (sample Nos. 3-3, 3-5, 3-6, 3-7, 3-8, 3-10, 3-11, 3-12, 3-13, 3-14, and 3-16).

TABLE 3-2

| | | hIL17F | Total | Viable | | Production |
|---|---|---|---|---|---|---|
| | | production | cell | cell | Viable | level |
| Sample | | level | count | count | cell | per cell |
| No. | Gene used for transfection (+hIL17F) | (μg/mL) | (cells/mL) | (cells/mL) | rate | (pg/cell) |
| 3-1 | EGFP (control) | 8.5 | 4.0 × 10⁷ | 2.0 × 10⁶ | 6% | 3.6 |
| 3-2 | hHSP90AA1_NP_001017963 | 11.5 | 3.0 × 10⁷ | 2.0 × 10⁶ | 5% | 6.3 |
| 3-3 | hHSP90AA1_NP_005339 | 15.8 | 3.0 × 10⁷ | 1.0 × 10⁶ | 4% | 11.3 |
| 3-4 | hHSP90AB1_NP_001258899 | 14.6 | 3.0 × 10⁷ | 2.0 × 10⁶ | 7% | 6.6 |
| 3-5 | hHSP90AB1_NP_001258900 | 11.6 | 3.0 × 10⁷ | 2.0 × 10⁶ | 6% | 6.5 |
| 3-6 | hHSP90AB1_NP_001258901 | 14.7 | 3.0 × 10⁷ | 7.0 × 10⁵ | 2% | 21.8 |
| 3-7 | hCDC37 | 10.7 | 3.0 × 10⁷ | 6.0 × 10⁵ | 2% | 17.0 |
| 3-8 | hHSP90AA1_NP_001017963/hCDC37 | 17.4 | 4.0 × 10⁷ | 9.0 × 10⁵ | 3% | 19.5 |
| 3-9 | CH-HSP90AA1 | 11.4 | 3.0 × 10⁷ | 1.0 × 10⁶ | 3% | 10.7 |
| 3-10 | CH-HSP90AB1 | 14.5 | 3.0 × 10⁷ | 8.0 × 10⁵ | 3% | 17.3 |
| 3-11 | CH-CDC37 | 14.3 | 3.0 × 10⁷ | 9.0 × 10⁵ | 3% | 15.5 |
| 3-12 | hHSP10 | 17.6 | 3.0 × 10⁷ | 8.0 × 10⁵ | 3% | 22.3 |
| 3-13 | hHSP60 | 21.0 | 3.0 × 10⁷ | 8.0 × 10⁵ | 3% | 25.2 |
| 3-14 | hHSP110 | 16.6 | 3.0 × 10⁷ | 1.0 × 10⁶ | 5% | 11.7 |
| 3-15 | CH-HSP70 | 10.9 | 5.0 × 10⁷ | 3.0 × 10⁶ | 5% | 4.1 |
| 3-16 | CH-HSP27 | 13.8 | 4.0 × 10⁷ | 1.0 × 10⁶ | 3% | 12.9 |

<<Examination of Enhancing Effects of Expression-Enhancing Factors in Production of Cysteine Knot Protein Family (Platelet-Derived Growth Factor-13; PDGF-β) by Using Expi-CHO Expression System>>

The following operations were performed by using a Gibco™ Expi™ Expression System (Cat. No. A29133, ThermoFisher Scientific K.K.) in accordance with a Max Titer protocol. First, cultured Expi-CHO cells (6×10⁶ cells/mL) were added to a 125-mL Erlenmeyer flask (Corning Inc. Cat. No. 431143) containing ExpiCHO™ Expression Medium (Cat. No. A29100-01, ThermoFisher Scientific K.K.) (25 mL). Next, a reagent (1 ml) containing plasmid vectors shown in Table 4-1 below was prepared. Separately from the tube for the reagent containing plasmid vectors, Expifectamine (Cat. No. A12129) (80 μL) and OptiPRO™ SFM (Cat. No. 12309050) (920 μL) were added to another tube. The reagent containing plasmid vectors and the reagent containing Expifectamine were each stirred, and left to stand at room temperature for 5 minutes. Thereafter, the two reagents were slowly mixed together to form ExpiFectamine™ CHO/plasmid DNA complexes, which were left to stand at room temperature for 1 to 5 minutes. The complexes were added to the 125-mL Erlenmeyer flask containing Expi-CHO cells, and stirring culture was performed at 37° C., 8% CO₂, and 125 rpm overnight.

TABLE 4-1

| | | | | Total |
|---|---|---|---|---|
| Sample | Cysteine knot protein | Chaperone protein or control | SFM* | volume |
| No. | (plasmid name) | (plasmid name) | (μL) | (μL) |
| 4-1 | pcDNA3.1(+)hPDGFβ (1 mg/mL) 16 μL | pcDNA3.1(+)EGFP (1 mg/mL) 4 μL | 980 | 1000 |
| 4-2 | pcDNA3.1(+)hPDGFβ (1 mg/mL) 16 μL | pcDNA3.1(+)hHSP90AA1_NP_001017963 (1 mg/mL) 4 μL | 980 | 1000 |
| 4-3 | pcDNA3.1(+)hPDGFβ (1 mg/mL) 16 μL | pcDNA3.1(+)hHSP90AA1_NP_005339 (1 mg/mL) 4 μL | 980 | 1000 |
| 4-4 | pcDNA3.1(+)hPDGFβ (1 mg/mL) 16 μL | pcDNA3.1(+)hHSP90AB1_NP_001258899 (1 mg/mL) 4 μL | 980 | 1000 |
| 4-5 | pcDNA3.1(+)hPDGFβ (1 mg/mL) 16 μL | pcDNA3.1(+)hHSP90AB1_NP_001258900 (1 mg/mL) 4 μL | 980 | 1000 |
| 4-6 | pcDNA3.1(+)hPDGFβ (1 mg/mL) 16 μL | pcDNA3.1(+)hHSP90AB1_NP_001258901 (1 mg/mL) 4 μL | 980 | 1000 |
| 4-7 | pcDNA3.1(+)hPDGFβ (1 mg/mL) 16 μL | pcDNA3.1(+)hCDC37 (1 mg/mL) 4 μL | 980 | 1000 |
| 4-8 | pcDNA3.1(+)hPDGFβ (1 mg/mL) 16 μL | pcDNA3.1(+)hHSP90AA1_NP_001017963 (1 mg/mL) 2 μL | 980 | 1000 |
| | | pcDNA3.1(+)hCDC37 (1 mg/mL) 2 μL | | |
| 4-9 | pcDNA3.1(+)hPDGFβ (1 mg/mL) 16 μL | pcDNA3.1(+)CH-HSP90AA1 (1 mg/mL) 4 μL | 980 | 1000 |
| 4-10 | pcDNA3.1(+)hPDGFβ (1 mg/mL) 16 μL | pcDNA3.1(+)CH-HSP90AB1 (1 mg/mL) 4 μL | 980 | 1000 |
| 4-11 | pcDNA3.1(+)hPDGFβ (1 mg/mL) 16 μL | pcDNA3.1(+)CH-CDC37 (1 mg/mL) 4 μL | 980 | 1000 |
| 4-12 | pcDNA3.1(+)hPDGFβ (1 mg/mL) 16 μL | pcDNA3.1(+)hHSP10 (1 mg/mL) 4 μL | 980 | 1000 |
| 4-13 | pcDNA3.1(+)hPDGFβ (1 mg/mL) 16 μL | pcDNA3.1(+)hHSP60 (1 mg/mL) 4 μL | 980 | 1000 |
| 4-14 | pcDNA3.1(+)hPDGFβ (1 mg/mL) 16 μL | pcDNA3.1(+)hHSP110 (1 mg/mL) 4 μL | 980 | 1000 |
| 4-15 | pcDNA3.1(+)hPDGFβ (1 mg/mL) 16 μL | pcDNA3.1(+)CH-HSP70 (1 mg/mL) 4 μL | 980 | 1000 |
| 4-16 | pcDNA3.1(+)hPDGFβ (1 mg/mL) 16 μL | pcDNA3.1(+)CH-HSP27(1 mg/mL) 4 μL | 980 | 1000 |

*OptiPRO (TM) SFM (Cat. No. 12309050)

Within 18 to 22 hours after transfecting with the plasmid vectors, ExpiFectamine™ CHO Enhancer (150 μL) and ExpiCHO™ Feed (4 mL) were added to each of the culture solutions, and culture was performed at 32° C., 5% CO₂, and 125 rpm. On day 5 of culture, ExpiCHO™ Feed (4 mL) was further added to each of the culture solutions, and culture was performed at 32° C., 5% CO₂, and 125 rpm. Between day 7 and day 13 of culture, the culture supernatants were collected, and the productivities for the cysteine knot protein were calculated through ELISA. The cell survival rates were calculated through measurement of total cell counts and viable cell counts by using a Countess II FL automatic cell counter (Cat. No. AMQAF1000, ThermoFisher Scientific K.K.).

The human PDGF-β concentrations of the culture supernatants were calculated by using ELISA (Novus Biologicals, LLC, Cat. No. KA1760). On day 7 of culture, cells were collected, and the viable cell counts were determined by using a Countess II FL automatic cell counter, centrifugation was performed at 10,000×g for 5 minutes, and the culture supernatants were then collected. The culture supernatants collected were diluted to a degree that allowed quantification with standards (0.549 to 400 pg/mL) (dilution rate: 10,000-fold to 100,000-fold). To wells of a microplate, standards and the diluted culture supernatants each in 100 μL were added, and stirring was performed at 4° C. overnight. Thereafter, the samples were removed from the wells, and the wells were washed four times with a washing solution (300 μL/well×4 times). A biotinylated antibody was added at 100 μL/well, and stirring was performed at room temperature for 60 minutes. The antibody was removed from the well, washing was performed in the same manner, streptavidin-HRP was added at 100 μL/well, and stirring was performed at room temperature for 45 minutes. Streptavidin-HRP was removed from the wells, washing was performed in the same manner, TMB reagent was added to the wells for reaction, and, after finding the occurrence of reaction with moderate blue coloring, a stop solution was added to the wells. The absorbances at a wavelength of 450 nm were measured by using a microplate reader (SpectraMax M5e, Molecular Devices LLC.), and the production levels (concentrations) of PDGF-β in the culture supernatants were calculated from the standards to determine production-enhancing effects depending on the presence or absence of expression-enhancing factors (Table 4-2). The results revealed that co-expression with CH-HSP70, CH-CDC37, or CH-HSP27 caused PDGF-β production-enhancing effect (sample Nos. 4-11, 4-15, 4-16). Regarding PDGF-β production levels per cell, enhanced production was found for the samples subjected to gene transfer with CH-HSP27 or hHSP90AA1 (sample Nos. 4-2, 4-16).

TABLE 4-2

| | | hPDGFβ | Total | Viable | | Production |
|---|---|---|---|---|---|---|
| | | production | cell | cell | Viable | level |
| Sample | | level | count | count | cell | per cell |
| No. | Gene used for transfection (+hPDGFβ) | (μg/mL) | (cells/mL) | (cells/mL) | rate | (pg/cell) |
| 4-1 | EGFP (control) | 3.4 | 3.0 × 10⁷ | 1.0 × 10⁷ | 37% | 0.3 |
| 4-2 | hHSP90AA1_NP_001017963 | 3.1 | 5.0 × 10⁷ | 5.0 × 10⁶ | 11% | 0.6 |
| 4-3 | hHSP90AA1_NP_005339 | 2.3 | 3.0 × 10⁷ | 1.0 × 10⁷ | 38% | 0.2 |
| 4-4 | hHSP90AB1_NP_001258899 | 2.1 | 2.0 × 10⁷ | 9.0 × 10⁶ | 37% | 0.2 |
| 4-5 | hHSP90AB1_NP_001258900 | 2.1 | 4.0 × 10⁷ | 9.0 × 10⁶ | 23% | 0.2 |
| 4-6 | hHSP90AB1_NP_001258901 | 2.6 | 2.0 × 10⁷ | 9.0 × 10⁶ | 38% | 0.3 |
| 4-7 | hCDC37 | 2.6 | 3.0 × 10⁷ | 1.0 × 10⁷ | 40% | 0.2 |
| 4-8 | hHSP90AA1_NP_001017963/hCDC37 | 2.6 | 3.0 × 10⁷ | 1.0 × 10⁷ | 34% | 0.3 |
| 4-9 | CH-HSP90AA1 | 3.0 | 3.0 × 10⁷ | 9.0 × 10⁶ | 28% | 0.3 |
| 4-10 | CH-HSP90AB1 | 3.1 | 3.0 × 10⁷ | 1.0 × 10⁷ | 38% | 0.3 |
| 4-11 | CH-CDC37 | 3.5 | 2.0 × 10⁷ | 8.0 × 10⁶ | 49% | 0.4 |
| 4-12 | hHSP10 | 3.0 | 3.0 × 10⁷ | 8.0 × 10⁶ | 24% | 0.4 |
| 4-13 | hHSP60 | 2.7 | 2.0 × 10⁷ | 8.0 × 10⁶ | 40% | 0.3 |
| 4-14 | hHSP110 | 2.5 | 3.0 × 10⁷ | 7.0 × 10⁶ | 25% | 0.3 |
| 4-15 | CH-HSP70 | 3.9 | 3.0 × 10⁷ | 1.0 × 10⁷ | 39% | 0.3 |
| 4-16 | CH-HSP27 | 3.5 | 2.0 × 10⁷ | 7.0 × 10⁶ | 39% | 0.5 |

<<Examination of Enhancing Effects of Expression-Enhancing Factors in Production of Cysteine Knot Protein Family (Glial Cell Line-Derived Neurotrophic Factor; GDNF) by Using Expi-CHO Expression System>>

The following operations were performed by using a Gibco™ Expi™ Expression System (Cat. No. A29133, ThermoFisher Scientific K.K.) in accordance with a Max Titer protocol. First, cultured Expi-CHO cells (6×10⁶ cells/mL) were added to a 125-mL Erlenmeyer flask (Corning Inc. Cat. No. 431143) containing ExpiCHO™ Expression Medium (Cat. No. A29100-01, ThermoFisher Scientific K.K.) (25 mL). Next, a reagent (1 ml) containing plasmid vectors shown in Table 5-1 below was prepared. Separately from the tube for the reagent containing plasmid vectors, Expifectamine (Cat. No. A12129) (80 μL) and OptiPRO™ SFM (Cat. No. 12309050) (920 μL) were added to another tube. The reagent containing plasmid vectors and the reagent containing Expifectamine were each stirred, and left to stand at room temperature for 5 minutes. Thereafter, the two reagents were slowly mixed together to form ExpiFectamine™ CHO/plasmid DNA complexes, which were left to stand at room temperature for 1 to 5 minutes. The complexes were added to the 125-mL Erlenmeyer flask containing Expi-CHO cells, and stirring culture was performed at 37° C., 8% CO₂, and 125 rpm overnight.

TABLE 5-1

| | | | | Total |
|---|---|---|---|---|
| Sample | Cysteine knot protein | Chaperone protein or control | SFM* | volume |
| No. | (plasmid name) | (plasmid name) | (μL) | (μL) |
| 5-1 | pcDNA3.1(+)hGDNF (1 mg/mL) 16 μL | pcDNA3.1(+)EGFP (1 mg/mL) 4 μL | 980 | 1000 |
| 5-2 | pcDNA3.1(+)hGDNF (1 mg/mL) 16 μL | pcDNA3.1(+)hHSP90AA1_NP_001017963 (1 mg/mL) 4 μL | 980 | 1000 |
| 5-3 | pcDNA3.1(+)hGDNF (1 mg/mL) 16 μL | pcDNA3.1(+)hHSP90AA1_NP_005339 (1 mg/mL) 4 μL | 980 | 1000 |
| 5-4 | pcDNA3.1(+)hGDNF (1 mg/mL) 16 μL | pcDNA3.1(+)hHSP90AB1_NP_001258899 (1 mg/mL) 4 μL | 980 | 1000 |
| 5-5 | pcDNA3.1(+)hGDNF (1 mg/mL) 16 μL | pcDNA3.1(+)hHSP90AB1_NP_001258900 (1 mg/mL) 4 μL | 980 | 1000 |
| 5-6 | pcDNA3.1(+)hGDNF (1 mg/mL) 16 μL | pcDNA3.1(+)hHSP90AB1_NP_001258901 (1 mg/mL) 4 μL | 980 | 1000 |
| 5-7 | pcDNA3.1(+)hGDNF (1 mg/mL) 16 μL | pcDNA3.1(+)hCDC37 (1 mg/mL) 4 μL | 980 | 1000 |
| 5-8 | pcDNA3.1(+)hGDNF (1 mg/mL) 16 μL | pcDNA3.1(+)hHSP90AA1_NP_001017963 (1 mg/mL) 2 μL | 980 | 1000 |
| | | pcDNA3.1(+)hCDC37 (1 mg/mL) 2 μL | | |
| 5-9 | pcDNA3.1(+)hGDNF (1 mg/mL) 16 μL | pcDNA3.1(+)CH-HSP90AA1 (1 mg/mL) 4 μL | 980 | 1000 |
| 5-10 | pcDNA3.1(+)hGDNF (1 mg/mL) 16 μL | pcDNA3.1(+)CH-HSP90AB1 (1 mg/mL) 4 μL | 980 | 1000 |
| 5-11 | pcDNA3.1(+)hGDNF (1 mg/mL) 16 μL | pcDNA3.1(+)CH-CDC37 (1 mg/mL) 4 μL | 980 | 1000 |
| 5-12 | pcDNA3.1(+)hGDNF (1 mg/mL) 16 μL | pcDNA3.1(+)hHSP10 (1 mg/mL) 4 μL | 980 | 1000 |
| 5-13 | pcDNA3.1(+)hGDNF (1 mg/mL) 16 μL | pcDNA3.1(+)hHSP60 (1 mg/mL) 4 μL | 980 | 1000 |
| 5-14 | pcDNA3.1(+)hGDNF (1 mg/mL) 16 μL | pcDNA3.1(+)hHSP110 (1 mg/mL) 4 μL | 980 | 1000 |
| 5-15 | pcDNA3.1(+)hGDNF (1 mg/mL) 16 μL | pcDNA3.1(+)CH-HSP70 (1 mg/mL) 4 μL | 980 | 1000 |
| 5-16 | pcDNA3.1(+)hGDNF (1 mg/mL) 16 μL | pcDNA3.1(+)CH-HSP27(1 mg/mL) 4 μL | 980 | 1000 |
| 5-17 | pcDNA3.1(+)hGDNF (1 mg/mL) 16 μL | pcDNA3.1(+)hHSP40 (1 mg/mL) 4 μL | 980 | 1000 |

*OptiPRO (TM) SFM (Cat. No. 12309050)

Within 18 to 22 hours after transfecting with the plasmid vectors, ExpiFectamine™ CHO Enhancer (150 μL) and ExpiCHO™ Feed (4 mL) were added to each of the culture solutions, and culture was performed at 32° C., 5% CO₂, and 125 rpm. On day 5 of culture, ExpiCHO™ Feed (4 mL) was further added to each of the culture solutions, and culture was performed at 32° C., 5% CO₂, and 125 rpm. Between day 7 and day 13 of culture, the culture supernatants were collected, and the productivities for the cysteine knot protein were calculated through ELISA. The cell survival rates were calculated through measurement of total cell counts and viable cell counts by using a Countess II FL automatic cell counter (Cat. No. AMQAF1000, ThermoFisher Scientific K.K.).

The human GDNF concentrations of the culture supernatants were calculated by using ELISA (Biosensis Pty Ltd., Cat. No. BEK-2222-1P/2P). On day 7 and day 12 of culture, cells were collected. On day 12, the viable cell counts were determined by using a Countess II FL automatic cell counter. The culture supernatants collected were centrifuged at 10,000×g for 5 minutes, and then collected. The culture supernatants collected were diluted to a degree that allowed quantification with standards (7.8 to 500 pg/mL) (dilution rate: 100,000-fold to 1,000,000-fold). To wells of a microplate, standards, a QC sample included in the kit, and the diluted culture supernatants each in 100 μL were added, and stirring was performed at room temperature for 45 minutes. Thereafter, the samples were removed from the wells, and the wells were washed five times with a washing solution (200 μL/well×5 times). A detection antibody was added at 100 μL/well, and stirring was performed at room temperature for 30 minutes. The antibody was removed from the wells, and washing was performed in the same manner. TMB reagent was added to the wells for reaction, and, after finding the occurrence of reaction with moderate blue coloring, a stop solution was added to the wells. The absorbances at a wavelength of 450 nm were measured by using a microplate reader (SpectraMax M5e, Molecular Devices LLC.), and the production levels (concentrations) of GDNF in the culture supernatants were the presence or absence of expression-enhancing factors (Table 5-2). The results showed that gene transfer with CH-HSP70, CH-HSP27, HSP90AB1, HSP10, or CH-HSP90AA resulted in increased GDNF production levels per cell on day 12 of culture (sample Nos. 5-5, 5-9, 5-12, 5-15, and 5-16).

The results in Table 5-2 were considered to show incorrect GDNF production levels because the dilution rates were improper in the protein quantification by ELISA, and hence the same experiment was again conducted to determine GDNF production-enhancing effects depending on the presence or absence of expression-enhancing factors (Table 5-3). The results found that all the chaperone proteins used gave GDNF production-enhancing effect and increased GDNF production levels per cell (Table 5-3, sample Nos. 5-1 to 5-17).

The recombinant proteins used in the five experiments described above are proteins belonging to different families in terms of the classification in FIG. 2, but are proteins belonging in common to the cysteine knot protein super family. Accordingly, the results of the five experiments described above suggested that in allowing the mammalian cells to produce a cysteine knot protein, co-expression with any of the above specific chaperone proteins can give enhanced production levels for the cysteine knot protein.

TABLE 5-2

| | | hGDNF | Total | Viable | | Production |
|---|---|---|---|---|---|---|
| | | production | cell | cell | Viable | level |
| Sample | | level | count | count | cell | per cell |
| No. | Gene used for transfection (+hGDNF) | (μg/mL) | (cells/mL) | (cells/mL) | rate | (pg/cell) |
| 5-1 | EGFP (control) | 82.6 | 2.0 × 10⁷ | 7.0 × 10⁶ | 30% | 11.4 |
| 5-2 | hHSP90AA1_NP_001017963 | 68.5 | 2.0 × 10⁷ | 7.0 × 10⁶ | 30% | 9.7 |
| 5-3 | hHSP90AA1_NP_005339 | 69.0 | 2.0 × 10⁷ | 8.0 × 10⁶ | 34% | 8.8 |
| 5-4 | hHSP90AB1_NP_001258899 | 60.3 | 3.0 × 10⁷ | 8.0 × 10⁶ | 29% | 7.4 |
| 5-5 | hHSP90AB1_NP_001258900 | 61.3 | 3.0 × 10⁷ | 4.0 × 10⁶ | 16% | 14.4 |
| 5-6 | hHSP90AB1_NP_001258901 | 73.0 | 2.0 × 10⁷ | 7.0 × 10⁶ | 29% | 11.2 |
| 5-7 | hCDC37 | 78.7 | 3.0 × 10⁷ | 8.0 × 10⁶ | 29% | 9.5 |
| 5-8 | hHSP90AA1_NP_001017963/hCDC37 | 75.6 | 2.0 × 10⁷ | 9.0 × 10⁶ | 41% | 8.1 |
| 5-9 | CH-HSP90AA1 | 69.4 | 3.0 × 10⁷ | 6.0 × 10⁶ | 17% | 12.5 |
| 5-10 | CH-HSP90AB1 | 61.6 | 3.0 × 10⁷ | 6.0 × 10⁶ | 23% | 9.7 |
| 5-11 | CH-CDC37 | 61.3 | 2.0 × 10⁷ | 6.0 × 10⁶ | 30% | 10.7 |
| 5-12 | hHSP10 | 54.4 | 2.0 × 10⁷ | 4.0 × 10⁶ | 19% | 14.9 |
| 5-13 | hHSP60 | 44.4 | 3.0 × 10⁷ | 5.0 × 10⁶ | 16% | 9.2 |
| 5-14 | hHSP110 | 45.7 | 3.0 × 10⁷ | 6.0 × 10⁶ | 22% | 7.8 |
| 5-15 | CH-HSP70 | 81.3 | 1.0 × 10⁷ | 5.0 × 10⁶ | 38% | 17.7 |
| 5-16 | CH-HSP27 | 71.8 | 4.0 × 10⁷ | 3.0 × 10⁶ | 6% | 27.1 |

TABLE 5-3

| | | hGDNF | Total | Viable | | Production |
|---|---|---|---|---|---|---|
| | | production | cell | cell | Viable | level |
| Sample | | level | count | count | cell | per cell |
| No. | Gene used for transfection (+hGDNF) | (μg/mL) | (cells/mL) | (cells/mL) | rate | (pg/cell) |
| 5-1 | EGFP (control) | 3.2 | 1.4 × 10⁷ | 4.0 × 10⁶ | 28.3% | 0.81 |
| 5-2 | hHSP90AA1_NP_001017963 | 26.9 | 1.4 × 10⁷ | 7.3 × 10⁶ | 53.3% | 3.67 |
| 5-3 | hHSP90AA1_NP_005339 | 23.7 | 1.3 × 10⁷ | 7.2 × 10⁶ | 54.6% | 3.29 |
| 5-4 | hHSP90AB1_NP_001258899 | 24.7 | 1.6 × 10⁷ | 5.8 × 10⁶ | 37.6% | 4.23 |
| 5-5 | hHSP90AB1_NP_001258900 | 21.0 | 1.2 × 10⁷ | 8.1 × 10⁶ | 67.2% | 2.59 |
| 5-6 | hHSP90AB1_NP_001258901 | 14.3 | 9.4 × 10⁶ | 5.3 × 10⁶ | 56.2% | 2.71 |
| 5-7 | hCDC37 | 21.1 | 1.7 × 10⁷ | 6.7 × 10⁶ | 39.3% | 3.16 |
| 5-8 | hHSP90AA1_NP_001017963/hCDC37 | 17.2 | 1.3 × 10⁷ | 6.3 × 10⁶ | 48.6% | 2.74 |
| 5-9 | CH-HSP90AA1 | 19.6 | 1.0 × 10⁷ | 5.4 × 10⁶ | 53.3% | 3.63 |
| 5-10 | CH-HSP90AB1 | 18.1 | 1.6 × 10⁷ | 5.3 × 10⁶ | 33.4% | 3.4 |
| 5-11 | CH-CDC37 | 19.6 | 2.1 × 10⁷ | 5.0 × 10⁶ | 24.2% | 3.92 |
| 5-12 | hHSP10 | 20.1 | 1.7 × 10⁷ | 3.7 × 10⁶ | 21.5% | 5.41 |
| 5-13 | hHSP60 | 21.0 | 1.7 × 10⁷ | 4.0 × 10⁶ | 23.5% | 5.27 |
| 5-14 | hHSP110 | 19.6 | 1.8 × 10⁷ | 4.4 × 10⁶ | 24.9% | 4.5 |
| 5-15 | CH-HSP70 | 19.0 | 2.2 × 10⁷ | 4.5 × 10⁶ | 20.2% | 4.25 |
| 5-16 | CH-HSP27 | 24.6 | 1.6 × 10⁷ | 8.0 × 10⁶ | 50.2% | 3.08 |
| 5-17 | hHSP40 | 18.7 | 1.3 × 10⁷ | 3.5 × 10⁶ | 27.2% | 5.32 |

<<Examination of Enhancing Effects of Expression-Enhancing Factors in Production of Helix Bundle Cytokine (Interferon-γ; IFN-γ) by Using Expi-CHO Expression System>>

The following operations were performed by using a Gibco™ Expi™ Expression System (Cat. No. A29133, ThermoFisher Scientific K.K.) in accordance with a Max Titer protocol. First, cultured Expi-CHO cells (6×10⁶ cells/mL) were added to a 125-mL Erlenmeyer flask (Coming Inc. Cat. No. 431143) containing ExpiCHO™ Expression Medium (Cat. No. A29100-01, ThermoFisher Scientific K.K.) (25 mL). Next, a reagent (1 ml) containing plasmid vectors shown in Table 6-1 below was prepared. Separately from the tube for the reagent containing plasmid vectors, Expifectamine (Cat. No. A12129) (80 μL) and OptiPRO™ SFM (Cat. No. 12309050) (920 μL) were added to another tube. The reagent containing plasmid vectors and the reagent containing Expifectamine were each stirred, and left to stand at room temperature for 5 minutes. Thereafter, the two reagents were slowly mixed together to form ExpiFectamine™ CHO/plasmid DNA complexes, which were left to stand at room temperature for 1 to 5 minutes. The complexes were added to the 125-mL Erlenmeyer flask containing Expi-CHO cells, and stirring culture was performed at 37° C., 8% CO₂, and 125 rpm overnight.

TABLE 6-1

| | | | | Total |
|---|---|---|---|---|
| Sample | Helix bundle cytokine | Chaperone protein or control | SFM* | volume |
| No. | (plasmid name) | (plasmid name) | (μL) | (μL) |
| 6-1 | pcDNA3.1(+)hIFNγ (1 mg/mL) 16 μL | pcDNA3.1(+)EGFP (1 mg/mL) 4 μL | 980 | 1000 |
| 6-2 | pcDNA3.1(+)hIFNγ (1 mg/mL) 16 μL | pcDNA3.1(+)hHSP90AA1_NP_001017963 (1 mg/mL) 4 μL | 980 | 1000 |
| 6-3 | pcDNA3.1(+)hIFNγ (1 mg/mL) 16 μL | pcDNA3.1(+)hHSP90AA1_NP_005339 (1 mg/mL) 4 μL | 980 | 1000 |
| 6-4 | pcDNA3.1(+)hIFNγ (1 mg/mL) 16 μL | pcDNA3.1(+)hHSP90AB1_NP_001258899 (1 mg/mL) 4 μL | 980 | 1000 |
| 6-5 | pcDNA3.1(+)hIFNγ (1 mg/mL) 16 μL | pcDNA3.1(+)hHSP90AB1_NP_001258900 (1 mg/mL) 4 μL | 980 | 1000 |
| 6-6 | pcDNA3.1(+)hIFNγ (1 mg/mL) 16 μL | pcDNA3.1(+)hHSP90AB1_NP_001258901 (1 mg/mL) 4 μL | 980 | 1000 |
| 6-7 | pcDNA3.1(+)hIFNγ (1 mg/mL) 16 μL | pcDNA3.1(+)hCDC37 (1 mg/mL) 4 μL | 980 | 1000 |
| 6-8 | pcDNA3.1(+)hIFNγ (1 mg/mL) 16 μL | pcDNA3.1(+)hHSP90AA1_NP_001017963 (1 mg/mL) 2 μL | 980 | 1000 |
| | | pcDNA3.1(+)hCDC37 (1 mg/mL) 2 μL | | |
| 6-9 | pcDNA3.1(+)hIFNγ (1 mg/mL) 16 μL | pcDNA3.1(+)CH-HSP90AA1 (1 mg/mL) 4 μL | 980 | 1000 |
| 6-10 | pcDNA3.1(+)hIFNγ (1 mg/mL) 16 μL | pcDNA3.1(+)CH-HSP90AB1 (1 mg/mL) 4 μL | 980 | 1000 |
| 6-11 | pcDNA3.1(+)hIFNγ (1 mg/mL) 16 μL | pcDNA3.1(+)CH-CDC37 (1 mg/mL) 4 μL | 980 | 1000 |
| 6-12 | pcDNA3.1(+)hIFNγ (1 mg/mL) 16 μL | pcDNA3.1(+)hHSP10 (1 mg/mL) 4 μL | 980 | 1000 |
| 6-13 | pcDNA3.1(+)hIFNγ (1 mg/mL) 16 μL | pcDNA3.1(+)hHSP60 (1 mg/mL) 4 μL | 980 | 1000 |
| 6-14 | pcDNA3.1(+)hIFNγ (1 mg/mL) 16 μL | pcDNA3.1(+)hHSP110 (1 mg/mL) 4 μL | 980 | 1000 |
| 6-15 | pcDNA3.1(+)hIFNγ (1 mg/mL) 16 μL | pcDNA3.1(+)CH-HSP70 (1 mg/mL) 4 μL | 980 | 1000 |
| 6-16 | pcDNA3.1(+)hIFNγ (1 mg/mL) 16 μL | pcDNA3.1(+)CH-HSP27(1 mg/mL) 4 μL | 980 | 1000 |

*OptiPRO (TM) SFM (Cat. No. 12309050)

Within 18 to 22 hours after transfecting with the plasmid vectors, ExpiFectamine™ CHO Enhancer (150 μL) and ExpiCHO™ Feed (4 mL) were added to each of the culture solutions, and culture was performed at 32° C., 5% CO₂, and 125 rpm. On day 5 of culture, ExpiCHO™ Feed (4 mL) was further added to each of the culture solutions, and culture was performed at 32° C., 5% CO₂, and 125 rpm. Between day 7 and day 13 of culture, the culture supernatants were collected, and the productivities for the helix bundle cytokine were calculated through ELISA. The cell survival rates were calculated through measurement of total cell counts and viable cell counts by using a Countess II FL automatic cell counter (Cat. No. AMQAF1000, ThermoFisher Scientific K.K.).

The human IFN-γ concentrations of the culture supernatants were calculated by using ELISA (Invitrogen, Cat. No. EHIFNG). On day 12 of culture, cells were collected, and the viable cell counts were determined by using a Countess II FL automatic cell counter, centrifugation was performed at 10,000×g for 5 minutes, and the culture supernatants were then collected. The culture supernatants collected were diluted to a degree that allowed quantification with standards (4.1 to 1000 pg/mL) (dilution rate: 10,000-fold to 100,000-fold). To wells of a microplate, standards and the diluted culture supernatants each in 50 μL were added, and stirring was performed at room temperature for 2 hours. Thereafter, the samples were removed from the wells, and the wells were washed four times with a washing solution (300 μL/well×4 times). A biotinylated antibody was added at 50 μL/well, and stirring was performed at room temperature for 2 hours. The antibody was removed from the wells, and washing was performed in the same manner. Streptavidin-HRP was added at 50 μL/well, and stirring was performed at room temperature for 2 hours. Streptavidin-HRP was removed from the wells, washing was performed in the same manner, TMB reagent was added to the wells for reaction, and, after finding the occurrence of reaction with moderate blue coloring, a stop solution was added to the wells. The absorbances at a wavelength of 450 nm were measured by using a microplate reader (SpectraMax M5e, Molecular Devices LLC.), and the production levels (concentrations) of IFN-γ in the culture supernatants were calculated from the standards to determine production-enhancing effects depending on the presence or absence of expression-enhancing factors (Table 6-2). The results found no IFN-γ production-enhancing effect for any of the genes. On the other hand, it was found that, as reported by Lee et al. (Journal of Biotechnology 143 (2009) 34-43 (NPL 1)), the gene expression of HSP70 resulted in an increased IFN-γ production level per cell (sample No. 6-15). Only one isoform of HSP90AB1 (HSP90(3) was found to exhibit activity, whereas the other molecules thereof were not (sample No. 6-6).

The results of the six experiments described above revealed that the chaperone proteins including HSP90 and CDC37 (examples) have enhancing effects for production of the cysteine knot proteins, but not for production of the helix bundle cytokine (comparative example).

TABLE 6-2

| | | hIFNγ | Total | Viable | | Production |
|---|---|---|---|---|---|---|
| | | production | cell | cell | Viable | level |
| Sample | | level | count | count | cell | per cell |
| No. | Gene used for transfection (+hIFNγ) | (μg/mL) | (cells/mL) | (cells/mL) | rate | (pg/cell) |
| 6-1 | EGFP (control) | 79.1 | 3.0 × 10⁷ | 6.0 × 10⁶ | 24% | 12.6 |
| 6-2 | hHSP90AA1_NP_001017963 | 55.1 | 3.0 × 10⁷ | 5.0 × 10⁶ | 20% | 10.1 |
| 6-3 | hHSP90AA1_NP_005339 | 66.0 | 3.0 × 10⁷ | 6.0 × 10⁶ | 17% | 11.5 |
| 6-4 | hHSP90AB1_NP_001258899 | 58.0 | 3.0 × 10⁷ | 8.0 × 10⁶ | 30% | 7.5 |
| 6-5 | hHSP90AB1_NP_001258900 | 39.8 | 2.0 × 10⁷ | 9.0 × 10⁶ | 39% | 4.7 |
| 6-6 | hHSP90AB1_NP_001258901 | 90.6 | 2.0 × 10⁷ | 7.0 × 10⁶ | 31% | 13.7 |
| 6-7 | hCDC37 | 65.7 | 5.0 × 10⁷ | 7.0 × 10⁶ | 14% | 10.0 |
| 6-8 | hHSP90AA1_NP_001017963/hCDC37 | 64.5 | 3.0 × 10⁷ | 7.0 × 10⁶ | 25% | 9.6 |
| 6-9 | CH-HSP90AA1 | 63.9 | 2.0 × 10⁷ | 6.0 × 10⁶ | 24% | 11.0 |
| 6-10 | CH-HSP90AB1 | 46.3 | 2.0 × 10⁷ | 6.0 × 10⁶ | 33% | 7.1 |
| 6-11 | CH-CDC37 | 61.9 | 2.0 × 10⁷ | 7.0 × 10⁶ | 35% | 8.6 |
| 6-12 | hHSP10 | 54.2 | 3.0 × 10⁷ | 7.0 × 10⁶ | 22% | 8.1 |
| 6-13 | hHSP60 | 40.1 | 2.0 × 10⁷ | 8.0 × 10⁶ | 38% | 4.7 |
| 6-14 | hHSP110 | 37.0 | 2.0 × 10⁷ | 6.0 × 10⁶ | 25% | 6.3 |
| 6-15 | CH-HSP70 | 73.4 | 1.0 × 10⁷ | 4.0 × 10⁶ | 24% | 20.7 |
| 6-16 | CH-HSP27 | 79.6 | 3.0 × 10⁷ | 6.0 × 10⁶ | 20% | 14.3 |

<<Examination of Enhancing Effects of Expression-Enhancing Factors in Production of Cysteine Knot Protein Family (Brain-Derived Neurotrophic Factor; BDNF) by Using Expi-CHO Expression System>>

The following operations were performed by using a Gibco™ Expi™ Expression System (Cat. No. A29133, ThermoFisher Scientific K.K.) in accordance with a Max Titer protocol. First, cultured Expi-CHO cells (6×10⁶ cells/mL) were added to a 125-mL Erlenmeyer flask (Corning Inc. Cat. No. 431143) containing ExpiCHO™ Expression Medium (Cat. No. A29100-01, ThermoFisher Scientific K.K.) (25 mL). Next, a reagent (1 ml) containing plasmid vectors shown in Table 7-1 below was prepared. Separately from the tube for the reagent containing plasmid vectors, Expifectamine (Cat. No. A12129) (80 μL) and OptiPRO™ SFM (Cat. No. 12309050) (920 μL) were added to another tube. The reagent containing plasmid vectors and the reagent containing Expifectamine were each stirred, and left to stand at room temperature for 5 minutes. Thereafter, the two reagents were slowly mixed together to form ExpiFectamine™ CHO/plasmid DNA complexes, which were left to stand at room temperature for 1 to 5 minutes. The complexes were added to the 125-mL Erlenmeyer flask containing Expi-CHO cells, and stirring culture was performed at 37° C., 8% CO₂, and 125 rpm overnight.

TABLE 7-1

| | | | | Total |
|---|---|---|---|---|
| Sample | Cysteine knot protein | Chaperone protein or control | SFM* | volume |
| No. | (plasmid name) | (plasmid name) | (μL) | (μL) |
| 7-1 | pcDNA3.1(+)hBDNF (1 mg/mL) 16 μL | pcDNA3.1(+)EGFP (1 mg/mL) 4 μL | 980 | 1000 |
| 7-2 | pcDNA3.1(+)hBDNF (1 mg/mL) 16 μL | pcDNA3.1(+)hHSP90AA1_NP_001017963 (1 mg/mL) 4 μL | 980 | 1000 |
| 7-3 | pcDNA3.1(+)hBDNF (1 mg/mL) 16 μL | pcDNA3.1(+)hHSP90AA1_NP_005339 (1 mg/mL) 4 μL | 980 | 1000 |
| 7-4 | pcDNA3.1(+)hBDNF (1 mg/mL) 16 μL | pcDNA3.1(+)hHSP90AB1_NP_001258899 (1 mg/mL) 4 μL | 980 | 1000 |
| 7-5 | pcDNA3.1(+)hBDNF (1 mg/mL) 16 μL | pcDNA3.1(+)hHSP90AB1_NP_001258900 (1 mg/mL) 4 μL | 980 | 1000 |
| 7-6 | pcDNA3.1(+)hBDNF (1 mg/mL) 16 μL | pcDNA3.1(+)hHSP90AB1_NP_001258901 (1 mg/mL) 4 μL | 980 | 1000 |
| 7-7 | pcDNA3.1(+)hBDNF (1 mg/mL) 16 μL | pcDNA3.1(+)hCDC37 (1 mg/mL) 4 μL | 980 | 1000 |
| 7-8 | pcDNA3.1(+)hBDNF (1 mg/mL) 16 μL | pcDNA3.1(+)hHSP90AA1_NP_001017963 (1 mg/mL) 2 μL | 980 | 1000 |
| | | pcDNA3.1(+)hCDC37 (1 mg/mL) 2 μL | | |
| 7-9 | pcDNA3.1(+)hBDNF (1 mg/mL) 16 μL | pcDNA3.1(+)CH-HSP90AA1 (1 mg/mL) 4 μL | 980 | 1000 |
| 7-10 | pcDNA3.1(+)hBDNF (1 mg/mL) 16 μL | pcDNA3.1(+)CH-HSP90AB1 (1 mg/mL) 4 μL | 980 | 1000 |
| 7-11 | pcDNA3.1(+)hBDNF (1 mg/mL) 16 μL | pcDNA3.1(+)CH-CDC37 (1 mg/mL) 4 μL | 980 | 1000 |
| 7-12 | pcDNA3.1(+)hBDNF (1 mg/mL) 16 μL | pcDNA3.1(+)hHSP10 (1 mg/mL) 4 μL | 980 | 1000 |
| 7-13 | pcDNA3.1(+)hBDNF (1 mg/mL) 16 μL | pcDNA3.1(+)hHSP60 (1 mg/mL) 4 μL | 980 | 1000 |
| 7-14 | pcDNA3.1(+)hBDNF (1 mg/mL) 16 μL | pcDNA3.1(+)hHSP110 (1 mg/mL) 4 μL | 980 | 1000 |
| 7-15 | pcDNA3.1(+)hBDNF (1 mg/mL) 16 μL | pcDNA3.1(+)CH-HSP70 (1 mg/mL) 4 μL | 980 | 1000 |
| 7-16 | pcDNA3.1(+)hBDNF (1 mg/mL) 16 μL | pcDNA3.1(+)CH-HSP27(1 mg/mL) 4 μL | 980 | 1000 |

*OptiPRO (TM) SFM (Cat. No. 12309050)

Within 18 to 22 hours after transfecting with the plasmid vectors, ExpiFectamine™ CHO Enhancer (150 μL) and ExpiCHO™ Feed (4 mL) were added to each of the culture solutions, and culture was performed at 32° C., 5% CO₂, and 125 rpm. On day 5 of culture, ExpiCHO™ Feed (4 mL) was further added to each of the culture solutions, and culture was performed at 32° C., 5% CO₂, and 125 rpm. Between day 7 and day 13 of culture, the culture supernatants were collected, and the productivities for the cysteine knot protein were calculated through ELISA. The cell survival rates were calculated through measurement of total cell counts and viable cell counts by using a Countess II FL automatic cell counter (Cat. No. AMQAF1000, ThermoFisher Scientific K.K.).

The human BDNF concentrations of the culture supernatants were calculated by using ELISA (Biosensis Pty Ltd., Cat. No. BEK-2211-1P/2P). On day 12 of culture, cells were collected, and the viable cell counts were determined by using a Countess II FL automatic cell counter, centrifugation was performed at 10,000×g for 5 minutes, and the culture supernatants were then collected. The culture supernatants collected were diluted to a degree that allowed quantification with standards (7.8 to 500 pg/mL) (dilution rate: 100,000-fold to 1,000,000-fold). To wells of a microplate, standards, a QC sample included in the kit, and the diluted culture supernatants each in 100 μL were added, and stirring was performed at room temperature for 45 minutes. Thereafter, the samples were removed from the wells, and the wells were washed five times with a washing solution (200 μL/well×5 times). A detection antibody was added at 100 μL/well, and stirring was performed at room temperature for 30 minutes. The antibody was removed from the wells, and washing was performed in the same manner. TMB reagent was added to the wells for reaction, and, after finding the occurrence of reaction with moderate blue coloring, a stop solution was added to the wells. The absorbances at a wavelength of 450 nm were measured by using a microplate reader (SpectraMax M5e, Molecular Devices LLC.), and the production levels (concentrations) of BDNF in the culture supernatants were calculated from the standards to determine production-enhancing effects depending on the presence or absence of expression-enhancing factors (Table 7-2). The results revealed BDNF production-enhancing effect caused by co-expression of HSP90AA1 or HSP90AB1 and BDNF (sample Nos. 7-2 to 7-6). BDNF production-enhancing effect was similarly found for Chinese hamster-derived HSP90AA1, HSP90AB1, and CDC37 (sample Nos. 7-9 to 7-11). Co-expression of HSP90AA1 and CDC37 gave the most increased production level (sample No. 7-8). BDNF production-enhancing effect was found also for HSP70, HSP27, HSP60, HSP10, and HSP110 (sample Nos. 7-12 to 7-16).

TABLE 7-2

| | | hBDNF | Total | Viable | | Production |
|---|---|---|---|---|---|---|
| | | production | cell | cell | Viable | level |
| Sample | | level | count | count | cell | per cell |
| No. | Gene used for transfection (+hBDNF) | (μg/mL) | (cells/mL) | (cells/mL) | rate | (pg/cell) |
| 7-1 | EGFP (control) | 0.87 | 1.4 × 10⁷ | 5.9 × 10⁶ | 42% | 0.15 |
| 7-2 | hHSP90AA1_NP_001017963 | 4.19 | 1.4 × 10⁷ | 6.7 × 10⁶ | 49% | 0.62 |
| 7-3 | hHSP90AA1_NP_005339 | 5.14 | 1.6 × 10⁷ | 5.8 × 10⁶ | 37% | 0.88 |
| 7-4 | hHSP90AB1_NP_001258899 | 5.31 | 1.2 × 10⁷ | 4.6 × 10⁶ | 38% | 1.15 |
| 7-5 | hHSP90AB1_NP_001258900 | 4.80 | 0.94 × 10⁷ | 3.9 × 10⁶ | 42% | 1.23 |
| 7-6 | hHSP90AB1_NP_001258901 | 5.57 | 1.0 × 10⁷ | 4.9 × 10⁶ | 48% | 1.14 |
| 7-7 | hCDC37 | 4.28 | 1.7 × 10⁷ | 6.5 × 10⁶ | 38% | 0.66 |
| 7-8 | hHSP90AA1_NP_001017963/hCDC37 | 6.42 | 1.3 × 10⁷ | 4.3 × 10⁶ | 32% | 1.50 |
| 7-9 | CH-HSP90AA1 | 6.23 | 1.6 × 10⁷ | 7.9 × 10⁶ | 50% | 0.79 |
| 7-10 | CH-HSP90AB1 | 3.26 | 2.1 × 10⁷ | 8.0 × 10⁶ | 39% | 0.41 |
| 7-11 | CH-CDC37 | 4.51 | 1.7 × 10⁷ | 7.2 × 10⁶ | 41% | 0.63 |
| 7-12 | hHSP10 | 4.35 | 1.7 × 10⁷ | 5.8 × 10⁶ | 34% | 0.76 |
| 7-13 | hHSP60 | 5.61 | 1.8 × 10⁷ | 7.3 × 10⁶ | 42% | 0.77 |
| 7-14 | hHSP110 | 4.07 | 1.3 × 10⁷ | 4.5 × 10⁶ | 35% | 0.91 |
| 7-15 | CH-HSP70 | 6.22 | 2.2 × 10⁷ | 7.1 × 10⁶ | 32% | 0.87 |
| 7-16 | CH-HSP27 | 5.47 | 1.6 × 10⁷ | 5.2 × 10⁶ | 33% | 1.06 |

<<Examination of Enhancing Effects of Expression-Enhancing Factors in Production of hBDNF-Fc Fusion Protein by Using Expi-CHO Expression System>>

The following operations were performed by using a Gibco™ Expi™ Expression System (Cat. No. A29133, ThermoFisher Scientific K.K.) in accordance with a Max Titer protocol. First, cultured Expi-CHO cells (6×10⁶ cells/mL) were added to a 125-mL Erlenmeyer flask (Corning Inc. Cat. No. 431143) containing ExpiCHO™ Expression Medium (Cat. No. A29100-01, ThermoFisher Scientific K.K.) (25 mL). Next, a reagent (1 ml) containing plasmid vectors shown in Table 8-1 below was prepared. Separately from the tube for the reagent containing plasmid vectors, Expifectamine (Cat. No. A12129) (80 μL) and OptiPRO™ SFM (Cat. No. 12309050) (920 μL) were added to another tube. The reagent containing plasmid vectors and the reagent containing Expifectamine were each stirred, and left to stand at room temperature for 5 minutes. Thereafter, the two reagents were slowly mixed together to form ExpiFectamine™ CHO/plasmid DNA complexes, which were left to stand at room temperature for 1 to 5 minutes. The complexes were added to the 125-mL Erlenmeyer flask containing Expi-CHO cells, and stirring culture was performed at 37° C., 8% CO₂, and 125 rpm overnight.

TABLE 8-1

| | | | | Total |
|---|---|---|---|---|
| Sample | Cysteine knot protein | Chaperone protein or control | SFM* | volume |
| No. | (plasmid name) | (plasmid name) | (μL) | (μL) |
| 8-1 | pcDNA3.1(+)hBDNF-Fc (1 mg/mL) 16 μL | pcDNA3.1(+)EGFP (1 mg/mL) 4 μL | 980 | 1000 |
| 8-2 | pcDNA3.1(+)hBDNF-Fc (1 mg/mL) 16 μL | pcDNA3.1(+)hHSP90AA1_NP_001017963 (1 mg/mL) 4 μL | 980 | 1000 |
| 8-3 | pcDNA3.1(+)hBDNF-Fc (1 mg/mL) 16 μL | pcDNA3.1(+)hHSP90AA1_NP_005339 (1 mg/mL) 4 μL | 980 | 1000 |
| 8-4 | pcDNA3.1(+)hBDNF-Fc (1 mg/mL) 16 μL | pcDNA3.1(+)hHSP90AB1_NP_001258899 (1 mg/mL) 4 μL | 980 | 1000 |
| 8-5 | pcDNA3.1(+)hBDNF-Fc (1 mg/mL) 16 μL | pcDNA3.1(+)hHSP90AB1_NP_001258900 (1 mg/mL) 4 μL | 980 | 1000 |
| 8-6 | pcDNA3.1(+)hBDNF-Fc (1 mg/mL) 16 μL | pcDNA3.1(+)hHSP90AB1_NP_001258901 (1 mg/mL) 4 μL | 980 | 1000 |
| 8-7 | pcDNA3.1(+)hBDNF-Fc (1 mg/mL) 16 μL | pcDNA3.1(+)hCDC37 (1 mg/mL) 4 μL | 980 | 1000 |
| 8-8 | pcDNA3.1(+)hBDNF-Fc (1 mg/mL) 16 μL | pcDNA3.1(+)hHSP90AA1_NP_001017963 (1 mg/mL) 2 μL | 980 | 1000 |
| | | pcDNA3.1(+)hCDC37 (1 mg/mL) 2 μL | | |
| 8-9 | pcDNA3.1(+)hBDNF-Fc (1 mg/mL) 16 μL | pcDNA3.1(+)CH-HSP90AA1 (1 mg/mL) 4 μL | 980 | 1000 |
| 8-10 | pcDNA3.1(+)hBDNF-Fc (1 mg/mL) 16 μL | pcDNA3.1(+)CH-HSP90AB1 (1 mg/mL) 4 μL | 980 | 1000 |
| 8-11 | pcDNA3.1(+)hBDNF-Fc (1 mg/mL) 16 μL | pcDNA3.1(+)CH-CDC37 (1 mg/mL) 4 μL | 980 | 1000 |
| 8-12 | pcDNA3.1(+)hBDNF-Fc (1 mg/mL) 16 μL | pcDNA3.1(+)hHSP10 (1 mg/mL) 4 μL | 980 | 1000 |
| 8-13 | pcDNA3.1(+)hBDNF-Fc (1 mg/mL) 16 μL | pcDNA3.1(+)hHSP60 (1 mg/mL) 4 μL | 980 | 1000 |
| 8-14 | pcDNA3.1(+)hBDNF-Fc (1 mg/mL) 16 μL | pcDNA3.1(+)hHSP110 (1 mg/mL) 4 μL | 980 | 1000 |
| 8-15 | pcDNA3.1(+)hBDNF-Fc (1 mg/mL) 16 μL | pcDNA3.1(+)CH-HSP70 (1 mg/mL) 4 μL | 980 | 1000 |
| 8-16 | pcDNA3.1(+)hBDNF-Fc (1 mg/mL) 16 μL | pcDNA3.1(+)CH-HSP27(1 mg/mL) 4 μL | 980 | 1000 |

*OptiPRO (TM) SFM (Cat. No. 12309050)

Within 18 to 22 hours after transfecting with the plasmid vectors, ExpiFectamine™ CHO Enhancer (150 μL) and ExpiCHO™ Feed (4 mL) were added to each of the culture solutions, and culture was performed at 32° C., 5% CO₂, and 125 rpm. On day 5 of culture, ExpiCHO™ Feed (4 mL) was further added to each of the culture solutions, and culture was performed at 32° C., 5% CO₂, and 125 rpm. Between day 7 and day 13 of culture, the culture supernatants were collected, and the productivities for the cysteine knot protein (hBDNF-Fc fusion protein) were calculated through ELISA. The cell survival rates were calculated through measurement of total cell counts and viable cell counts by using a Countess II FL automatic cell counter (Cat. No. AMQAF1000, ThermoFisher Scientific K.K.).

The hBDNF-Fc fusion protein concentrations of the culture supernatants were calculated by using ELISA (Biosensis Pty Ltd., Cat. No. BEK-2211-1P/2P). On day 12 of culture, cells were collected, and the viable cell counts were determined by using a Countess II FL automatic cell counter, centrifugation was performed at 10,000×g for 5 minutes, and the culture supernatants were then collected. The culture supernatants collected were diluted to a degree that allowed quantification with standards (7.8 to 500 pg/mL) (dilution rate: 100,000-fold to 1,000,000-fold). To wells of a microplate, standards, a QC sample included in the kit, and the diluted culture supernatants each in 100 μL were added, and stirring was performed at room temperature for 45 minutes. Thereafter, the samples were removed from the wells, and the wells were washed five times with a washing solution (200 μL/well×5 times). A detection antibody was added at 100 μL/well, and stirring was performed at room temperature for 30 minutes. The antibody was removed from the wells, and washing was performed in the same manner. TMB reagent was added to the wells for reaction, and, after finding the occurrence of reaction with moderate blue coloring, a stop solution was added to the wells. The absorbances at a wavelength of 450 nm were measured by using a microplate reader (SpectraMax M5e, Molecular Devices LLC.), and the production levels (concentrations) of hBDNF-Fc fusion protein in the culture supernatants were the presence or absence of expression-enhancing factors (Table 8-2). The results revealed production-enhancing effect for the hBDNF-Fc fusion proteins of all types of HSP. Co-expression of HSP90α and CDC37 gave a hBDNF-Fc fusion protein production level higher than that for single expression of HSP90α (sample Nos. 8-2, 8-8).

TABLE 8-2

| | | hBDNF-Fc | Total | Viable | | Production |
|---|---|---|---|---|---|---|
| | | production | cell | cell | Viable | level |
| Sample | Gene used for transfection | level | count | count | cell | per cell |
| No. | (+hBDNF-Fc) | (μg/mL) | (cells/mL) | (cells/mL) | rate | (pg/cell) |
| 8-1 | EGFP (control) | 0.32 | 1.4 × 10⁷ | 4.3 × 10⁶ | 31% | 0.07 |
| 8-2 | hHSP90AA1_NP_001017963 | 1.44 | 1.6 × 10⁷ | 5.1 × 10⁶ | 31% | 0.29 |
| 8-3 | hHSP90AA1_NP_005339 | 1.02 | 2.0 × 10⁷ | 9.0 × 10⁶ | 45% | 0.11 |
| 8-4 | hHSP90AB1_NP_001258899 | 1.16 | 1.5 × 10⁷ | 6.4 × 10⁶ | 43% | 0.18 |
| 8-5 | hHSP90AB1_NP_001258900 | 0.69 | 1.4 × 10⁷ | 6.0 × 10⁶ | 42% | 0.12 |
| 8-6 | hHSP90AB1_NP_001258901 | 1.81 | 1.4 × 10⁷ | 4.9 × 10⁶ | 35% | 0.37 |
| 8-7 | hCDC37 | 1.67 | 1.3 × 10⁷ | 4.8 × 10⁶ | 37% | 0.35 |
| 8-8 | hHSP90AA1_NP_001017963/hCDC37 | 1.60 | 1.7 × 10⁷ | 5.9 × 10⁶ | 35% | 0.27 |
| 8-9 | CH-HSP90AA1 | 1.10 | 1.9 × 10⁷ | 5.9 × 10⁶ | 31% | 0.19 |
| 8-10 | CH-HSP90AB1 | 0.63 | 2.1 × 10⁷ | 8.5 × 10⁶ | 41% | 0.07 |
| 8-11 | CH-CDC37 | 0.99 | 1.6 × 10⁷ | 6.7 × 10⁶ | 41% | 0.15 |
| 8-12 | hHSP10 | 0.89 | 1.4 × 10⁷ | 5.4 × 10⁶ | 38% | 0.17 |
| 8-13 | hHSP60 | 0.74 | 1.9 × 10⁷ | 7.3 × 10⁶ | 39% | 0.10 |
| 8-14 | hHSP110 | 0.89 | 1.8 × 10⁷ | 7.2 × 10⁶ | 40% | 0.12 |
| 8-15 | CH-HSP70 | 1.72 | 1.9 × 10⁷ | 6.7 × 10⁶ | 35% | 0.26 |
| 8-16 | CH-HSP27 | 1.73 | 2.1 × 10⁷ | 5.5 × 10⁶ | 27% | 0.31 |

<<Examination of Enhancing Effects of Expression-Enhancing Factors in Production of hGDNF-Fc Fusion Protein by Using Expi-CHO Expression System>>

The following operations were performed by using a Gibco™ Expi™ Expression System (Cat. No. A29133, ThermoFisher Scientific K.K.) in accordance with a Max Titer protocol. First, cultured Expi-CHO cells (6×10⁶ cells/mL) were added to a 125-mL Erlenmeyer flask (Corning Inc. Cat. No. 431143) containing ExpiCHO™ Expression Medium (Cat. No. A29100-01, ThermoFisher Scientific K.K.) (25 mL). Next, a reagent (1 ml) containing plasmid vectors shown in Table 9-1 below was prepared. Separately from the tube for the reagent containing plasmid vectors, Expifectamine (Cat. No. A12129) (80 μL) and OptiPRO™ SFM (Cat. No. 12309050) (920 μL) were added to another tube. The reagent containing plasmid vectors and the reagent containing Expifectamine were each stirred, and left to stand at room temperature for 1 to 5 minutes. Thereafter, the two reagents were slowly mixed together to form ExpiFectamine™ CHO/plasmid DNA complexes, which were left to stand at room temperature for 1 to 5 minutes. The complexes were added to the 125-mL Erlenmeyer flask containing Expi-CHO cells, and stirring culture was performed at 37° C., 8% CO₂, and 125 rpm overnight.

TABLE 9-1

| | | | | Total |
|---|---|---|---|---|
| Sample | Cysteine knot protein | Chaperone protein or control | SFM* | volume |
| No. | (plasmid name) | (plasmid name) | (μL) | (μL) |
| 9-1 | pcDNA3.1(+)hGDNF-Fc (1 mg/mL) 16 μL | pcDNA3.1(+)EGFP (1 mg/mL) 4 μL | 980 | 1000 |
| 9-2 | pcDNA3.1(+)hGDNF-Fc (1 mg/mL) 16 μL | pcDNA3.1(+)hHSP90AA1_NP_001017963 (1 mg/mL) 4 μL | 980 | 1000 |
| 9-3 | pcDNA3.1(+)hGDNF-Fc (1 mg/mL) 16 μL | pcDNA3.1(+)hHSP90AA1_NP_005339 (1 mg/mL) 4 μL | 980 | 1000 |
| 9-4 | pcDNA3.1(+)hGDNF-Fc (1 mg/mL) 16 μL | pcDNA3.1(+)hHSP90AB1_NP_001258899 (1 mg/mL) 4 μL | 980 | 1000 |
| 9-5 | pcDNA3.1(+)hGDNF-Fc (1 mg/mL) 16 μL | pcDNA3.1(+)hHSP90AB1_NP_001258900 (1 mg/mL) 4 μL | 980 | 1000 |
| 9-6 | pcDNA3.1(+)hGDNF-Fc (1 mg/mL) 16 μL | pcDNA3.1(+)hHSP90AB1_NP_001258901 (1 mg/mL) 4 μL | 980 | 1000 |
| 9-7 | pcDNA3.1(+)hGDNF-Fc (1 mg/mL) 16 μL | pcDNA3.1(+)hCDC37 (1 mg/mL) 4 μL | 980 | 1000 |
| 9-8 | pcDNA3.1(+)hGDNF-Fc (1 mg/mL) 16 μL | pcDNA3.1(+)hHSP90AA1_NP_001017963 (1 mg/mL) 2 μL | 980 | 1000 |
| | | pcDNA3.1(+)hCDC37 (1 mg/mL) 2 μL | | |
| 9-9 | pcDNA3.1(+)hGDNF-Fc (1 mg/mL) 16 μL | pcDNA3.1(+)CH-HSP90AA1 (1 mg/mL) 4 μL | 980 | 1000 |
| 9-10 | pcDNA3.1(+)hGDNF-Fc (1 mg/mL) 16 μL | pcDNA3.1(+)CH-HSP90AB1 (1 mg/mL) 4 μL | 980 | 1000 |
| 9-11 | pcDNA3.1(+)hGDNF-Fc (1 mg/mL) 16 μL | pcDNA3.1(+)CH-CDC37 (1 mg/mL) 4 μL | 980 | 1000 |
| 9-12 | pcDNA3.1(+)hGDNF-Fc (1 mg/mL) 16 μL | pcDNA3.1(+)hHSP10 (1 mg/mL) 4 μL | 980 | 1000 |
| 9-13 | pcDNA3.1(+)hGDNF-Fc (1 mg/mL) 16 μL | pcDNA3.1(+)hHSP60 (1 mg/mL) 4 μL | 980 | 1000 |
| 9-14 | pcDNA3.1(+)hGDNF-Fc (1 mg/mL) 16 μL | pcDNA3.1(+)hHSP110 (1 mg/mL) 4 μL | 980 | 1000 |
| 9-15 | pcDNA3.1(+)hGDNF-Fc (1 mg/mL) 16 μL | pcDNA3.1(+)CH-HSP70 (1 mg/mL) 4 μL | 980 | 1000 |
| 9-16 | pcDNA3.1(+)hGDNF-Fc (1 mg/mL) 16 μL | pcDNA3.1(+)CH-HSP27(1 mg/mL) 4 μL | 980 | 1000 |
| 9-17 | pcDNA3.1(+)hGDNF-Fc (1 mg/mL) 16 μL | pcDNA3.1(+)hHSP40 (1 mg/mL) 4 μL | 980 | 1000 |

*OptiPRO (TM) SFM (Cat. No. 12309050)

Within 18 to 22 hours after transfecting with the plasmid vectors, ExpiFectamine™ CHO Enhancer (150 μL) and ExpiCHO™ Feed (4 mL) were added to each of the culture solutions, and culture was performed at 32° C., 5% CO₂, and 125 rpm. On day 5 of culture, ExpiCHO™ Feed (4 mL) was further added to each of the culture solutions, and culture was performed at 32° C., 5% CO₂, and 125 rpm. Between day 7 and day 13 of culture, the culture supernatants were collected, and the productivities for the cysteine knot protein (hGDNF-Fc fusion protein) were calculated through ELISA. The cell survival rates were calculated through measurement of total cell counts and viable cell counts by using a Countess II FL automatic cell counter (Cat. No. AMQAF1000, ThermoFisher Scientific K.K.).

The hGDNF-Fc fusion protein concentrations of the culture supernatants were calculated by using ELISA (Biosensis Pty Ltd., Cat. No. BEK-2211-1P/2P). On day 12 of culture, cells were collected, and the viable cell counts were determined by using a Countess II FL automatic cell counter, centrifugation was performed at 10,000×g for 5 minutes, and the culture supernatants were then collected. The culture supernatants collected were diluted to a degree that allowed quantification with standards (7.8 to 500 pg/mL) (dilution rate: 10,000-fold to 100,000-fold). To wells of a microplate, standards, a QC sample included in the kit, and the diluted culture supernatants each in 100 μL were added, and stirring was performed at room temperature for 45 minutes. Thereafter, the samples were removed from the wells, and the wells were washed five times with a washing solution (200 μL/well×5 times). A detection antibody was added at 100 μL/well, and stirring was performed at room temperature for 30 minutes. The antibody was removed from the wells, and washing was performed in the same manner. TMB reagent was added to the wells for reaction, and, after finding the occurrence of reaction with moderate blue coloring, a stop solution was added to the wells. The absorbances at a wavelength of 450 nm were measured by using a microplate reader (SpectraMax M5e, Molecular Devices LLC.), and the production levels (concentrations) of hGDNF-Fc fusion protein in the culture supernatants were the presence or absence of expression-enhancing factors (Table 9-2). The results revealed production-enhancing effect for the hGDNF-Fc fusion proteins of all types of HSP.

TABLE 9-2

| | | hGDNF-Fc | Total | Viable | | Production |
|---|---|---|---|---|---|---|
| | | production | cell | cell | Viable | level |
| Sample | Gene used for transfection | level | count | count | cell | per cell |
| No | (+hGDNF-Fc) | (μg/mL) | (cells/mL) | (cells/mL) | rate | (pg/cell) |
| 9-1 | EGFP (control) | 0.24 | 1.3 × 10⁷ | 3.1 × 10⁶ | 23.6% | 0.08 |
| 9-2 | hHSP90AA1_NP_001017963 | 0.72 | 1.6 × 10⁷ | 4.4 × 10⁶ | 27.5% | 0.16 |
| 9-3 | hHSP90AA1_NP_005339 | 0.82 | 1.6 × 10⁷ | 4.5 × 10⁶ | 27.5% | 0.18 |
| 9-4 | hHSP90AB1_NP_001258899 | 1.26 | 1.6 × 10⁷ | 4.8 × 10⁶ | 29.5% | 0.26 |
| 9-5 | hHSP90AB1_NP_001258900 | 0.95 | 1.5 × 10⁷ | 3.8 × 10⁶ | 25.5% | 0.25 |
| 9-6 | hHSP90AB1_NP_001258901 | 0.5 | 1.7 × 10⁷ | 3.9 × 10⁶ | 23.5% | 0.13 |
| 9-7 | hCDC37 | 0.6 | 1.5 × 10⁷ | 4.2 × 10⁶ | 28% | 0.14 |
| 9-8 | hHSP90AA1_NP_001017963/hCDC37 | 0.43 | 1.5 × 10⁷ | 4.0 × 10⁶ | 26.5% | 0.11 |
| 9-9 | CH-HSP90AA1 | 1.3 | 2.0 × 10⁷ | 5.5 × 10⁶ | 27.5% | 0.24 |
| 9-10 | CH-HSP90AB1 | 0.78 | 2.0 × 10⁷ | 6.0 × 10⁶ | 30.6% | 0.13 |
| 9-11 | CH-CDC37 | 0.96 | 1.9 × 10⁷ | 4.3 × 10⁶ | 23% | 0.22 |
| 9-12 | hHSP10 | 0.81 | 1.9 × 10⁷ | 5.1 × 10⁶ | 27% | 0.16 |
| 9-13 | hHSP60 | 0.81 | 1.7 × 10⁷ | 3.9 × 10⁶ | 23.5% | 0.21 |
| 9-14 | hHSP110 | 0.79 | 1.5 × 10⁷ | 3.9 × 10⁶ | 25.5% | 0.2 |
| 9-15 | CH-HSP70 | 0.7 | 1.5 × 10⁷ | 3.5 × 10⁶ | 24% | 0.2 |
| 9-16 | CH-HSP27 | 0.63 | 2.0 × 10⁷ | 5.8 × 10⁶ | 29% | 0.11 |
| 9-17 | hHSP40 | 1.11 | 1.5 × 10⁷ | 3.8 × 10⁶ | 24.5% | 0.29 |

Although embodiments and examples of the present invention have been described as above, appropriate combinations of the configurations of the embodiments and examples described above have been originally contemplated.

The embodiments and examples disclosed herein are exemplary in all respects, and should not be construed to be restrictive. The scope of the present invention is specified not by the above embodiments and examples but by claims, and modifications equivalent in meaning to claims and those within the scope of claims are all intended to be included.

1. A method for producing a cysteine knot protein, the method comprising:
producing the cysteine knot protein by culturing a transformed mammalian cell containing a gene encoding the cysteine knot protein and a gene encoding an exogenous chaperone protein in a protein production medium; and
collecting the produced cysteine knot protein, wherein
the chaperone protein includes one or more selected from the group consisting of HSP90α, HSP90β, CDC37, HSP70, HSP40, HSP60, HSP10, HSP110, and HSP27.

2. The method for producing a cysteine knot protein according to claim 1, the method comprising:
providing a mammalian cell;
transforming the mammalian cell with a gene encoding the cysteine knot protein and a gene encoding the chaperone protein;
producing the cysteine knot protein by culturing the transformed mammalian cell in a protein production medium; and
collecting the produced cysteine knot protein, wherein
the chaperone protein includes one or more selected from the group consisting of HSP90α, HSP90β, CDC37, HSP70, HSP40, HSP60, HSP10, HSP110, and HSP27.

3. The method for producing a cysteine knot protein according to claim 2, wherein the transforming the mammalian cell is performed with one or more recombinant protein expression vectors each containing a gene encoding the cysteine knot protein and a gene encoding the chaperone protein.

4. The method for producing a cysteine knot protein according to claim 2, wherein the transforming the mammalian cell is performed by simultaneously or separately bringing one or more recombinant protein expression vectors each containing a gene encoding the cysteine knot protein and one or more expression-enhancing vectors each containing a gene encoding the chaperone protein into contact with the mammalian cell.

5. The method for producing a cysteine knot protein according to claim 1, the method comprising:
providing a mammalian cell containing one or more recombinant protein expression vectors each containing a gene encoding the cysteine knot protein;
transforming the mammalian cell with at least one expression-enhancing vector containing a gene encoding the chaperone protein;
producing the cysteine knot protein by culturing the transformed mammalian cell in a protein production medium; and
collecting the produced cysteine knot protein, wherein
the chaperone protein includes one or more selected from the group consisting of HSP90α, HSP90β, CDC37, HSP70, HSP40, HSP60, HSP10, HSP110, and HSP27.

6. The method for producing a cysteine knot protein according to claim 5, wherein
the expression-enhancing vector includes a first expression-enhancing vector containing a gene encoding a first chaperone protein and a second expression-enhancing vector containing a gene encoding a second chaperone protein, and
the first chaperone protein is different from the second chaperone protein.

7. The method for producing a cysteine knot protein according to claim 1, wherein the chaperone protein includes either one or both of HSP90α and CDC37.

8. The method for producing a cysteine knot protein according to claim 1, wherein
the cysteine knot protein has a cysteine knot motif having two or more cysteine residues, and
the two or more cysteine residues forms one or more intramolecular disulfide bonds.

9. The method for producing a cysteine knot protein according to claim 1, wherein the cysteine knot protein includes one or more selected from the group consisting of neurotrophins, proteins belonging to a PDGF like super family, proteins belonging to a TGFβ super family, coagulogen, noggin, IL-17F, proteins belonging to a thyroid stimulating hormone family, and proteins belonging to a gonadotropic hormone family.

10. The method for producing a cysteine knot protein according to claim 1, wherein the cysteine knot protein includes one or more selected from the group consisting of BDNF, NT3, PDGF-β, GDNF, IL-17F, and NGF.

11. The method for producing a cysteine knot protein according to claim 1, wherein the mammalian cell includes one or more selected from the group consisting of a CHO cell, a COS cell, a BHK cell, a HeLa cell, an HEK293 cell, an NS0 cell, and an Sp2/0 cell.

12. A mammalian cell for recombinant protein production, the mammalian cell comprising one or more recombinant protein expression vectors each containing a gene encoding a cysteine knot protein, wherein
the mammalian cell for recombinant protein production further comprises one or more expression-enhancing vectors each containing a gene encoding a chaperone protein, and
the chaperone protein includes one or more selected from the group consisting of HSP90α, HSP90β, CDC37, HSP70, HSP40, HSP60, HSP10, HSP110, and HSP27.

13. A kit for enhancing a production of a cysteine knot protein in a mammalian cell, wherein
the kit comprises one or more expression-enhancing vectors each containing a gene encoding a chaperone protein, and
the chaperone protein includes at least one selected from the group consisting of HSP90α, HSP90β, CDC37, HSP70, HSP40, HSP60, HSP10, HSP110, and HSP27.


SEQUENCE LISTING (100 sequences)

SEQ ID NO: 1 - DNA, 2583 bp, Artificial Sequence
  codon-optimized gene of NP_001017963 (hHSP90 alpha)
  CDS (13)..(2577)
aagcttgccaccatgcccccttgcagcggcggcgacggctctaccccacccggaccaagcctgagagatcgcgactgtccagcccagtctgctgagtatcccagggatcggctggacccaaggcccggctctccctctgaggcctccagccctccattcctgagatcccgcgctcctgtgaactggtatcaggagaaggcccaggtgtttctgtggcacctgatggtgagcggctctaccacactgctgtgcctgtggaagcagcctttccatgtgagcgcttttccagtgaccgcctccctggctttcaggcagtctcagggagctggacagcacctgtacaaggatctgcagccctttatcctgctgcggctgctgatgcctgaggagacacagacacaggaccagccaatggaggaggaggaggtggagacattcgcctttcaggctgagatcgcccagctgatgtctctgatcatcaacacattctactccaataaggagatctttctgagggagctgatcagcaattcttccgatgccctggacaagatccggtatgagtctctgaccgatccatctaagctggactccggcaaggagctgcacatcaacctgatccccaataagcaggatcggaccctgacaatcgtggacaccggcatcggcatgacaaaggctgacctgatcaacaatctgggcaccatcgccaagagcggcacaaaggctttcatggaggccctgcaggctggagctgatatctccatgatcggccagttcggcgtgggcttttacagcgcttatctggtggccgagaaggtgaccgtgatcacaaagcataacgacgatgagcagtacgcttgggagagctctgccggcggctcttttaccgtgaggaccgatacaggcgagcctatgggccggggcaccaaagtgatcctgcacctgaaggaggaccagacagagtacctggaggagaggcggatcaaggagatcgtgaagaagcattcccagttcatcggctatccaatcacactgtttgtggagaaggagagggataaggaggtgagcgacgatgaggctgaggagaaggaggacaaggaggaggagaaggagaaggaggagaaggagtctgaggacaagcccgagatcgaggatgtgggctccgacgaggaggaggagaagaaggatggcgacaagaagaagaagaagaagatcaaggagaagtatatcgatcaggaggagctgaataagaccaagcccatctggacacggaaccctgacgatatcaccaatgaggagtacggcgagttctataagtccctgacaaacgattgggaggaccacctggccgtgaagcattttagcgtggagggccagctggagttcagagctctgctgtttgtgcctagacgcgccccattcgacctgtttgagaataggaagaagaagaacaatatcaagctgtacgtgaggcgggtgttcatcatggataactgcgaggagctgatccctgagtatctgaattttatcagaggcgtggtggattctgaggacctgccactgaatatcagcagagagatgctgcagcagtctaagatcctgaaagtgatccgcaagaacctggtgaagaagtgtctggagctgttcaccgagctggctgaggacaaggagaactacaagaagttctatgagcagttttccaagaatatcaagctgggcatccacgaggattcccagaacaggaagaagctgagcgagctgctgcggtactatacctccgccagcggcgatgagatggtgagcctgaaggactactgtacacgcatgaaggagaatcagaagcacatctactatatcaccggcgagacaaaggaccaggtggctaactctgccttcgtggagaggctgcggaagcatggcctggaagtgatctacatgatcgagccaatcgatgagtattgcgtgcagcagctgaaggagtttgagggcaagaccctggtgtccgtgacaaaggagggcctggagctgcctgaggatgaggaggagaagaagaagcaggaggagaagaagaccaagttcgagaatctgtgtaagatcatgaaggatatcctggagaagaaggtggagaaggtggtggtgtctaacagactggtgacctccccctgctgtatcgtgacctctacatacggctggacagctaatatggagagaatcatgaaggcccaggctctgcgcgataactccaccatgggctatatggccgctaagaagcacctggagatcaatcctgaccattctatcatcgagacactgcgccagaaggccgaggctgataagaacgacaagtccgtgaaggacctggtcatcctgctgtacgagacagctctgctgtccagcggctttagcctggaggacccccagacacatgccaacagaatctatcgcatgatcaagctgggcctgggcatcgatgaggacgatcctaccgccgacgatacatctgctgctgtgacagaggagatgccccctctggagggcgacgatgacacctccaggatggaggaggtggactgagaattc

SEQ ID NO: 2 - protein, 854 aa, Artificial Sequence
  Synthetic Construct
MPPCSGGDGSTPPGPSLRDRDCPAQSAEYPRDRLDPRPGSPSEASSPPFLRSRAPVNWYQEKAQVFLWHLMVSGSTTLLCLWKQPFHVSAFPVTASLAFRQSQGAGQHLYKDLQPFILLRLLMPEETQTQDQPMEEEEVETFAFQAEIAQLMSLIINTFYSNKEIFLRELISNSSDALDKIRYESLTDPSKLDSGKELHINLIPNKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTADDTSAAVTEEMPPLEGDDDTSRMEEVD

SEQ ID NO: 3 - DNA, 2217 bp, Artificial Sequence
  codon-optimized gene of NP_005339 (hHSP90 alpha)
  CDS (13)..(2211)
aagcttgccaccatgcccgaggagacccagacacaggaccagcctatggaggaggaggaggtggagaccttcgcctttcaggctgagatcgcccagctgatgagcctgatcatcaacacattctactctaataaggagatctttctgagagagctgatcagcaattccagcgatgccctggacaagatccgctatgagtctctgaccgatccatctaagctggactccggcaaggagctgcacatcaacctgatccccaataagcaggataggaccctgacaatcgtggacaccggcatcggcatgacaaaggctgacctgatcaacaatctgggcaccatcgccaagtctggcacaaaggctttcatggaggccctgcaggctggagctgatatctccatgatcggccagttcggcgtgggcttttactccgcctatctggtggccgagaaggtgaccgtgatcacaaagcataacgacgatgagcagtacgcttgggagtcttccgccggcggctcctttaccgtgagaaccgatacaggcgagcctatgggccgcggcaccaaagtgatcctgcacctgaaggaggaccagacagagtacctggaggagaggcggatcaaggagatcgtgaagaagcattcccagttcatcggctatccaatcaccctgtttgtggagaaggagagggataaggaggtgagcgacgatgaggctgaggagaaggaggacaaggaggaggagaaggagaaggaggagaaggagtctgaggacaagccagagatcgaggatgtgggctccgacgaggaggaggagaagaaggatggcgacaagaagaagaagaagaagatcaaggagaagtatatcgatcaggaggagctgaataagaccaagcctatctggacacggaacccagacgatatcaccaatgaggagtacggcgagttctataagtccctgacaaacgattgggaggaccacctggccgtgaagcacttctccgtggagggccagctggagttcagggctctgctgtttgtgccaagacgcgcccccttcgacctgtttgagaacaggaagaagaagaacaacatcaagctgtacgtgaggcgggtgttcatcatggataactgcgaggagctgatccctgagtatctgaacttcatcagaggcgtggtggattccgaggacctgccactgaatatctccagggagatgctgcagcagagcaagatcctgaaagtgatccggaagaacctggtgaagaagtgtctggagctgttcaccgagctggctgaggacaaggagaactacaagaagttctatgagcagtttagcaagaatatcaagctgggcatccacgaggattctcagaacagaaagaagctgtccgagctgctgcgctactatacctccgccagcggcgatgagatggtgagcctgaaggactactgcacacgcatgaaggagaatcagaagcacatctactatatcaccggcgagacaaaggaccaggtggctaactctgccttcgtggagagactgcgcaagcatggcctggaagtgatctacatgatcgagccaatcgatgagtattgcgtgcagcagctgaaggagtttgagggcaagaccctggtgagcgtgacaaaggagggactggagctgccagaggacgaggaggagaagaagaagcaggaggagaagaagaccaagttcgagaacctgtgcaagatcatgaaggatatcctggagaagaaggtggagaaggtggtggtgagcaacagactggtgacctctccctgctgtatcgtgacctctacatacggctggacagctaatatggagaggatcatgaaggcccaggctctgcgggataactccaccatgggctatatggccgctaagaagcacctggagatcaatcctgaccattctatcatcgagacactgcgccagaaggccgaggctgataagaacgacaagtccgtgaaggacctggtcatcctgctgtacgagaccgctctgctgagctctggcttttctctggaggacccccagacacatgccaacaggatctatcggatgatcaagctgggcctgggcatcgatgaggacgatcctaccgccgacgatacaagcgccgctgtgaccgaggagatgccacctctggagggcgacgatgacacatctagaatggaggaggtggactgagaattc

SEQ ID NO: 4 - protein, 732 aa, Artificial Sequence
  Synthetic Construct
MPEETQTQDQPMEEEEVETFAFQAEIAQLMSLIINTFYSNKEIFLRELISNSSDALDKIRYESLTDPSKLDSGKELHINLIPNKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTADDTSAAVTEEMPPLEGDDDTSRMEEVD

SEQ ID NO: 5 - DNA, 2220 bp, Artificial Sequence
  codon-optimized gene of NP_001233750 (chHSP90 alpha)
  CDS (13)..(2214)
aagcttgccaccatgcccgaggagacccagacacaggaccagcctatggaggaggaggaggtggagaccttcgcctttcaggctgagatcgcccagctgatgagcctgatcatcaacacattctactctaataaggagatctttctgagagagctgatctccaattccagcgatgctctggacaagatccgctatgagagcctgaccgatccatctaagctggactccggcaaggagctgcacatcaacatcatccccaataagcaggataggaccctgacaatcgtggacaccggcatcggcatgacaaaggctgacctgatcaacaatctgggcaccatcgccaagagcggcacaaaggctttcatggaggccctgcaggctggagctgatatctctatgatcggccagttcggcgtgggcttttacaccgcttatctggtggccgagaaggtgaccgtgatcacaaagcataacgacgatgagcagtacgcttgggagtcttccgccggaggctcctttaccgtgaggaccgatacaggcgagcctatgggccggggcaccaaagtgatcctgcacctgaaggaggaccagacagagtacatggaggagaggcggatcaaggagatcgtgaagaagcatagccagttcatcggctatccaatcaccctgtttgtggagaaggagagagataaggaggtgtctgacgatgaggctgaggagaaggaggacaaggaggaggagaaggagaaggaggagaagggcatcgacgataagccagagatcgaggatgtgggcagcgacgaggaggaggaggagaagaaggatggcgacaagaagaagaagaagaagatcaaggagaagtacatcgaccaggaggagctgaataagaccaagcctatctggacacgcaacccagacgatatcaccaatgaggagtacggcgagttctataagtctctgacaaacgattgggaggagcacctggccgtgaagcatttttccgtggagggccagctggagttcagagctctgctgtttgtgccaagacgcgcccccttcgacctgtttgagaacaggaagaagaagaacaacatcaagctgtacgtgaggcgggtgttcatcatggataactgcgaggagctgttccctgagtatctgaacttcatcaggggcgtggtggatagcgaggacctgccactgaatatctcccgggagatcctgcagcagagcaagatcctgaaagtgatcagaaagaacctggtgcgcaagtgtctggagctgtttcacgagctggctgaggacaaggagaactacaagaagttctatgagcagttttctaagaatatcaagctgggcatccatgaggattctcagaacaggaagaagctgtccgagctgctgcggtactatacctccgccagcggcgatgagatggtgtctctgaaggactactgcacaaggatgaaggagaatcagaagcacatctatttcatcaccggcgagacaaaggaccaggtggctaactccgcctttgtggagagactgcgcaagcatggcctggaagtgatctacatgatcgagccaatcgatgagtattgcgtgcagcagctgaaggagttcgagggcaagaccctggtgtctgtgacaaaggagggactggagctgccagaggacgaggaggagaagaagaagcaggaggagaagaagaccaagtttgagaacctgtgcaagatcatgaaggatatcctggagaagaaggtggagaaggtggtggtgagcaacaggctggtgacctctccctgctgtatcgtgacctccacatacggctggacagccaatatggagagaatcatcaaggcccaggctctgcgcgataacagcaccatgggctatatggccgctaagaagcacctggagatcaatcctgaccattccatcatcgagacactgcggcagaaggccgaggctgataagaacgacaagagcgtgaaggacctggtcatcctgctgtacgagaccgctctgctgagctctggcttctccctggaggacccccagacacacgccaacaggatctatcggatgatcaagctgggcctgggcatcgatgaggacgatcctaccgtggacgatacatctgccgctgtgaccgaggagatgccacctctggagggcgacgatgacacatccagaatggaggaggtggactgagaattc

SEQ ID NO: 6 - protein, 733 aa, Artificial Sequence
  Synthetic Construct
MPEETQTQDQPMEEEEVETFAFQAEIAQLMSLIINTFYSNKEIFLRELISNSSDALDKIRYESLTDPSKLDSGKELHINIIPNKQDRTLTIVDTGIGMTKADLINNLGTIAKSGTKAFMEALQAGADISMIGQFGVGFYTAYLVAEKVTVITKHNDDEQYAWESSAGGSFTVRTDTGEPMGRGTKVILHLKEDQTEYMEERRIKEIVKKHSQFIGYPITLFVEKERDKEVSDDEAEEKEDKEEEKEKEEKGIDDKPEIEDVGSDEEEEEKKDGDKKKKKKIKEKYIDQEELNKTKPIWTRNPDDITNEEYGEFYKSLTNDWEEHLAVKHFSVEGQLEFRALLFVPRRAPFDLFENRKKKNNIKLYVRRVFIMDNCEELFPEYLNFIRGVVDSEDLPLNISREILQQSKILKVIRKNLVRKCLELFHELAEDKENYKKFYEQFSKNIKLGIHEDSQNRKKLSELLRYYTSASGDEMVSLKDYCTRMKENQKHIYFITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIIKAQALRDNSTMGYMAAKKHLEINPDHSIIETLRQKAEADKNDKSVKDLVILLYETALLSSGFSLEDPQTHANRIYRMIKLGLGIDEDDPTVDDTSAAVTEEMPPLEGDDDTSRMEEVD

SEQ ID NO: 7 - DNA, 2193 bp, Artificial Sequence
  codon-optimized gene of NP_001258899 (hHSP90 beta)
  CDS (13)..(2187)
aagcttgccaccatgcccgaggaggtgcaccatggcgaggaggaggtggagaccttcgcctttcaggctgagatcgcccagctgatgtccctgatcatcaacacattctacagcaataaggagatctttctgagggagctgatcagcaatgcttctgacgccctggataagatccggtatgagtccctgaccgacccttctaagctggattccggcaaggagctgaagatcgacatcatcccaaacccccaggagaggaccctgacactggtggacaccggcatcggcatgacaaaggctgatctgatcaacaatctgggcaccatcgccaagagcggcacaaaggctttcatggaggccctgcaggctggagctgatatctccatgatcggccagttcggcgtgggcttttactccgcctatctggtggccgagaaggtggtggtcatcaccaagcacaacgacgatgagcagtacgcttgggagtccagcgccggaggcagcttcacagtgagggctgaccacggagagccaatcggaaggggcaccaaagtgatcctgcatctgaaggaggatcagacagagtatctggaggagaggcgggtgaaggaggtggtgaagaagcatagccagtttatcggctaccctatcaccctgtatctggagaaggagcgggagaaggagatctctgacgatgaggccgaggaggagaagggcgagaaggaggaggaggacaaggacgatgaggagaagccaaagatcgaggacgtgggcagcgatgaggaggacgattctggcaaggacaagaagaagaagaccaagaagatcaaggagaagtacatcgatcaggaggagctgaacaagaccaagcctatctggacaaggaatccagacgatatcacccaggaggagtacggcgagttctataagagcctgacaaatgactgggaggatcacctggccgtgaagcatttttctgtggagggccagctggagttccgggctctgctgtttatccccagacgcgcccctttcgacctgtttgagaacaagaagaagaagaacaacatcaagctgtacgtgaggcgggtgttcatcatggactcttgcgatgagctgatccccgagtatctgaacttcatcagaggcgtggtggactccgaggatctgcctctgaacatctccagagagatgctgcagcagagcaagatcctgaaagtgatccgcaagaatatcgtgaagaagtgtctggagctgttttccgagctggctgaggacaaggagaactacaagaagttctatgaggcctttagcaagaatctgaagctgggcatccacgaggactctaccaacagacgcaggctgtccgagctgctgagataccatacctcccagagcggcgatgagatgacatctctgtccgagtacgtgtcccgcatgaaggagacccagaagtccatctactatatcacaggcgagagcaaggagcaggtggctaactctgccttcgtggagcgggtgagaaagcgcggctttgaggtggtgtacatgaccgagccaatcgacgagtattgcgtgcagcagctgaaggagttcgatggcaagtccctggtgagcgtgacaaaggagggactggagctgccagaggacgaggaggagaagaagaagatggaggagtctaaggctaagtttgagaacctgtgcaagctgatgaaggagatcctggataagaaggtggagaaggtgaccatctctaacagactggtgtcttccccatgctgtatcgtgacctccacatacggctggaccgctaatatggagagaatcatgaaggcccaggctctgcgcgacaactccacaatgggctatatgatggccaagaagcacctggagatcaatcctgatcatccaatcgtggagaccctgaggcagaaggctgaggctgacaagaacgataaggctgtgaaggacctggtggtgctgctgttcgagaccgccctgctgagctctggctttagcctggaggatccccagacacactctaataggatctaccggatgatcaagctgggcctgggcatcgacgaggatgaggtggctgctgaggagccaaacgctgctgtgccagacgagatcccacctctggagggcgacgaggatgcttctcgcatggaggaggtggattgagaattc

SEQ ID NO: 8 - protein, 724 aa, Artificial Sequence
  Synthetic Construct
MPEEVHHGEEEVETFAFQAEIAQLMSLIINTFYSNKEIFLRELISNASDALDKIRYESLTDPSKLDSGKELKIDIIPNPQERTLTLVDTGIGMTKADLINNLGTIAKSGTKAFMEALQAGADISMIGQFGVGFYSAYLVAEKVVVITKHNDDEQYAWESSAGGSFTVRADHGEPIGRGTKVILHLKEDQTEYLEERRVKEVVKKHSQFIGYPITLYLEKEREKEISDDEAEEEKGEKEEEDKDDEEKPKIEDVGSDEEDDSGKDKKKKTKKIKEKYIDQEELNKTKPIWTRNPDDITQEEYGEFYKSLTNDWEDHLAVKHFSVEGQLEFRALLFIPRRAPFDLFENKKKKNNIKLYVRRVFIMDSCDELIPEYLNFIRGVVDSEDLPLNISREMLQQSKILKVIRKNIVKKCLELFSELAEDKENYKKFYEAFSKNLKLGIHEDSTNRRRLSELLRYHTSQSGDEMTSLSEYVSRMKETQKSIYYITGESKEQVANSAFVERVRKRGFEVVYMTEPIDEYCVQQLKEFDGKSLVSVTKEGLELPEDEEEKKKMEESKAKFENLCKLMKEILDKKVEKVTISNRLVSSPCCIVTSTYGWTANMERIMKAQALRDNSTMGYMMAKKHLEINPDHPIVETLRQKAEADKNDKAVKDLVVLLFETALLSSGFSLEDPQTHSNRIYRMIKLGLGIDEDEVAAEEPNAAVPDEIPPLEGDEDASRMEEVD

SEQ ID NO: 9 - DNA, 2049 bp, Artificial Sequence
  codon-optimized gene of NP_001258900 (hHSP90 beta)
  CDS (13)..(2043)
aagcttgccaccatgcctgaggaggtgcaccatggcgaggaggaggtggagaccttcgcctttcaggctgagatcgcccagctgatgtccctgatcatcaacacattctacagcaataaggagatctttctgagggagctgatcagcaatgcttctgacgccctggataagatccggtatgagtccctgaccgacccatctaagctggattccggcaaggagctgaagatcgacatctccatgatcggccagttcggcgtgggcttttactccgcctatctggtggccgagaaggtggtggtcatcaccaagcacaacgacgatgagcagtacgcttgggagtccagcgccggaggcagcttcacagtgagggctgaccacggagagccaatcggaaggggcaccaaagtgatcctgcatctgaaggaggatcagacagagtatctggaggagaggcgggtgaaggaggtggtgaagaagcatagccagtttatcggctacccaatcaccctgtatctggagaaggagagggagaaggagatctctgacgatgaggccgaggaggagaagggcgagaaggaggaggaggacaaggacgatgaggagaagcccaagatcgaggacgtgggcagcgatgaggaggacgattctggcaaggacaagaagaagaagaccaagaagatcaaggagaagtacatcgatcaggaggagctgaacaagaccaagccaatctggacaaggaatcccgacgatatcacccaggaggagtacggcgagttctataagagcctgacaaatgactgggaggatcacctggccgtgaagcatttttctgtggagggccagctggagttccgggctctgctgtttatccctagacgcgccccattcgacctgtttgagaacaagaagaagaagaacaacatcaagctgtacgtgaggcgggtgttcatcatggactcttgcgatgagctgatccctgagtatctgaacttcatcagaggcgtggtggactccgaggatctgccactgaacatctccagagagatgctgcagcagagcaagatcctgaaagtgatccgcaagaatatcgtgaagaagtgtctggagctgttctccgagctggctgaggataaggagaactacaagaagttctatgaggcctttagcaagaatctgaagctgggcatccacgaggactctaccaacagacgcaggctgtccgagctgctgagataccatacctcccagagcggcgatgagatgacatctctgtccgagtacgtgtcccgcatgaaggagacccagaagtccatctactatatcacaggcgagagcaaggagcaggtggctaactctgccttcgtggagcgggtgagaaagcgcggctttgaggtggtgtacatgaccgagcccatcgacgagtattgcgtgcagcagctgaaggagttcgatggcaagtccctggtgagcgtgacaaaggagggcctggagctgcctgaggacgaggaggagaagaagaagatggaggagtctaaggctaagtttgagaacctgtgcaagctgatgaaggagatcctggataagaaggtggagaaggtgaccatctctaacagactggtgtcttccccctgctgtatcgtgacctccacatacggctggaccgctaatatggagagaatcatgaaggcccaggctctgcgcgacaactccacaatgggctatatgatggccaagaagcacctggagatcaatccagatcatcccatcgtggagacactgcgccagaaggccgaggctgacaagaacgataaggctgtgaaggacctggtggtgctgctgttcgagaccgccctgctgagctctggctttagcctggaggatcctcagacacactctaataggatctaccggatgatcaagctgggcctgggcatcgacgaggatgaggtggctgctgaggagccaaacgctgctgtgcctgacgagatcccacctctggagggcgacgaggatgcttccaggatggaggaggtggattgagaattc

SEQ ID NO: 10 - protein, 676 aa, Artificial Sequence
  Synthetic Construct
MPEEVHHGEEEVETFAFQAEIAQLMSLIINTFYSNKEIFLRELISNASDALDKIRYESLTDPSKLDSGKELKIDISMIGQFGVGFYSAYLVAEKVVVITKHNDDEQYAWESSAGGSFTVRADHGEPIGRGTKVILHLKEDQTEYLEERRVKEVVKKHSQFIGYPITLYLEKEREKEISDDEAEEEKGEKEEEDKDDEEKPKIEDVGSDEEDDSGKDKKKKTKKIKEKYIDQEELNKTKPIWTRNPDDITQEEYGEFYKSLTNDWEDHLAVKHFSVEGQLEFRALLFIPRRAPFDLFENKKKKNNIKLYVRRVFIMDSCDELIPEYLNFIRGVVDSEDLPLNISREMLQQSKILKVIRKNIVKKCLELFSELAEDKENYKKFYEAFSKNLKLGIHEDSTNRRRLSELLRYHTSQSGDEMTSLSEYVSRMKETQKSIYYITGESKEQVANSAFVERVRKRGFEVVYMTEPIDEYCVQQLKEFDGKSLVSVTKEGLELPEDEEEKKKMEESKAKFENLCKLMKEILDKKVEKVTISNRLVSSPCCIVTSTYGWTANMERIMKAQALRDNSTMGYMMAKKHLEINPDHPIVETLRQKAEADKNDKAVKDLVVLLFETALLSSGFSLEDPQTHSNRIYRMIKLGLGIDEDEVAAEEPNAAVPDEIPPLEGDEDASRMEEVD

SEQ ID NO: 11 - DNA, 2163 bp, Artificial Sequence
  codon-optimized gene of  NP_001258901 (hHSP90 beta)
  CDS (13)..(2157)
aagcttgccaccatgcccgaggaggtgcaccatggcgaggaggaggtggagaccttcgcctttcaggctgagatcgcccagctgatgtccctgatcatcaacacattctacagcaataaggagatctttctgagggagctgatcagcaatgcttctgacgccctggataagatccggtatgagtctctgaccgacccttctaagctggattccggcaaggagctgaagatcgacatcatcccaaacccccaggagaggaccctgacactggtggacaccggcatcggcatgacaaaggctgatctgatcaacaatctgggcaccatcgccaagagcggcacaaaggctttcatggaggccctgcagttcggcgtgggcttttactctgcttatctggtggccgagaaggtggtggtcatcaccaagcacaacgacgatgagcagtacgcttgggagtccagcgccggaggctcctttacagtgagggctgaccacggagagccaatcggaaggggcaccaaagtgatcctgcatctgaaggaggatcagacagagtatctggaggagaggcgggtgaaggaggtggtgaagaagcatagccagttcatcggctaccctatcaccctgtatctggagaaggagcgggagaaggagatctctgacgatgaggccgaggaggagaagggcgagaaggaggaggaggacaaggacgatgaggagaagccaaagatcgaggacgtgggcagcgatgaggaggacgattctggcaaggacaagaagaagaagaccaagaagatcaaggagaagtacatcgatcaggaggagctgaacaagaccaagcctatctggacaaggaatccagacgatatcacccaggaggagtacggcgagttctataagtctctgacaaatgactgggaggatcacctggccgtgaagcatttttccgtggagggacagctggagttccgggctctgctgtttatccccagacgcgcccctttcgacctgtttgagaacaagaagaagaagaacaacatcaagctgtacgtgaggcgggtgttcatcatggactcttgcgatgagctgatccccgagtatctgaacttcatcagaggcgtggtggactccgaggatctgcctctgaacatctccagagagatgctgcagcagagcaagatcctgaaagtgatccgcaagaatatcgtgaagaagtgtctggagctgttttccgagctggctgaggataaggagaactacaagaagttctatgaggcctttagcaagaatctgaagctgggcatccacgaggactctaccaacagacgcaggctgtccgagctgctgagataccatacctcccagagcggcgatgagatgacatctctgtccgagtacgtgtcccgcatgaaggagacccagaagtccatctactatatcacaggcgagagcaaggagcaggtggctaactctgccttcgtggagcgggtgagaaagcgcggctttgaggtggtgtacatgaccgagccaatcgacgagtattgcgtgcagcagctgaaggagttcgatggcaagtccctggtgagcgtgacaaaggagggactggagctgccagaggacgaggaggagaagaagaagatggaggagtccaaggctaagtttgagaacctgtgcaagctgatgaaggagatcctggataagaaggtggagaaggtgaccatctccaacagactggtgtcttccccatgctgtatcgtgaccagcacatacggctggaccgctaatatggagagaatcatgaaggcccaggctctgcgcgacaacagcacaatgggctatatgatggccaagaagcacctggagatcaatcctgatcatccaatcgtggagaccctgaggcagaaggctgaggctgacaagaacgataaggctgtgaaggacctggtggtgctgctgttcgagaccgccctgctgagctctggctttagcctggaggatccccagacacactctaataggatctaccggatgatcaagctgggcctgggcatcgacgaggatgaggtggctgctgaggagccaaacgctgctgtgccagacgagatcccacctctggagggcgacgaggatgcttcccgcatggaggaggtggattgagaattc

SEQ ID NO: 12 - protein, 714 aa, Artificial Sequence
  Synthetic Construct
MPEEVHHGEEEVETFAFQAEIAQLMSLIINTFYSNKEIFLRELISNASDALDKIRYESLTDPSKLDSGKELKIDIIPNPQERTLTLVDTGIGMTKADLINNLGTIAKSGTKAFMEALQFGVGFYSAYLVAEKVVVITKHNDDEQYAWESSAGGSFTVRADHGEPIGRGTKVILHLKEDQTEYLEERRVKEVVKKHSQFIGYPITLYLEKEREKEISDDEAEEEKGEKEEEDKDDEEKPKIEDVGSDEEDDSGKDKKKKTKKIKEKYIDQEELNKTKPIWTRNPDDITQEEYGEFYKSLTNDWEDHLAVKHFSVEGQLEFRALLFIPRRAPFDLFENKKKKNNIKLYVRRVFIMDSCDELIPEYLNFIRGVVDSEDLPLNISREMLQQSKILKVIRKNIVKKCLELFSELAEDKENYKKFYEAFSKNLKLGIHEDSTNRRRLSELLRYHTSQSGDEMTSLSEYVSRMKETQKSIYYITGESKEQVANSAFVERVRKRGFEVVYMTEPIDEYCVQQLKEFDGKSLVSVTKEGLELPEDEEEKKKMEESKAKFENLCKLMKEILDKKVEKVTISNRLVSSPCCIVTSTYGWTANMERIMKAQALRDNSTMGYMMAKKHLEINPDHPIVETLRQKAEADKNDKAVKDLVVLLFETALLSSGFSLEDPQTHSNRIYRMIKLGLGIDEDEVAAEEPNAAVPDEIPPLEGDEDASRMEEVD

SEQ ID NO: 13 - DNA, 2193 bp, Artificial Sequence
  codon-optimized gene of  XP_003501716 (chHSP90 beta)
  CDS (13)..(2187)
aagcttgccaccatgccagaggaggtgcaccatggcgaggaggaggtggagaccttcgcctttcaggctgagatcgcccagctgatgtccctgatcatcaacacattctacagcaataaggagatctttctgcgcgagctgatcagcaatgcttctgacgccctggataagatcagatatgagtctctgaccgacccctctaagctggattccggcaaggagctgaagatcgacatcatcccaaacccccaggagcgcaccctgacactggtggacaccggcatcggcatgacaaaggctgatctgatcaacaatctgggcaccatcgccaagtccggcacaaaggctttcatggaggccctgcaggctggagctgatatctccatgatcggccagttcggcgtgggcttttactccgcctatctggtggccgagaaggtggtggtcatcaccaagcacaatgacgatgagcagtacgcttgggagtccagcgccggaggctccttcacagtgcgcgctgaccacggagagcctatcggaaggggaaccaaagtgatcctgcatctgaaggaggatcagacagagtatctggaggagaggcgggtgaaggaggtggtgaagaagcattcccagtttatcggctaccctatcaccctgtatctggagaaggagagggagaaggagatcagcgacgatgaggccgaggaggagaagggcgagaaggaggaggaggacaaggacgatgaggagaagccaaagatcgaggacgtgggcagcgatgaggaggacgattctggcaaggacaagaagaagaagaccaagaagatcaaggagaagtacatcgatcaggaggagctgaacaagaccaagcctatctggacacgcaatccagacgatatcacccaggaggagtacggcgacttctataagagcctgacaaatgactgggaggatcacctggccgtgaagcatttttctgtggagggccagctggagttcagggctctgctgtttatccccagacgcgcccctttcgatctgtttgagaacaagaagaagaagaacaacatcaagctgtacgtgaggcgggtgttcatcatggacagctgcgatgagctgatcccagagtatctgaacttcatcagaggcgtggtggactctgaggatctgcccctgaacatctcccgggagatgctgcagcagagcaagatcctgaaagtgatcagaaagaatatcgtgaagaagtgtctggagctgttttccgagctggctgaggacaaggagaactacaagaagttctatgaggcctttagcaagaatctgaagctgggcatccacgaggactctaccaacagacgcaggctgtccgagctgctgcggtaccatacctcccagagcggcgatgagatgacatctctgtccgagtacgtgtccagaatgaaggagacccagaagtccatctactatatcacaggcgagagcaaggagcaggtggctaactctgccttcgtggagagggtgccaaagaggggatttgaggccgtgtacatgaccgagcctatcgacgagtattgcgtgcagcagctgaaggagttcgatggcaagtccctggtgagcgtgacaaaggagggactggagctgccagaggacgaggaggagaagaagaagatggaggagagcaaggccaagtttgagaacctgtgcaagctgatgaaggagatcctggataagaaggtggagaaggtgaccatctctaaccggctggtgtcttccccttgctgtatcgtgacctccacatacggctggaccgctaatatggagcggatcatgaaggcccaggctctgagagacaactctacaatgggctatatgatggccaagaagcacctggagatcaatcctgatcatccaatcgtggagacactgagacagaaggccgaggctgacaagaacgataaggctgtgaaggacctggtggtgctgctgttcgagaccgccctgctgagctctggctttagcctggaggatccacagacacactctaaccggatctacagaatgatcaagctgggcctgggcatcgacgaggatgaggtgaccgctgaggagccatctgccgctgtgccagacgagatcccacctctggagggcgacgaggatgcttccaggatggaggaggtggattgagaattc

SEQ ID NO: 14 - protein, 724 aa, Artificial Sequence
  Synthetic Construct
MPEEVHHGEEEVETFAFQAEIAQLMSLIINTFYSNKEIFLRELISNASDALDKIRYESLTDPSKLDSGKELKIDIIPNPQERTLTLVDTGIGMTKADLINNLGTIAKSGTKAFMEALQAGADISMIGQFGVGFYSAYLVAEKVVVITKHNDDEQYAWESSAGGSFTVRADHGEPIGRGTKVILHLKEDQTEYLEERRVKEVVKKHSQFIGYPITLYLEKEREKEISDDEAEEEKGEKEEEDKDDEEKPKIEDVGSDEEDDSGKDKKKKTKKIKEKYIDQEELNKTKPIWTRNPDDITQEEYGDFYKSLTNDWEDHLAVKHFSVEGQLEFRALLFIPRRAPFDLFENKKKKNNIKLYVRRVFIMDSCDELIPEYLNFIRGVVDSEDLPLNISREMLQQSKILKVIRKNIVKKCLELFSELAEDKENYKKFYEAFSKNLKLGIHEDSTNRRRLSELLRYHTSQSGDEMTSLSEYVSRMKETQKSIYYITGESKEQVANSAFVERVPKRGFEAVYMTEPIDEYCVQQLKEFDGKSLVSVTKEGLELPEDEEEKKKMEESKAKFENLCKLMKEILDKKVEKVTISNRLVSSPCCIVTSTYGWTANMERIMKAQALRDNSTMGYMMAKKHLEINPDHPIVETLRQKAEADKNDKAVKDLVVLLFETALLSSGFSLEDPQTHSNRIYRMIKLGLGIDEDEVTAEEPSAAVPDEIPPLEGDEDASRMEEVD

SEQ ID NO: 15 - DNA, 1155 bp, Artificial Sequence
  codon-optimized gene of  NP_008996 (hCDC37)
  CDS (13)..(1149)
aagcttgccaccatggtggactactccgtgtgggatcacatcgaggtgagcgacgatgaggacgagacacatcctaacatcgatacagccagcctgttcagatggagacaccaggctcgcgtggagaggatggagcagtttcagaaggagaaggaggagctggaccggggctgcagagagtgtaagcggaaggtggccgagtgccagagaaagctgaaggagctggaggtggctgagggcggcaaggctgagctggagagactgcaggctgaggctcagcagctgcggaaggaggagagatcctgggagcagaagctggaggagatgaggaagaaggagaagtctatgccctggaacgtggacaccctgtccaaggatggcttctccaagagcatggtgaataccaagcctgagaagacagaggaggatagcgaggaggtgcgcgagcagaagcacaagacattcgtggagaagtatgagaagcagatcaagcattttggcatgctgaggcggtgggacgatagccagaagtacctgtctgacaacgtgcacctggtgtgcgaggagacagctaattacctggtcatctggtgcatcgatctggaggtggaggagaagtgtgccctgatggagcaggtggctcatcagacaatcgtgatgcagttcatcctggagctggccaagtctctgaaggtggacccacgcgcttgttttaggcagttctttaccaagatcaagacagccgaccggcagtacatggagggcttcaacgatgagctggaggcttttaaggagagggtgcggggcagagccaagctgagaatcgagaaggctatgaaggagtacgaggaggaggagcgcaagaagaggctgggacccggcggcctggaccccgtggaggtgtatgagtccctgccagaggagctgcagaagtgctttgacgtgaaggatgtgcagatgctgcaggacgccatcagcaagatggaccctaccgatgctaagtatcatatgcagaggtgtatcgattctggcctgtgggtgccaaattctaaggcctccgaggctaaggagggagaggaggctggacctggcgaccctctgctggaggctgtgcccaagacaggcgacgagaaggatgtgtctgtgtgagaattc

SEQ ID NO: 16 - protein, 378 aa, Artificial Sequence
  Synthetic Construct
MVDYSVWDHIEVSDDEDETHPNIDTASLFRWRHQARVERMEQFQKEKEELDRGCRECKRKVAECQRKLKELEVAEGGKAELERLQAEAQQLRKEERSWEQKLEEMRKKEKSMPWNVDTLSKDGFSKSMVNTKPEKTEEDSEEVREQKHKTFVEKYEKQIKHFGMLRRWDDSQKYLSDNVHLVCEETANYLVIWCIDLEVEEKCALMEQVAHQTIVMQFILELAKSLKVDPRACFRQFFTKIKTADRQYMEGFNDELEAFKERVRGRAKLRIEKAMKEYEEEERKKRLGPGGLDPVEVYESLPEELQKCFDVKDVQMLQDAISKMDPTDAKYHMQRCIDSGLWVPNSKASEAKEGEEAGPGDPLLEAVPKTGDEKDVSV

SEQ ID NO: 17 - DNA, 1158 bp, Artificial Sequence
  codon-optimized gene of XP_003499785 (chCDC37)
  CDS (13)..(1152)
aagcttgccaccatggtggactactccgtgtgggatcacatcgaggtgagcgacgatgaggacgagacccatcctaacatcgatacagccagcctgttcaggtggaggcaccaggctagggtggagaggatggagcagtttcagaaggagaaggacgagctggatcggggctgcagagagtgtaagcggaaggtggccgagtgccagcgcaagctgagggagctggaggtggctgagggaggaggacaggtggagctggagagactgagggctgaggctcagcagctgaggaaggaggagaggtcctgggagcagaagctggaggacatgagaaagaaggagaagtctatgccctggaacgtggacaccctgtccaaggatggcttctccaagagcatcgtgaatatcaagcctgagaaggccgaggaggatagcgaggaggctcgcgagcagaagcataagacattcgtggagaagtatgagaagcagatcaagcactttggcatgctgcatagatgggacgatagccagaagtacctgtctgacaacgtgcacctggtgtgcgaggagaccgccaattacctggtcatctggtgcatcgatctggaggtggaggagaagtgtgccctgatggagcaggtggctcatcagacaatggtcatgcagttcatcctggagctggccaagtctctgaaggtggacccacgggcttgttttagacagttctttaccaagatcaagacagccgatcagcagtacatggagggcttcaagtatgagctggaggcttttaaggagagggtgcggggaagagctaagctgaggatcgagaaggctatgaaggagtacgaggaggaggagcggaagaagagactgggaccaggaggactggacccagtggaggtgtatgagtccctgccagaggagctgcagaagtgcttcgacgtgaaggatgtgcagatgctgcaggacgccatcagcaagatggaccctaccgatgctaagtttcacatgcagaggtgtatcgattctggcctgtgggtgccaaactctaagtccggcgaggccaaggagggagaggaggctggaccaggcgaccctctgctggaggctgtgccaaaggctggcaatgagaaggacgtgtccgcctgagaattc

SEQ ID NO: 18 - protein, 379 aa, Artificial Sequence
  Synthetic Construct
MVDYSVWDHIEVSDDEDETHPNIDTASLFRWRHQARVERMEQFQKEKDELDRGCRECKRKVAECQRKLRELEVAEGGGQVELERLRAEAQQLRKEERSWEQKLEDMRKKEKSMPWNVDTLSKDGFSKSIVNIKPEKAEEDSEEAREQKHKTFVEKYEKQIKHFGMLHRWDDSQKYLSDNVHLVCEETANYLVIWCIDLEVEEKCALMEQVAHQTMVMQFILELAKSLKVDPRACFRQFFTKIKTADQQYMEGFKYELEAFKERVRGRAKLRIEKAMKEYEEEERKKRLGPGGLDPVEVYESLPEELQKCFDVKDVQMLQDAISKMDPTDAKFHMQRCIDSGLWVPNSKSGEAKEGEEAGPGDPLLEAVPKAGNEKDVSA

SEQ ID NO: 19 - DNA, 1740 bp, Artificial Sequence
  codon-optimized gene of NP_955472 (hHSP60)
  CDS (13)..(1734)
aagcttgccaccatgctgagactgcccacagtgttcagacagatgcgccctgtgagcagggtgctggccccacacctgacccgggcctacgctaaggacgtgaagtttggcgccgatgctagagccctgatgctgcagggagtggacctgctggctgatgctgtggctgtgacaatgggccctaagggccgcaccgtgatcatcgagcagtcttggggctccccaaaggtgacaaaggacggcgtgaccgtggccaagtctatcgacctgaaggataagtataagaacatcggcgccaagctggtgcaggacgtggctaacaatacaaatgaggaggccggcgatggcaccacaacagctaccgtgctggctagatctatcgctaaggagggcttcgagaagatctccaagggcgctaaccctgtggagattagacggggcgtgatgctggctgtggatgctgtgatcgccgagctgaagaagcagtctaagcccgtgacaacccctgaggagatcgctcaggtggccaccatcagcgccaacggcgacaaggagatcggcaatatcatctctgatgctatgaagaaggtgggcagaaagggcgtgatcacagtgaaggacggcaagaccctgaacgatgagctggagatcatcgagggcatgaagttcgaccggggctacatcagcccatattttatcaatacatctaagggccagaagtgcgagtttcaggatgcctacgtgctgctgtccgagaagaagatctccagcatccagagcatcgtgccagctctggagatcgccaacgctcacaggaagcccctggtcatcatcgctgaggacgtggatggcgaggccctgtccaccctggtgctgaataggctgaaggtcggcctgcaggtggtggctgtgaaggctcctggatttggcgacaacagaaagaatcagctgaaggatatggccatcgctacaggcggcgccgtgtttggagaggagggcctgaccctgaacctggaggacgtgcagcctcatgatctgggcaaggtcggcgaagtgatcgtgaccaaggacgatgccatgctgctgaagggcaagggcgacaaggctcagatcgagaagagaatccaggagatcatcgagcagctggatgtgacaaccagcgagtacgagaaggagaagctgaatgagagactggctaagctgtctgatggagtggccgtgctgaaggtgggcggcacaagcgatgtggaggtgaacgagaagaaggacagagtgacagatgctctgaatgccacccgcgccgctgtggaggagggaatcgtgctgggcggcggctgtgctctgctgagatgtatccccgccctggactctctgacccctgctaacgaggatcagaagatcggcatcgagatcatcaagcgcacactgaagatcccagccatgaccatcgctaagaatgctggagtggagggctccctgattgtggagaagatcatgcagtcttccagcgaagtgggctatgacgccatggctggcgatttcgtgaatatggtggagaagggcatcatcgaccccacaaaggtggtgaggacagctctgctggatgctgctggagtggcctccctgctgacaaccgctgaggtggtggtgaccgagatcccaaaggaggagaaggaccccggcatgggcgctatgggcggcatgggcggcggcatgggcggcggcatgttttgagaattc

SEQ ID NO: 20 - protein, 573 aa, Artificial Sequence
  Synthetic Construct
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 21 - DNA, 1212 bp, Artificial Sequence
  codon-optimized gene of NP_001530 (hHSP40)
  CDS (13)..(1206)
aagcttgccaccatggtgaaggagacaacatactatgacgtgctgggcgtgaagccaaacgccacccaggaggagctgaagaaggcctaccggaagctggctctgaagtatcaccccgataagaaccctaatgagggcgagaagttcaagcagatctctcaggcctacgaggtgctgtccgacgctaagaagagagagctgtatgataagggcggcgagcaggccatcaaggagggcggcgccggcggcggctttggctctcccatggacatctttgatatgttctttgggggcgggggaagaatgcagagagagaggcggggcaagaatgtggtgcatcagctgtccgtgaccctggaggacctgtacaacggcgccacacggaagctggctctgcagaagaatgtgatctgcgataagtgtgagggcagaggcggcaagaagggcgctgtggagtgctgtcctaactgccgcggcaccggcatgcagatcagaatccaccagatcggcccaggcatggtgcagcagatccagagcgtgtgcatggagtgtcagggccatggcgagcggatcagccctaaggacagatgcaagtcttgtaatggccgcaagatcgtgagggagaagaagatcctggaggtgcacatcgacaagggcatgaaggatggccagaagatcacattccatggcgagggcgaccaggagccaggcctggagccaggcgatatcatcatcgtgctggaccagaaggatcacgccgtgttcaccagacgcggcgaggacctgtttatgtgcatggatatccagctggtggaggctctgtgtggctttcagaagccaatctccacactggacaaccgcaccatcgtgatcacaagccaccccggccagatcgtgaagcatggcgatatcaagtgcgtgctgaatgagggcatgcctatctacaggcggccatatgagaagggccgcctgatcatcgagttcaaggtgaactttcccgagaatggcttcctgtcccctgataagctgagcctgctggagaagctgctgccagagaggaaggaggtggaggagacagacgagatggatcaggtggagctggtggactttgatcccaaccaggagagacgcaggcattacaatggcgaggcctatgaggacgatgagcaccatcctaggggcggcgtgcagtgtcagacatcttgagaattc

SEQ ID NO: 22 - protein, 397 aa, Artificial Sequence
  Synthetic Construct
MVKETTYYDVLGVKPNATQEELKKAYRKLALKYHPDKNPNEGEKFKQISQAYEVLSDAKKRELYDKGGEQAIKEGGAGGGFGSPMDIFDMFFGGGGRMQRERRGKNVVHQLSVTLEDLYNGATRKLALQKNVICDKCEGRGGKKGAVECCPNCRGTGMQIRIHQIGPGMVQQIQSVCMECQGHGERISPKDRCKSCNGRKIVREKKILEVHIDKGMKDGQKITFHGEGDQEPGLEPGDIIIVLDQKDHAVFTRRGEDLFMCMDIQLVEALCGFQKPISTLDNRTIVITSHPGQIVKHGDIKCVLNEGMPIYRRPYEKGRLIIEFKVNFPENGFLSPDKLSLLEKLLPERKEVEETDEMDQVELVDFDPNQERRRHYNGEAYEDDEHHPRGGVQCQTS

SEQ ID NO: 23 - DNA, 327 bp, Artificial Sequence
  codon-optimized gene of NP_002148 (hHSP10)
  CDS (13)..(321)
aagcttgccaccatggccggccaggctttcagaaagtttctgccactgtttgacagggtgctggtggagagatctgctgctgagacagtgacaaagggcggcatcatgctgcccgagaagtcccagggcaaggtgctgcaggctacagtggtggctgtgggctccggcagcaagggcaagggcggcgagatccagcccgtgtccgtgaaagtgggcgataaggtgctgctgcctgagtacggcggcacaaaggtggtgctggacgataaggactatttcctgtttcgcgacggcgatatcctgggcaagtacgtggattgagaattc

SEQ ID NO: 24 - protein, 102 aa, Artificial Sequence
  Synthetic Construct
MAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 25 - DNA, 2595 bp, Artificial Sequence
  codon-optimized gene of NP_006635 (hHSP110)
  CDS (13)..(2589)
aagcttgccaccatgtccgtggtgggcctggatgtgggcagccagtcttgttacattgctgtggctagagccggcggcattgagacaatcgccaacgagttcagcgacaggtgcaccccatctgtgatctcctttggcagcaagaatcggaccatcggcgtggccgctaagaaccagcagatcacccacgctaacaatacagtgtctaatttcaagaggtttcatggccgggccttcaacgatccctttatccagaaggagaaggagaatctgtcctacgacctggtgcctctgaagaacggcggcgtgggcatcaaagtgatgtatatgggcgaggagcatctgttctccgtcgagcagatcaccgctatgctgctgacaaagctgaaggagacagccgagaatagcctgaagaagcccgtgacagactgtgtgatctctgtgccttccttctttaccgacgctgagaggcggtctgtgctggatgccgctcagatcgtgggcctgaattgcctgagactgatgaacgatatgacagctgtggccctgaactacggcatctataagcaggacctgccttccctggatgagaagccacgcatcgtggtgttcgtggacatgggccactccgctttccaggtgagcgcttgtgcctttaataagggcaagctgaaggtgctgggcacagccttcgatccatttctgggcggcaagaactttgacgagaagctggtggagcatttctgcgccgagtttaagaccaagtacaagctggatgctaagagcaagatcagagccctgctgcgcctgtatcaggagtgcgagaagctgaagaagctgatgtccagcaattctaccgacctgcccctgaatatcgagtgtttcatgaacgacaaggatgtgagcggcaagatgaaccgctctcagtttgaggagctgtgcgctgagctgctgcagaagatcgaggtgcctctgtatagcctgctggagcagacacacctgaaggtggaggatgtgtctgccgtggagatcgtgggcggcgctaccagaatcccagccgtgaaggagcgcatcgctaagttctttggcaaggacatctccaccacactgaacgctgatgaggctgtggccaggggctgtgctctgcagtgtgctatcctgtctcccgctttcaaggtgcgggagttttccgtgacagacgccgtgccattccccatctccctgatctggaatcatgacagcgaggataccgagggcgtgcacgaggtgttctccaggaaccatgccgctcctttctccaaggtgctgacctttctgagacgcggcccattcgagctggaggccttttacagcgatccccagggcgtgccttatccagaggctaagatcggccgcttcgtggtgcagaatgtgagcgcccagaaggacggcgagaagtctagggtgaaggtgaaggtgcgggtgaacacacacggcatctttaccatcagcacagcttctatggtggagaaggtgcccaccgaggagaacgagatgtcttccgaggccgacatggagtgtctgaatcagaggccccctgagaacccagacacagataagaatgtgcagcaggataactctgaggctggcacccagccacaggtgcagacagatgctcagcagacctcccagagcccaccctctcctgagctgacatccgaggagaataagatcccagacgctgataaggccaacgagaagaaggtggatcagcctccagaggctaagaagcccaagatcaaggtggtgaatgtggagctgcctatcgaggccaacctggtgtggcagctgggcaaggacctgctgaatatgtacatcgagacagagggcaagatgatcatgcaggacaagctggagaaggagagaaatgatgctaagaacgccgtggaggagtacgtgtatgagttccgcgataagctgtgcggcccttatgagaagttcatctgtgagcaggaccatcagaactttctgcggctgctgaccgagacagaggactggctgtacgaggagggcgaggatcaggctaagcaggcctatgtggacaagctggaggagctgatgaagatcggcaccccagtgaaggtgaggttccaggaggctgaggagcggcccaagatgtttgaggagctgggccagagactgcagcactacgccaagatcgccgctgactttcgcaataaggatgagaagtataaccatatcgatgagtccgagatgaagaaggtggagaagagcgtgaatgaagtgatggagtggatgaacaatgtgatgaacgctcaggccaagaagagcctggaccaggaccccgtggtgagggctcaggagatcaagacaaagatcaaggagctgaacaatacatgtgagcctgtggtgacccagcccaagcctaagatcgagtctccaaagctggagcggaccccaaacggccccaatatcgacaagaaggaggaggacctggaggataagaacaattttggagctgagccccctcaccagaatggagagtgctacccaaacgagaagaactccgtgaacatggacctggattgagaattc

SEQ ID NO: 26 - protein, 858 aa, Artificial Sequence
  Synthetic Construct
MSVVGLDVGSQSCYIAVARAGGIETIANEFSDRCTPSVISFGSKNRTIGVAAKNQQITHANNTVSNFKRFHGRAFNDPFIQKEKENLSYDLVPLKNGGVGIKVMYMGEEHLFSVEQITAMLLTKLKETAENSLKKPVTDCVISVPSFFTDAERRSVLDAAQIVGLNCLRLMNDMTAVALNYGIYKQDLPSLDEKPRIVVFVDMGHSAFQVSACAFNKGKLKVLGTAFDPFLGGKNFDEKLVEHFCAEFKTKYKLDAKSKIRALLRLYQECEKLKKLMSSNSTDLPLNIECFMNDKDVSGKMNRSQFEELCAELLQKIEVPLYSLLEQTHLKVEDVSAVEIVGGATRIPAVKERIAKFFGKDISTTLNADEAVARGCALQCAILSPAFKVREFSVTDAVPFPISLIWNHDSEDTEGVHEVFSRNHAAPFSKVLTFLRRGPFELEAFYSDPQGVPYPEAKIGRFVVQNVSAQKDGEKSRVKVKVRVNTHGIFTISTASMVEKVPTEENEMSSEADMECLNQRPPENPDTDKNVQQDNSEAGTQPQVQTDAQQTSQSPPSPELTSEENKIPDADKANEKKVDQPPEAKKPKIKVVNVELPIEANLVWQLGKDLLNMYIETEGKMIMQDKLEKERNDAKNAVEEYVYEFRDKLCGPYEKFICEQDHQNFLRLLTETEDWLYEEGEDQAKQAYVDKLEELMKIGTPVKVRFQEAEERPKMFEELGQRLQHYAKIAADFRNKDEKYNHIDESEMKKVEKSVNEVMEWMNNVMNAQAKKSLDQDPVVRAQEIKTKIKELNNTCEPVVTQPKPKIESPKLERTPNGPNIDKKEEDLEDKNNFGAEPPHQNGECYPNEKNSVNMDLD

SEQ ID NO: 27 - DNA, 1767 bp, Artificial Sequence
  codon-optimized gene of chHSP70
  CDS (13)..(1761)
aagcttgccaccatggctaagaacaccgccatcggcatcgacctgggcaccacatactcctgcgtgggcgtgttccagcacggcaaggtggagatcatcgccaacgatcagggcaatcggaccacacctagctatgtggcttttaccgacacagagagactgatcggcgatgccgctaagaatcaggtggccctgaacccacagaataccgtgttcgacgctaagagactgatcggccgcaagtttggcgacgccgtggtgcagagcgatatgaagcactggccattccaggtggtgaacgacggcgataagcccaaggtgcaagtgaattacaagggcgagtccaggtccttcttccccgaggagatctccagcatggtgctgacaaagatgaaggagatcgctgaggcctacctgggccatcccgtgaccaacgccgtgatcacagtgcctgcttacttcaacgactctcagcggcaggctaccaaggatgccggcgtgatcgctggcctgaacgtgctgagaatcatcaatgagcctacagccgctgccatcgcctacggcctggacagaaaccgccagggcaagaaggatatctctcagaataagctggctgtgaggcggctgaggaccgcttgcgagagggctaagcggaccctgtcttccagcacacaggccagcctggagatcgactctctgttcgagggcatcgacttctacacctccatcacaagggctcggttcgaggagctgtgctccgacctgtttaggagcaccctggagccagtggagaaggccctgagggacgctaagatggataaggcccagatccatgatctggtgctggtgggaggcagcacacgcatcccaaaggtgcagaagctgctgcaggacttctttaacggcagggatctgaacaagtctatcaatccagacgaggctgtggcttatggagctgctgtgcaggctgctatcctgatgggcgataagtccgagaatgtgcaggacctgctgctgctggatgtggctcctctgagcctgggcctggagaccgctggaggcgtgatgacagctctgatcaagcgcaacagcaccatccccacaaagcagacccagacattcaccacatactctgacaatcagcctggcgtgctgatccaggtgtatgagggcgagagagccatgaccggcgataacaatctgctgggccgcttcgagctgtccggcatcccccctgctccaaggggcgtgccccagatcgaggtgacctttgacatcgatgccaacggcctgaatgtgaccgctacagacaagtctacaggcaaggccaacaagatcaccatcacaaatgataagggcaggctgtccaaggaggagatcgagagaatggtgcaggaggccgagaggtacaaggctgaggacgaggtgcagagagatcgcgtggctgccaagaacgccctggagtcttatgcttttaatatgaagtccgccgtggagggagagggcctgaagggcaagctgtccgaggctgacaagaagaaggtgctggacaagtgccaggaagtgatctcctggctggacagcaacaccctggccgataaggaggagttcgtgcacaagagggaggagctggagagggtgtgctctcctatcatctccggcctgtaccagggagctggagctccaggagctggaggctttggagctcaggctcctaagggagctagcggctctggaccaacaatcgaggaggtggactgagaattc

SEQ ID NO: 28 - protein, 582 aa, Artificial Sequence
  Synthetic Construct
MAKNTAIGIDLGTTYSCVGVFQHGKVEIIANDQGNRTTPSYVAFTDTERLIGDAAKNQVALNPQNTVFDAKRLIGRKFGDAVVQSDMKHWPFQVVNDGDKPKVQVNYKGESRSFFPEEISSMVLTKMKEIAEAYLGHPVTNAVITVPAYFNDSQRQATKDAGVIAGLNVLRIINEPTAAAIAYGLDRNRQGKKDISQNKLAVRRLRTACERAKRTLSSSTQASLEIDSLFEGIDFYTSITRARFEELCSDLFRSTLEPVEKALRDAKMDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQTFTTYSDNQPGVLIQVYEGERAMTGDNNLLGRFELSGIPPAPRGVPQIEVTFDIDANGLNVTATDKSTGKANKITITNDKGRLSKEEIERMVQEAERYKAEDEVQRDRVAAKNALESYAFNMKSAVEGEGLKGKLSEADKKKVLDKCQEVISWLDSNTLADKEEFVHKREELERVCSPIISGLYQGAGAPGAGGFGAQAPKGASGSGPTIEEVD

SEQ ID NO: 29 - DNA, 630 bp, Artificial Sequence
  codon-optimized gene of chHSP27
  CDS (13)..(624)
aagcttgccaccatgaccgagaggcgggtgcctttctccctgctgaggtctccatcctgggagccctttcgggactggtacccagctcacagcagactgttcgatcaggcctttggcgtgccaaggctgcctgacgagtggagccagtggttctctgccgctggatggccaggatatgtgaggccactgccagctgctaccgctgagggacctgctgctgtgacactggctgctccagccttttccagggctctgaacaggcagctgtccagcggcgtgagcgagatcaggcagaccgctgacagatggcgcgtgtctctggatgtgaatcatttcgcccctgaggagctgaccgtgaagacaaaggagggcgtggtggagatcacaggcaagcacgaggagagacaggatgagcatggctacatctccagatgctttacccgcaagtatacactgccacctggagtggaccccaccctggtgtcttccagcctgtctcctgagggcaccctgacagtggaggctcctctgccaaaggctgtgacacagtccgccgagatcaccatcccagtgacattcgaggcccgcgctcagatcggcggccccgaggccggcaaggtgtgagaattc

SEQ ID NO: 30 - protein, 203 aa, Artificial Sequence
  Synthetic Construct
MTERRVPFSLLRSPSWEPFRDWYPAHSRLFDQAFGVPRLPDEWSQWFSAAGWPGYVRPLPAATAEGPAAVTLAAPAFSRALNRQLSSGVSEIRQTADRWRVSLDVNHFAPEELTVKTKEGVVEITGKHEERQDEHGYISRCFTRKYTLPPGVDPTLVSSSLSPEGTLTVEAPLPKAVTQSAEITIPVTFEARAQIGGPEAGKV

SEQ ID NO: 31 - DNA, 744 bp, Artificial Sequence
  codon-optimized gene of NP_002497 (NGF)
  CDS (13)..(738)
aagcttgccaccatgtctatgctgttctacacactgatcaccgcttttctgatcggcatccaggccgagcctcattccgagagcaacgtgcctgctggccacacaatcccacaggcccattggaccaagctgcagcactctctggacacagctctgaggagggctcgctccgccccagctgctgccatcgctgcccgggtggccggccagacaagaaatatcaccgtggacccccggctgttcaagaagagacgcctgagatcccctcgcgtgctgtttagcacacagccccctagagaggctgccgatacccaggacctggatttcgaagtgggaggagctgctcccttcaacaggacccaccggagcaagagatccagctctcaccccatcttccataggggcgagttctccgtgtgcgactccgtgtccgtgtgggtgggcgacaagaccacagctacagatatcaagggcaaggaagtgatggtgctgggcgaagtgaatatcaacaattccgtgttcaagcagtacttctttgagaccaagtgccgcgacccaaaccccgtggatagcggctgtaggggcatcgatagcaagcattggaactcctattgtaccacaacccacacattcgtgaaggctctgaccatggacggcaagcaggctgcctggcgctttatcaggatcgataccgcctgcgtgtgcgtgctgtccaggaaggctgtgaggcgggcctgagaattc

SEQ ID NO: 32 - protein, 241 aa, Artificial Sequence
  Synthetic Construct
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 33 - DNA, 744 bp, Artificial Sequence
  codon-optimized gene of NP_002599 (PDGF beta)
  CDS (13)..(738)
aagcttgccaccatgaacaggtgctgggccctgttcctgtccctgtgctgttacctgaggctggtgtccgccgagggcgatcctatcccagaggagctgtatgagatgctgtctgaccactccatcaggtccttcgacgatctgcagcgcctgctgcatggcgaccctggagaggaggatggagctgagctggacctgaatatgaccagatcccacagcggaggagagctggagagcctggctaggggcaggcggtctctgggctccctgacaatcgctgagccagccatgatcgctgagtgcaagaccagaacagaggtgttcgagatctctagacgcctgatcgatcgcaccaacgccaattttctggtgtggcccccttgcgtggaggtgcagaggtgttccggctgctgtaacaatagaaacgtgcagtgtcgcccaacacaggtgcagctgaggccagtgcaggtgcggaagatcgagatcgtgcggaagaagcccatcttcaagaaggccaccgtgacactggaggatcacctggcttgcaagtgtgagaccgtggctgctgctaggccagtgacacggagccctggaggatctcaggagcagagggctaagacccctcagacacgggtgaccatcagaacagtgcgcgtgaggcggccaccaaagggcaagcataggaagtttaagcacacccatgacaagacagccctgaaggagaccctgggcgcttgagaattc

SEQ ID NO: 34 - protein, 241 aa, Artificial Sequence
  Synthetic Construct
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 35 - DNA, 510 bp, Artificial Sequence
  codon-optimized gene of NP_443104 (IL-17F)
  CDS (13)..(504)
aagcttgccaccatgaccgtgaagacactgcacggccccgctatggtgaagtacctgctgctgtccatcctgggactggccttcctgagcgaggctgctgctaggaagatccctaaagtgggccataccttctttcagaagcctgagtcttgcccacctgtgccaggaggatccatgaagctggacatcggcatcatcaacgagaatcagagagtgtccatgagccgcaacatcgagtctaggtccacaagcccctggaactacaccgtgacatgggacccaaatcggtatcccagcgaggtggtgcaggctcagtgcagaaacctgggctgtatcaatgcccagggcaaggaggatatcagcatgaactccgtgccaatccagcaggagaccctggtggtgaggcggaagcaccagggctgttccgtgtccttccagctggagaaggtgctggtgacagtgggctgcacctgcgtgacacccgtgatccaccatgtgcagtgagaattc

SEQ ID NO: 36 - protein, 163 aa, Artificial Sequence
  Synthetic Construct
MTVKTLHGPAMVKYLLLSILGLAFLSEAAARKIPKVGHTFFQKPESCPPVPGGSMKLDIGIINENQRVSMSRNIESRSTSPWNYTVTWDPNRYPSEVVQAQCRNLGCINAQGKEDISMNSVPIQQETLVVRRKHQGCSVSFQLEKVLVTVGCTCVTPVIHHVQ

SEQ ID NO: 37 - DNA, 654 bp, Artificial Sequence
  codon-optimized gene of NP_000505 (GDNF)
  CDS (13)..(648)
aagcttgccaccatgaagctgtgggatgtggtggccgtgtgcctggtgctgctgcacaccgcttccgctttcccactgccagctggcaagaggccacctgaggctccagctgaggatagatccctgggcaggcggagagctccctttgctctgtccagcgacagcaacatgcctgaggattacccagaccagttcgacgatgtgatggacttcatccaggccacaatcaagcgcctgaagaggagccccgacaagcagatggctgtgctgcctcgcagggagcggaatagacaggccgctgccgctaaccctgagaactccaggggcaagggccggagaggacagaggggcaagaacagaggctgcgtgctgaccgccatccatctgaatgtgacagatctgggcctgggctacgagaccaaggaggagctgatcttccggtattgctctggctcctgtgatgccgctgagaccacatacgacaagatcctgaagaacctgagccgcaatcgcaggctggtgtctgacaaagtgggccaggcctgctgtaggccaatcgctttcgacgatgacctgtcttttctggatgacaacctggtgtatcacatcctgcgcaagcattccgctaagaggtgcggctgcatctgagaattc

SEQ ID NO: 38 - protein, 211 aa, Artificial Sequence
  Synthetic Construct
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 39 - DNA, 792 bp, Artificial Sequence
  codon-optimized gene of NP_002518 (NT3)
  CDS (13)..(786)
aagcttgccaccatgtctatcctgttctacgtgatctttctggcctatctgaggggcatccagggcaacaatatggaccagcggtccctgccagaggattccctgaacagcctgatcatcaagctgatccaggctgacatcctgaagaacaagctgagcaagcagatggtggatgtgaaggagaattaccagtctaccctgccaaaggctgaggctccaagagagcctgagaggggaggacctgctaagtccgctttccagcccgtgatcgccatggacaccgagctgctgagacagcagaggcggtataacagccctcgcgtgctgctgtctgactccacaccactggagccccctccactgtacctgatggaggattatgtgggctctcctgtggtggccaataggacctccagacgcaagcggtacgctgagcacaagagccataggggcgagtattccgtgtgcgatagcgagtctctgtgggtgacagacaagtccagcgccatcgatatcaggggacaccaggtgaccgtgctgggagagatcaagacaggcaatagccccgtgaagcagtacttttatgagaccagatgcaaggaggctcgccctgtgaagaacggctgtagaggcatcgacgataagcattggaactcccagtgtaagacctcccagacatacgtgcgcgccctgacatctgagaacaataagctggtgggctggaggtggatccggatcgacacctcctgcgtgtgcgctctgagcaggaagatcggccggacatgagaattc

SEQ ID NO: 40 - protein, 257 aa, Artificial Sequence
  Synthetic Construct
MSILFYVIFLAYLRGIQGNNMDQRSLPEDSLNSLIIKLIQADILKNKLSKQMVDVKENYQSTLPKAEAPREPERGGPAKSAFQPVIAMDTELLRQQRRYNSPRVLLSDSTPLEPPPLYLMEDYVGSPVVANRTSRRKRYAEHKSHRGEYSVCDSESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSRKIGRT

SEQ ID NO: 41 - DNA, 519 bp, Artificial Sequence
  codon-optimized gene of NP_000610 (IFN gamma)
  CDS (13)..(513)
aagcttgccaccatgaagtacacctcctatatcctggccttccagctgtgcatcgtgctgggcagcctgggctgctactgtcaggacccctatgtgaaggaggctgagaacctgaagaagtacttcaatgccggccacagcgacgtggctgataacggcaccctgtttctgggcatcctgaagaattggaaggaggagtctgatagaaagatcatgcagtcccagatcgtgtccttctacttcaagctgttcaagaactttaaggacgatcagtctatccagaagtccgtggagacaatcaaggaggacatgaatgtgaagttctttaactctaacaagaagaagagggacgatttcgagaagctgaccaactactccgtgacagatctgaatgtgcagcggaaggccatccatgagctgatccaggtcatggctgagctgtcccctgccgctaagacaggcaagagaaagcgcagccagatgctgtttcgcggcaggcgggcctctcagtgagaattc

SEQ ID NO: 42 - protein, 166 aa, Artificial Sequence
  Synthetic Construct
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 43 - DNA, 762 bp, Artificial Sequence
  codon-optimized gene of NP_733931.1 (BDNF)
  CDS (13)..(756)
aagcttgccaccatgaccatcctgttcctgacaatggtcatctcttactttggctgcatgaaggccgctccaatgaaggaggctaacatcaggggacagggaggactggcttatccaggcgtgcggacccacggcacactggagtccgtgaatggaccaaaggctggaagcaggggactgacctctctggctgacacattcgagcatgtgatcgaggagctgctggacgaggatcagaaggtgagacccaacgaggagaacaataaggacgccgatctgtacaccagccgcgtgatgctgtccagccaggtgcctctggagccacctctgctgtttctgctggaggagtacaagaactatctggatgccgctaatatgtccatgagagtgaggcggcacagcgacccagctagaaggggagagctgtccgtgtgcgattctatctccgagtgggtgaccgccgctgacaagaagacagctgtggatatgagcggcggcaccgtgacagtgctggagaaggtgcccgtgtccaagggccagctgaagcagtacttctatgagaccaagtgcaacccaatgggctacacaaaggagggctgtaggggcatcgacaagcggcattggaactcccagtgtagaaccacacagtcctatgtgcgcgctctgaccatggactccaagaagaggatcggctggaggttcatccgcatcgatacaagctgcgtgtgcaccctgacaatcaagaggggccggtgagaattc

SEQ ID NO: 44 - protein, 247 aa, Artificial Sequence
  Synthetic Construct
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 45 - DNA, 3887 bp, Homo sapiens
  CDS (346)..(2910): NM_001017963 WT, CDS 346-2910
gactgcgcaggcgtgctcacctggcgtgctccacccgactgggcgtccgcaggctcctcccccgggtgtggcctccgggcggcatggctgcttcccaggtgatgccggcttcagctagtggggtctagttgaccgttccgcagccgccagggccagcggaaagccggtcagggggaaccgcggcggggctggtgtcatgagcctgaggtgaacttgagggtgcctcctcagcggtctcccgccctgccctgaggggcgccgggaccccaaagagcggaggaagagcgccaccccgacggccaccgcttcggagccagcacgcggggtaccctacggggagcgcggatgcccccgtgttcgggcggggacggctccacccctcctgggccctcccttcgggacagggactgtcccgcccagagtgctgaatacccgcgcgaccgtctggatccccgcccaggaagcccctctgaagcctcctcgccgccgtttctgagaagcagggcacctgttaactggtaccaagaaaaggcccaagtgtttctctggcatctgatggtgtctggatccaccactctactctgtctctggaaacagcccttccacgtctctgcattccctgtcaccgcgtcactggccttcagacagagccaaggtgcagggcaacacctctacaaggatctgcagccatttatattgcttaggctactgatgcctgaggaaacccagacccaagaccaaccgatggaggaggaggaggttgagacgttcgcctttcaggcagaaattgcccagttgatgtcattgatcatcaatactttctactcgaacaaagagatctttctgagagagctcatttcaaattcatcagatgcattggacaaaatccggtatgaaagcttgacagatcccagtaaattagactctgggaaagagctgcatattaaccttataccgaacaaacaagatcgaactctcactattgtggatactggaattggaatgaccaaggctgacttgatcaataaccttggtactatcgccaagtctgggaccaaagcgttcatggaagctttgcaggctggtgcagatatctctatgattggccagttcggtgttggtttttattctgcttatttggttgctgagaaagtaactgtgatcaccaaacataacgatgatgagcagtacgcttgggagtcctcagcagggggatcattcacagtgaggacagacacaggtgaacctatgggtcgtggaacaaaagttatcctacacctgaaagaagaccaaactgagtacttggaggaacgaagaataaaggagattgtgaagaaacattctcagtttattggatatcccattactctttttgtggagaaggaacgtgataaagaagtaagcgatgatgaggctgaagaaaaggaagacaaagaagaagaaaaagaaaaagaagagaaagagtcggaagacaaacctgaaattgaagatgttggttctgatgaggaagaagaaaagaaggatggtgacaagaagaagaagaagaagattaaggaaaagtacatcgatcaagaagagctcaacaaaacaaagcccatctggaccagaaatcccgacgatattactaatgaggagtacggagaattctataagagcttgaccaatgactgggaagatcacttggcagtgaagcatttttcagttgaaggacagttggaattcagagcccttctatttgtcccacgacgtgctccttttgatctgtttgaaaacagaaagaaaaagaacaacatcaaattgtatgtacgcagagttttcatcatggataactgtgaggagctaatccctgaatatctgaacttcattagaggggtggtagactcggaggatctccctctaaacatatcccgtgagatgttgcaacaaagcaaaattttgaaagttatcaggaagaatttggtcaaaaaatgcttagaactctttactgaactggcggaagataaagagaactacaagaaattctatgagcagttctctaaaaacataaagcttggaatacacgaagactctcaaaatcggaagaagctttcagagctgttaaggtactacacatctgcctctggtgatgagatggtttctctcaaggactactgcaccagaatgaaggagaaccagaaacatatctattatatcacaggtgagaccaaggaccaggtagctaactcagcctttgtggaacgtcttcggaaacatggcttagaagtgatctatatgattgagcccattgatgagtactgtgtccaacagctgaaggaatttgaggggaagactttagtgtcagtcaccaaagaaggcctggaacttccagaggatgaagaagagaaaaagaagcaggaagagaaaaaaacaaagtttgagaacctctgcaaaatcatgaaagacatattggagaaaaaagttgaaaaggtggttgtgtcaaaccgattggtgacatctccatgctgtattgtcacaagcacatatggctggacagcaaacatggagagaatcatgaaagctcaagccctaagagacaactcaacaatgggttacatggcagcaaagaaacacctggagataaaccctgaccattccattattgagaccttaaggcaaaaggcagaggctgataagaacgacaagtctgtgaaggatctggtcatcttgctttatgaaactgcgctcctgtcttctggcttcagtctggaagatccccagacacatgctaacaggatctacaggatgatcaaacttggtctgggtattgatgaagatgaccctactgctgatgataccagtgctgctgtaactgaagaaatgccaccccttgaaggagatgacgacacatcacgcatggaagaagtagactaatctctggctgagggatgacttacctgttcagtactctacaattcctctgataatatattttcaaggatgtttttctttatttttgttaatattaaaaagtctgtatggcatgacaactactttaaggggaagataagatttctgtctactaagtgatgctgtgataccttaggcactaaagcagagctagtaatgctttttgagtttcatgttggtttattttcacagattggggtaacgtgcactgtaagacgtatgtaacatgatgttaactttgtggtctaaagtgtttagctgtcaagccggatgcctaagtagaccaaatcttgttattgaagtgttctgagctgtatcttgatgtttagaaaagtattcgttacatcttgtaggatctactttttgaacttttcattccctgtagttgacaattctgcatgtactagtcctctagaaataggttaaactgaagcaacttgatggaaggatctctccacagggcttgttttccaaagaaaagtattgtttggaggagcaaagttaaaagcctacctaagcatatcgtaaagctgttcaaaaataactcagacccagtcttgtggatggaaatgtagtgctcgagtcacattctgcttaaagttgtaacaaatacagatgagttaaaagatattgtgtgacagtgtcttatttagggggaaaggggagtatctggatgacagttagtgccaaaatgtaaaacatgaggcgctagcaggagatggttaaacactagctgctccaagggttgacatggtcttcccagcatgtactcagcaggtgtggggtggagcacacgtaggcacagaaaacaggaatgcagacaacatgcatcccctgcgtccatgagttacatgtgttctcttagtgtccacgttgttttgatgttattcatggaataccttctgtgttaaatacagtcacttaattccttggccttaaaa

SEQ ID NO: 46 - protein, 854 aa, Homo sapiens; identical to SEQ ID NO: 2

SEQ ID NO: 47 - DNA, 3366 bp, Homo sapiens
  CDS (191)..(2389): NM_005348 WT, CDS 191-2389
gcatgcgtaggcgcgcggccgcggcggcggctggggagggttcttccggaaggttcgggaggcttctggaaaaagcgccgcgcgctgggcgggcccgtcgctatataaggcaggcgcgggggtggcgcgtcagttgcttcagcgtcccggtgtggctgtgccgttggtcctgtgcggtcacttagccaagatgcctgaggaaacccagacccaagaccaaccgatggaggaggaggaggttgagacgttcgcctttcaggcagaaattgcccagttgatgtcattgatcatcaatactttctactcgaacaaagagatctttctgagagagctcatttcaaattcatcagatgcattggacaaaatccggtatgaaagcttgacagatcccagtaaattagactctgggaaagagctgcatattaaccttataccgaacaaacaagatcgaactctcactattgtggatactggaattggaatgaccaaggctgacttgatcaataaccttggtactatcgccaagtctgggaccaaagcgttcatggaagctttgcaggctggtgcagatatctctatgattggccagttcggtgttggtttttattctgcttatttggttgctgagaaagtaactgtgatcaccaaacataacgatgatgagcagtacgcttgggagtcctcagcagggggatcattcacagtgaggacagacacaggtgaacctatgggtcgtggaacaaaagttatcctacacctgaaagaagaccaaactgagtacttggaggaacgaagaataaaggagattgtgaagaaacattctcagtttattggatatcccattactctttttgtggagaaggaacgtgataaagaagtaagcgatgatgaggctgaagaaaaggaagacaaagaagaagaaaaagaaaaagaagagaaagagtcggaagacaaacctgaaattgaagatgttggttctgatgaggaagaagaaaagaaggatggtgacaagaagaagaagaagaagattaaggaaaagtacatcgatcaagaagagctcaacaaaacaaagcccatctggaccagaaatcccgacgatattactaatgaggagtacggagaattctataagagcttgaccaatgactgggaagatcacttggcagtgaagcatttttcagttgaaggacagttggaattcagagcccttctatttgtcccacgacgtgctccttttgatctgtttgaaaacagaaagaaaaagaacaacatcaaattgtatgtacgcagagttttcatcatggataactgtgaggagctaatccctgaatatctgaacttcattagaggggtggtagactcggaggatctccctctaaacatatcccgtgagatgttgcaacaaagcaaaattttgaaagttatcaggaagaatttggtcaaaaaatgcttagaactctttactgaactggcggaagataaagagaactacaagaaattctatgagcagttctctaaaaacataaagcttggaatacacgaagactctcaaaatcggaagaagctttcagagctgttaaggtactacacatctgcctctggtgatgagatggtttctctcaaggactactgcaccagaatgaaggagaaccagaaacatatctattatatcacaggtgagaccaaggaccaggtagctaactcagcctttgtggaacgtcttcggaaacatggcttagaagtgatctatatgattgagcccattgatgagtactgtgtccaacagctgaaggaatttgaggggaagactttagtgtcagtcaccaaagaaggcctggaacttccagaggatgaagaagagaaaaagaagcaggaagagaaaaaaacaaagtttgagaacctctgcaaaatcatgaaagacatattggagaaaaaagttgaaaaggtggttgtgtcaaaccgattggtgacatctccatgctgtattgtcacaagcacatatggctggacagcaaacatggagagaatcatgaaagctcaagccctaagagacaactcaacaatgggttacatggcagcaaagaaacacctggagataaaccctgaccattccattattgagaccttaaggcaaaaggcagaggctgataagaacgacaagtctgtgaaggatctggtcatcttgctttatgaaactgcgctcctgtcttctggcttcagtctggaagatccccagacacatgctaacaggatctacaggatgatcaaacttggtctgggtattgatgaagatgaccctactgctgatgataccagtgctgctgtaactgaagaaatgccaccccttgaaggagatgacgacacatcacgcatggaagaagtagactaatctctggctgagggatgacttacctgttcagtactctacaattcctctgataatatattttcaaggatgtttttctttatttttgttaatattaaaaagtctgtatggcatgacaactactttaaggggaagataagatttctgtctactaagtgatgctgtgataccttaggcactaaagcagagctagtaatgctttttgagtttcatgttggtttattttcacagattggggtaacgtgcactgtaagacgtatgtaacatgatgttaactttgtggtctaaagtgtttagctgtcaagccggatgcctaagtagaccaaatcttgttattgaagtgttctgagctgtatcttgatgtttagaaaagtattcgttacatcttgtaggatctactttttgaacttttcattccctgtagttgacaattctgcatgtactagtcctctagaaataggttaaactgaagcaacttgatggaaggatctctccacagggcttgttttccaaagaaaagtattgtttggaggagcaaagttaaaagcctacctaagcatatcgtaaagctgttcaaaaataactcagacccagtcttgtggatggaaatgtagtgctcgagtcacattctgcttaaagttgtaacaaatacagatgagttaaaagatattgtgtgacagtgtcttatttagggggaaaggggagtatctggatgacagttagtgccaaaatgtaaaacatgaggcgctagcaggagatggttaaacactagctgctccaagggttgacatggtcttcccagcatgtactcagcaggtgtggggtggagcacacgtaggcacagaaaacaggaatgcagacaacatgcatcccctgcgtccatgagttacatgtgttctcttagtgtccacgttgttttgatgttattcatggaataccttctgtgttaaatacagtcacttaattccttggccttaaaa

SEQ ID NO: 48 - protein, 732 aa, Homo sapiens; identical to SEQ ID NO: 4

SEQ ID NO: 49 - DNA, 2792 bp, Cricetulus griseus
  CDS (28)..(2229): NM_001246821 WT, CDS 28-2229
cgtcccgtgcgcggtcactcagcgaagatgcctgaggaaacgcagacccaagaccagccaatggaggaggaagaggtggagacctttgcctttcaggcagagatcgcccagctgatgtccttgatcatcaacaccttctactccaacaaagagatctttctgagggagctcatttcaaactcgtcagatgctctggataagatcagatacgagagtctgacagaccctagtaaattagactctgggaaggagctgcacattaatattattcccaacaaacaagaccgaacccttactatcgtggatactggaattggaatgaccaaggccgacttgatcaataaccttggtaccattgccaagtctggcaccaaagccttcatggaggctttgcaggctggtgcagacatctctatgattggccagtttggtgttggtttttacactgcgtatttggttgccgagaaagtgactgtgatcaccaaacataacgatgatgagcagtacgcctgggagtcctcagccgggggctccttcacagtgaggactgacacaggtgaaccaatgggtcgtggaacaaaggttatcttacacctgaaagaagaccaaacggagtatatggaggaaaggagaataaaggagattgtgaagaaacattctcaatttatcggctatcccattactctcttcgtggagaaggaacgcgataaggaagtcagtgatgatgaagctgaagaaaaagaagataaagaggaagagaaggaaaaggaagaaaaaggaatcgacgacaaaccagaaatagaagatgtgggttctgatgaagaggaggaggaaaagaaggatggcgacaagaagaaaaagaagaagataaaggagaagtacattgaccaagaagagctcaacaagacaaagcctatttggaccaggaatcctgatgacattactaatgaagagtatggagagttctacaagagcttaaccaacgattgggaagaacatttggcagtgaagcatttttctgttgaaggacaattggaattccgggcccttctctttgtcccaagacgtgctccatttgatctgtttgaaaacagaaagaaaaagaacaacattaagttgtatgtgcgcagagttttcatcatggataactgtgaggagttattccctgagtatctaaatttcattagaggagtggtggattctgaggatcttcctctaaatatttcccgtgaaatattgcagcaaagcaaaattttgaaagttatcagaaagaatttggtcaggaaatgcttagaactatttcatgaactggcagaagacaaagagaactacaaaaagttttatgagcagttctctaaaaatataaagcttggaattcatgaagactctcaaaatcggaagaagctttcagagctgttgcgatactatacatctgcttctggagatgagatggtttctctcaaggactattgtaccagaatgaaggaaaaccagaagcatatctacttcatcacaggtgagaccaaggaccaagttgctaactcagccttcgtggaacgtcttcggaagcatggcttagaagtaatctatatgattgagcccattgatgagtactgtgtgcaacagctgaaggagtttgagggcaagaccttggtgtctgttaccaaagaaggactggaacttccagaagatgaagaggagaaaaagaaacaggaagagaaaaagaccaaatttgaaaacctctgcaagattatgaaagatattttggagaaaaaagttgagaaggtggtcgtatcaaaccgattggtgacgtccccttgctgtattgtcacaagcacatacgggtggacagcaaacatggagagaatcattaaagctcaagctctcagagacaactcaacgatgggttacatggcagcaaagaaacacctggagataaaccctgatcactccattattgaaaccctacgacaaaaggcagaggctgacaagaatgacaagtctgtgaaggatctggtgatcttgctgtatgaaactgcactcctgtcttctggtttcagtctggaagatccccagacacatgctaacaggatctacaggatgatcaagcttggtctaggtattgatgaagatgatcctactgtggatgataccagtgctgctgtaactgaagaaatgccacccctggaaggagatgacgacacatcacgcatggaagaagtcgactaagcttcaccagagctatatgtttgatgcttactttcattccttctgataatatattttcaaggatttttgtttgatttttgttaacatttaaaacatctgtgtggcatgttttctaaggggaagaaaaatttctttctacttgtgatactgtgatactctaggcttgactcaacgggttggtagtacatttgtgtaagacaaaatgtaacctattaacttgtggtctgcagtgtttagctgttgagctgattccttagtagaccaaatttgtcacttttggaagtgttctaagatagcttcatgtttaaaaggaaaaagattaagatgggcctaagtttcatcttaaaattttcatcccctgtagttaattctgcatgtactagtcttagaagtaaatacacaagctaaaacaactgaggaattccccatgtggcttgttttcaaagtactgagaacaaaactaagcttccctaggtcttgtaagtgagcaaggcttgtgaatggaaatatagtgcccaatcacattctgctttaaaaggtaaatacagatgagataaaaacct

SEQ ID NO: 50 - protein, 733 aa, Cricetulus griseus; identical to SEQ ID NO: 6

SEQ ID NO: 51 - DNA, 2525 bp, Homo sapiens
  CDS (96)..(2240): NM_001271972 WT, CDS 96-2240
ctctcgagtcactccggcgcagtgttgggactgtctgggtatcggaaagcaagcctacgttgctcactattacgtataatccttttcttttcaagatgcctgaggaagtgcaccatggagaggaggaggtggagacttttgcctttcaggcagaaattgcccaactcatgtccctcatcatcaataccttctattccaacaaggagattttccttcgggagttgatctctaatgcttctgatgccttggacaagattcgctatgagagcctgacagacccttcgaagttggacagtggtaaagagctgaaaattgacatcatccccaaccctcaggaacgtaccctgactttggtagacacaggcattggcatgaccaaagctgatctcataaataatttgggaaccattgccaagtctggtactaaagcattcatggaggctcttcagtttggtgttggcttttattctgcctacttggtggcagagaaagtggttgtgatcacaaagcacaacgatgatgaacagtatgcttgggagtcttctgctggaggttccttcactgtgcgtgctgaccatggtgagcccattggcaggggtaccaaagtgatcctccatcttaaagaagatcagacagagtacctagaagagaggcgggtcaaagaagtagtgaagaagcattctcagttcataggctatcccatcaccctttatttggagaaggaacgagagaaggaaattagtgatgatgaggcagaggaagagaaaggtgagaaagaagaggaagataaagatgatgaagaaaaacccaagatcgaagatgtgggttcagatgaggaggatgacagcggtaaggataagaagaagaaaactaagaagatcaaagagaaatacattgatcaggaagaactaaacaagaccaagcctatttggaccagaaaccctgatgacatcacccaagaggagtatggagaattctacaagagcctcactaatgactgggaagaccacttggcagtcaagcacttttctgtagaaggtcagttggaattcagggcattgctatttattcctcgtcgggctccctttgacctttttgagaacaagaagaaaaagaacaacatcaaactctatgtccgccgtgtgttcatcatggacagctgtgatgagttgataccagagtatctcaattttatccgtggtgtggttgactctgaggatctgcccctgaacatctcccgagaaatgctccagcagagcaaaatcttgaaagtcattcgcaaaaacattgttaagaagtgccttgagctcttctctgagctggcagaagacaaggagaattacaagaaattctatgaggcattctctaaaaatctcaagcttggaatccacgaagactccactaaccgccgccgcctgtctgagctgctgcgctatcatacctcccagtctggagatgagatgacatctctgtcagagtatgtttctcgcatgaaggagacacagaagtccatctattacatcactggtgagagcaaagagcaggtggccaactcagcttttgtggagcgagtgcggaaacggggcttcgaggtggtatatatgaccgagcccattgacgagtactgtgtgcagcagctcaaggaatttgatgggaagagcctggtctcagttaccaaggagggtctggagctgcctgaggatgaggaggagaagaagaagatggaagagagcaaggcaaagtttgagaacctctgcaagctcatgaaagaaatcttagataagaaggttgagaaggtgacaatctccaatagacttgtgtcttcaccttgctgcattgtgaccagcacctacggctggacagccaatatggagcggatcatgaaagcccaggcacttcgggacaactccaccatgggctatatgatggccaaaaagcacctggagatcaaccctgaccaccccattgtggagacgctgcggcagaaggctgaggccgacaagaatgataaggcagttaaggacctggtggtgctgctgtttgaaaccgccctgctatcttctggcttttcccttgaggatccccagacccactccaaccgcatctatcgcatgatcaagctaggtctaggtattgatgaagatgaagtggcagcagaggaacccaatgctgcagttcctgatgagatcccccctctcgagggcgatgaggatgcgtctcgcatggaagaagtcgattaggttaggagttcatagttggaaaacttgtgcccttgtatagtgtccccatgggctcccactgcagcctcgagtgcccctgtcccacctggctccccctgctggtgtctagtgtttttttccctctcctgtccttgtgttgaaggcagtaaactaagggtgtcaagccccattccctctctactcttgacagcaggattggatgttgtgtattgtggtttattttattttcttcattttgttctgaaattaaagtatgcaaaataaagaatatgccgtttttataca

SEQ ID NO: 52 - protein, 714 aa, Homo sapiens; identical to SEQ ID NO: 12

SEQ ID NO: 53 - DNA, 2611 bp, Homo sapiens
  CDS (152)..(2326): NM_001271970 WT, CDS 152-2326
actctcagtctgcaaaagtgtacgcccgcagagccgccccaggtgcctgggtgttgtgtgattgacgcggggaaggaggggtcagccgatccctccccaaccctccatcccatccctgaggattgggctggtacccgcgtctctcggacagatgcctgaggaagtgcaccatggagaggaggaggtggagacttttgcctttcaggcagaaattgcccaactcatgtccctcatcatcaataccttctattccaacaaggagattttccttcgggagttgatctctaatgcttctgatgccttggacaagattcgctatgagagcctgacagacccttcgaagttggacagtggtaaagagctgaaaattgacatcatccccaaccctcaggaacgtaccctgactttggtagacacaggcattggcatgaccaaagctgatctcataaataatttgggaaccattgccaagtctggtactaaagcattcatggaggctcttcaggctggtgcagacatctccatgattgggcagtttggtgttggcttttattctgcctacttggtggcagagaaagtggttgtgatcacaaagcacaacgatgatgaacagtatgcttgggagtcttctgctggaggttccttcactgtgcgtgctgaccatggtgagcccattggcaggggtaccaaagtgatcctccatcttaaagaagatcagacagagtacctagaagagaggcgggtcaaagaagtagtgaagaagcattctcagttcataggctatcccatcaccctttatttggagaaggaacgagagaaggaaattagtgatgatgaggcagaggaagagaaaggtgagaaagaagaggaagataaagatgatgaagaaaaacccaagatcgaagatgtgggttcagatgaggaggatgacagcggtaaggataagaagaagaaaactaagaagatcaaagagaaatacattgatcaggaagaactaaacaagaccaagcctatttggaccagaaaccctgatgacatcacccaagaggagtatggagaattctacaagagcctcactaatgactgggaagaccacttggcagtcaagcacttttctgtagaaggtcagttggaattcagggcattgctatttattcctcgtcgggctccctttgacctttttgagaacaagaagaaaaagaacaacatcaaactctatgtccgccgtgtgttcatcatggacagctgtgatgagttgataccagagtatctcaattttatccgtggtgtggttgactctgaggatctgcccctgaacatctcccgagaaatgctccagcagagcaaaatcttgaaagtcattcgcaaaaacattgttaagaagtgccttgagctcttctctgagctggcagaagacaaggagaattacaagaaattctatgaggcattctctaaaaatctcaagcttggaatccacgaagactccactaaccgccgccgcctgtctgagctgctgcgctatcatacctcccagtctggagatgagatgacatctctgtcagagtatgtttctcgcatgaaggagacacagaagtccatctattacatcactggtgagagcaaagagcaggtggccaactcagcttttgtggagcgagtgcggaaacggggcttcgaggtggtatatatgaccgagcccattgacgagtactgtgtgcagcagctcaaggaatttgatgggaagagcctggtctcagttaccaaggagggtctggagctgcctgaggatgaggaggagaagaagaagatggaagagagcaaggcaaagtttgagaacctctgcaagctcatgaaagaaatcttagataagaaggttgagaaggtgacaatctccaatagacttgtgtcttcaccttgctgcattgtgaccagcacctacggctggacagccaatatggagcggatcatgaaagcccaggcacttcgggacaactccaccatgggctatatgatggccaaaaagcacctggagatcaaccctgaccaccccattgtggagacgctgcggcagaaggctgaggccgacaagaatgataaggcagttaaggacctggtggtgctgctgtttgaaaccgccctgctatcttctggcttttcccttgaggatccccagacccactccaaccgcatctatcgcatgatcaagctaggtctaggtattgatgaagatgaagtggcagcagaggaacccaatgctgcagttcctgatgagatcccccctctcgagggcgatgaggatgcgtctcgcatggaagaagtcgattaggttaggagttcatagttggaaaacttgtgcccttgtatagtgtccccatgggctcccactgcagcctcgagtgcccctgtcccacctggctccccctgctggtgtctagtgtttttttccctctcctgtccttgtgttgaaggcagtaaactaagggtgtcaagccccattccctctctactcttgacagcaggattggatgttgtgtattgtggtttattttattttcttcattttgttctgaaattaaagtatgcaaaataaagaatatgccgtttttataca

SEQ ID NO: 54 - protein, 724 aa, Homo sapiens; identical to SEQ ID NO: 8

SEQ ID NO: 55 - DNA, 2411 bp, Homo sapiens
  CDS (96)..(2126): NM_001271971 WT, CDS 96-2126
ctctcgagtcactccggcgcagtgttgggactgtctgggtatcggaaagcaagcctacgttgctcactattacgtataatccttttcttttcaagatgcctgaggaagtgcaccatggagaggaggaggtggagacttttgcctttcaggcagaaattgcccaactcatgtccctcatcatcaataccttctattccaacaaggagattttccttcgggagttgatctctaatgcttctgatgccttggacaagattcgctatgagagcctgacagacccttcgaagttggacagtggtaaagagctgaaaattgacatctccatgattgggcagtttggtgttggcttttattctgcctacttggtggcagagaaagtggttgtgatcacaaagcacaacgatgatgaacagtatgcttgggagtcttctgctggaggttccttcactgtgcgtgctgaccatggtgagcccattggcaggggtaccaaagtgatcctccatcttaaagaagatcagacagagtacctagaagagaggcgggtcaaagaagtagtgaagaagcattctcagttcataggctatcccatcaccctttatttggagaaggaacgagagaaggaaattagtgatgatgaggcagaggaagagaaaggtgagaaagaagaggaagataaagatgatgaagaaaaacccaagatcgaagatgtgggttcagatgaggaggatgacagcggtaaggataagaagaagaaaactaagaagatcaaagagaaatacattgatcaggaagaactaaacaagaccaagcctatttggaccagaaaccctgatgacatcacccaagaggagtatggagaattctacaagagcctcactaatgactgggaagaccacttggcagtcaagcacttttctgtagaaggtcagttggaattcagggcattgctatttattcctcgtcgggctccctttgacctttttgagaacaagaagaaaaagaacaacatcaaactctatgtccgccgtgtgttcatcatggacagctgtgatgagttgataccagagtatctcaattttatccgtggtgtggttgactctgaggatctgcccctgaacatctcccgagaaatgctccagcagagcaaaatcttgaaagtcattcgcaaaaacattgttaagaagtgccttgagctcttctctgagctggcagaagacaaggagaattacaagaaattctatgaggcattctctaaaaatctcaagcttggaatccacgaagactccactaaccgccgccgcctgtctgagctgctgcgctatcatacctcccagtctggagatgagatgacatctctgtcagagtatgtttctcgcatgaaggagacacagaagtccatctattacatcactggtgagagcaaagagcaggtggccaactcagcttttgtggagcgagtgcggaaacggggcttcgaggtggtatatatgaccgagcccattgacgagtactgtgtgcagcagctcaaggaatttgatgggaagagcctggtctcagttaccaaggagggtctggagctgcctgaggatgaggaggagaagaagaagatggaagagagcaaggcaaagtttgagaacctctgcaagctcatgaaagaaatcttagataagaaggttgagaaggtgacaatctccaatagacttgtgtcttcaccttgctgcattgtgaccagcacctacggctggacagccaatatggagcggatcatgaaagcccaggcacttcgggacaactccaccatgggctatatgatggccaaaaagcacctggagatcaaccctgaccaccccattgtggagacgctgcggcagaaggctgaggccgacaagaatgataaggcagttaaggacctggtggtgctgctgtttgaaaccgccctgctatcttctggcttttcccttgaggatccccagacccactccaaccgcatctatcgcatgatcaagctaggtctaggtattgatgaagatgaagtggcagcagaggaacccaatgctgcagttcctgatgagatcccccctctcgagggcgatgaggatgcgtctcgcatggaagaagtcgattaggttaggagttcatagttggaaaacttgtgcccttgtatagtgtccccatgggctcccactgcagcctcgagtgcccctgtcccacctggctccccctgctggtgtctagtgtttttttccctctcctgtccttgtgttgaaggcagtaaactaagggtgtcaagccccattccctctctactcttgacagcaggattggatgttgtgtattgtggtttattttattttcttcattttgttctgaaattaaagtatgcaaaataaagaatatgccgtttttataca

SEQ ID NO: 56 - protein, 676 aa, Homo sapiens; identical to SEQ ID NO: 10

SEQ ID NO: 57 - DNA, 2414 bp, Cricetulus griseus
  CDS (30)..(2204): XM_003501668.2 WT, CDS 30-2204
ttagtaattaatgtgtaatgctttcttagatgcctgaggaggtgcaccatggcgaggaagaggtggagacctttgccttccaggcagaaatcgcccagctcatgtccttgatcataaatactttctactcaaacaaggagattttccttcgagaattgatctccaatgcttctgatgccttggacaagattcgctatgagagcctgacagacccttccaaattggacagcggcaaagagctgaagattgacatcatccccaaccctcaggagcgcacgctgactttggtggacacgggcattggcatgaccaaagctgatctcataaataacttgggaaccattgctaagtctggcacaaaggctttcatggaggctctccaggctggtgcagacatatccatgattgggcagtttggtgttggattctactctgcctatctcgtggcagagaaagtggttgtgatcacaaagcacaatgatgacgagcagtatgcttgggagtcctctgctggtggatccttcacagtgcgtgcagaccatggtgagcccattggccggggtaccaaagtcatccttcatctcaaagaagaccagacagagtacttagaggagaggagggtcaaggaagtggtgaagaaacactcgcagttcataggctatcccatcaccctctatttggagaaggaacgagagaaggagatcagtgatgatgaggcagaagaagagaagggtgagaaagaggaagaagataaggatgatgaggaaaagccaaagattgaagatgtaggatcagatgaggaggatgacagtggcaaagataagaaaaagaaaacgaagaagattaaggagaaatacattgatcaggaagagttgaataagactaagcccatttggaccagaaaccctgatgacatcactcaagaagaatatggtgatttctataagagcctcaccaatgattgggaggaccacttggcagtcaagcacttctctgtagagggtcagttggaattcagagcattgctcttcattcctcggcgagctccctttgacctttttgagaacaagaagaagaagaacaatatcaaactgtacgtccgtcgtgtgttcatcatggacagctgtgatgagctgatccccgagtacctcaactttatccgtggcgtggtggactctgaggacctgcccctgaacatctctcgagaaatgctccaacagagcaagatcctgaaagttatccgtaaaaacattgtgaagaagtgccttgagctcttctctgagctggctgaagacaaagagaactataagaaattctacgaggccttctccaagaacttaaagcttggaatccatgaagattccactaaccgtcggcgtctctctgagctgctgcgctatcatacctctcagtctggagatgagatgacttccctgtcagaatatgtgtcccgcatgaaggagacacagaagtccatctattacatcactggtgagagcaaagagcaggtagccaactctgcctttgtggagcgtgtgccgaagcgaggctttgaagcggtgtacatgactgagcctattgatgagtactgcgtgcaacagctcaaggagtttgatggaaagagtctggtctcagtgactaaggaaggcctggagctgccagaagatgaggaagagaagaagaaaatggaggagagcaaggcaaaatttgagaatctctgcaagctcatgaaggagatcttggataagaaggttgagaaggtgaccatctccaataggctagtgtcttcaccctgctgcattgtgacaagcacctatggctggacagccaacatggagcggatcatgaaggcccaggcactcagagacaactctaccatgggctacatgatggccaaaaagcacctagagatcaaccctgaccaccccatcgtggagactctgaggcagaaggctgaggcagataaaaacgacaaagctgtcaaggatctggtggtgctgctgtttgagactgctctgctctcctctggtttctcacttgaggatccccaaacccactccaaccgcatctaccgcatgatcaaactaggcctgggtatcgatgaagatgaggtgacagcagaggagcccagtgctgctgttcctgatgagatccccccactcgagggtgatgaggatgcctcccgcatggaagaagtagattagagatcccctgggaagaccatgtcctctgtatagtttccctgagccccaccccccatgccttggctggccccaacctacctggctctctgttcaagctagaggatctctttatcctgtccttgtgccttaaggcaggaaagtcccctcccacagaatagcagggttgggtattgtgtattgtggttttgtttgttttattttgttctgaaa

SEQ ID NO: 58 - protein, 724 aa, Cricetulus griseus; identical to SEQ ID NO: 14

SEQ ID NO: 59 - DNA, 1618 bp, Homo sapiens
  CDS (64)..(1200): NM_007065 WT, CDS 64-1200
agtctccactgctgccgccgtcgccgccacccgagccggagcgggctgggccgccaaggcaagatggtggactacagcgtgtgggaccacattgaggtgtctgatgatgaagacgagacgcaccccaacatcgacacggccagtctcttccgctggcggcatcaggcccgggtggaacgcatggagcagttccagaaggagaaggaggaactggacaggggctgccgcgagtgcaagcgcaaggtggccgagtgccagaggaaactgaaggagctggaggtggccgagggcggcaaggcagagctggagcgcctgcaggccgaggcacagcagctgcgcaaggaggagcggagctgggagcagaagctggaggagatgcgcaagaaggagaagagcatgccctggaacgtggacacgctcagcaaagacggcttcagcaagagcatggtaaataccaagcccgagaagacggaggaggactcagaggaggtgagggagcagaaacacaagaccttcgtggaaaaatacgagaaacagatcaagcactttggcatgcttcgccgctgggatgacagccaaaagtacctgtcagacaacgtccacctggtgtgcgaggagacagccaattacctggtcatttggtgcattgacctagaggtggaggagaaatgtgcactcatggagcaggtggcccaccagacaatcgtcatgcaatttatcctggagctggccaagagcctaaaggtggacccccgggcctgcttccggcagttcttcactaagattaagacagccgatcgccagtacatggagggcttcaacgacgagctggaagccttcaaggagcgtgtgcggggccgtgccaagctgcgcatcgagaaggccatgaaggagtacgaggaggaggagcgcaagaagcggctcggccccggcggcctggaccccgtcgaggtctacgagtccctccctgaggaactccagaagtgcttcgatgtgaaggacgtgcagatgctgcaggacgccatcagcaagatggaccccaccgacgcaaagtaccacatgcagcgctgcattgactctggcctctgggtccccaactctaaggccagcgaggccaaggagggagaggaggcaggtcctggggacccattactggaagctgttcccaagacgggcgatgagaaggatgtcagtgtgtgacctgccccagctaccaccgccacctgcttccaggcccctatgtgccccttttcagaaaacagatagatgccatctcgcccgctcctgacttcctctacttgcgctgctcggcccagcctggggggcccgcccagccctccctggcctctccactgtctccactctccagcgcccattcaagtctctgctttgagtcaaggggcttcactgcctgcagccccccatcagcattatgccaaaggcccgggggtccggggaagggcagaggtcaccaggctggtctaccaggtagttggggagggtccccagccaaggggccggctctcgtcactgggctctgttttcactgttcgtctgctgtctgtgtcttctatttggcaaacagcaatgatcttccaataaaagatttcagatgctc

SEQ ID NO: 60 - protein, 378 aa, Homo sapiens; identical to SEQ ID NO: 16

SEQ ID NO: 61 - DNA, 1250 bp, Cricetulus griseus
  CDS (111)..(1250): XM_003499737 WT, CDS 111-1250
gaagcggaaggggcggggctccgcgagcggactctctatcccttgtgggtccctgttcagcgcccggccgtgtggagcgccgggagccaatccgggcctcctaggcgaagatggtggactacagcgtttgggatcacatcgaggtgtcggacgatgaggacgagacgcaccccaacatcgacacggccagcctcttccgctggcggcaccaggcccgagtggagcgcatggagcagtttcagaaggagaaggacgaactggaccggggctgccgtgaatgcaagcgaaaggtagccgaatgccaacgcaagctgagggagctggaagtggctgaaggcggtggccaggtggagcttgagcggctgcgagctgaggcacagcagctgcgcaaggaggagcgaagctgggagcagaagctggaggacatgcgcaagaaggagaagagcatgccctggaacgtggacaccctcagcaaggacggcttcagcaagagcatagtcaatatcaagcctgagaaggcagaggaagactcagaggaggcaagagagcagaaacacaagaccttcgttgaaaagtacgagaaacagatcaagcatttcggcatgctccaccgctgggatgacagccagaagtacctgtcagacaatgtccacctggtgtgcgaggagaccgccaactacctggttatctggtgcattgacctggaagtggaggagaaatgtgcactgatggagcaggtagctcaccagaccatggtgatgcagttcattctggagctggccaagagtctgaaggtcgatccccgtgcctgcttcaggcagttcttcaccaagatcaagaccgccgaccaacagtacatggagggcttcaaatacgaactggaggcctttaaggagcgtgtgcggggccgtgccaagctgcgcatagagaaggccatgaaggagtacgaggaggaggagcgcaagaagaggctgggccctggtggcctggaccccgtggaggtctatgagtccctgcctgaggaactgcagaagtgctttgatgtgaaggacgtgcaaatgctgcaagatgccatcagcaagatggatcccactgatgccaagtttcacatgcagcgttgcatcgattctgggctctgggtccccaactccaagtctggtgaggccaaggagggggaggaggcgggccccggggacccattgctggaagccgtccccaaagcgggcaacgagaaagacgtcagtgcgtga

SEQ ID NO: 62 - protein, 379 aa, Cricetulus griseus; identical to SEQ ID NO: 18

SEQ ID NO: 63 - DNA, 2301 bp, Homo sapiens
  CDS (118)..(1839): NM_199440 WT, CDS 118-1839
gccccgacgcgcaccgcgattcgccccaagggccctgcgcaggacgctgacgcgaagactcggaggcggaagaaaaaaggagctgtttctaggcttttctaggcgcccagccgagaaatgcttcggttacccacagtctttcgccagatgagaccggtgtccagggtactggctcctcatctcactcgggcttatgccaaagatgtaaaatttggtgcagatgcccgagccttaatgcttcaaggtgtagaccttttagccgatgctgtggccgttacaatggggccaaagggaagaacagtgattattgagcagagttggggaagtcccaaagtaacaaaagatggtgtgactgttgcaaagtcaattgacttaaaagataaatacaaaaacattggagctaaacttgttcaagatgttgccaataacacaaatgaagaagctggggatggcactaccactgctactgtactggcacgctctatagccaaggaaggcttcgagaagattagcaaaggtgctaatccagtggaaatcaggagaggtgtgatgttagctgttgatgctgtaattgctgaacttaaaaagcagtctaaacctgtgaccacccctgaagaaattgcacaggttgctacgatttctgcaaacggagacaaagaaattggcaatatcatctctgatgcaatgaaaaaagttggaagaaagggtgtcatcacagtaaaggatggaaaaacactgaatgatgaattagaaattattgaaggcatgaagtttgatcgaggctatatttctccatactttattaatacatcaaaaggtcagaaatgtgaattccaggatgcctatgttctgttgagtgaaaagaaaatttctagtatccagtccattgtacctgctcttgaaattgccaatgctcaccgtaagcctttggtcataatcgctgaagatgttgatggagaagctctaagtacactcgtcttgaataggctaaaggttggtcttcaggttgtggcagtcaaggctccagggtttggtgacaatagaaagaaccagcttaaagatatggctattgctactggtggtgcagtgtttggagaagagggattgaccctgaatcttgaagacgttcagcctcatgacttaggaaaagttggagaggtcattgtgaccaaagacgatgccatgctcttaaaaggaaaaggtgacaaggctcaaattgaaaaacgtattcaagaaatcattgagcagttagatgtcacaactagtgaatatgaaaaggaaaaactgaatgaacggcttgcaaaactttcagatggagtggctgtgctgaaggttggtgggacaagtgatgttgaagtgaatgaaaagaaagacagagttacagatgcccttaatgctacaagagctgctgttgaagaaggcattgttttgggagggggttgtgccctccttcgatgcattccagccttggactcattgactccagctaatgaagatcaaaaaattggtatagaaattattaaaagaacactcaaaattccagcaatgaccattgctaagaatgcaggtgttgaaggatctttgatagttgagaaaattatgcaaagttcctcagaagttggttatgatgctatggctggagattttgtgaatatggtggaaaaaggaatcattgacccaacaaaggttgtgagaactgctttattggatgctgctggtgtggcctctctgttaactacagcagaagttgtagtcacagaaattcctaaagaagagaaggaccctggaatgggtgcaatgggtggaatgggaggtggtatgggaggtggcatgttctaactcctagactagtgctttacctttattaatgaactgtgacaggaagcccaaggcagtgttcctcaccaataacttcagagaagtcagttggagaaaatgaagaaaaaggctggctgaaaatcactataaccatcagttactggtttcagttgacaaaatatataatggtttactgctgtcattgtccatgcctacagataatttattttgtatttttgaataaaaaacatttgtacattcctgatactgggtacaagagccatgtaccagtgtactgctttcaacttaaatcactgaggcatttttactactattctgttaaaatcaggattttagtgcttgccaccaccagatgagaagttaagcagcctttctgtggagagtgagaataattgtgtacaaagtagagaagtatccaattatgtgacaacctttgtgtaataaaaatttgtttaaagttaa

SEQ ID NO: 64 - protein, 573 aa, Homo sapiens; identical to SEQ ID NO: 20

SEQ ID NO: 65 - DNA, 2319 bp, Homo sapiens
  CDS (122)..(1315): NM_001539 WT, CDS 122-1315
gcggaactttccagaacgctcggtgagaggcggaggagcggtaactaccccggctgcgcacagctcggcgctccttcccgctccctcacacaccggcctcagcccgcaccggcagtagaagatggtgaaagaaacaacttactacgatgttttgggggtcaaacccaatgctactcaggaagaattgaaaaaggcttataggaaactggctttgaagtaccatcctgataagaacccaaatgaaggagagaagtttaaacagatttctcaagcttacgaagttctctctgatgcaaagaaaagggaattatatgacaaaggaggagaacaggcaattaaagagggtggagcaggtggcggttttggctcccccatggacatctttgatatgttttttggaggaggaggaaggatgcagagagaaaggagaggtaaaaatgttgtacatcagctctcagtaaccctagaagacttatataatggtgcaacaagaaaactggctctgcaaaagaatgtgatttgtgacaaatgtgaaggtagaggaggtaagaaaggagcagtagagtgctgtcccaattgccgaggtactggaatgcaaataagaattcatcagataggacctggaatggttcagcaaattcagtctgtgtgcatggagtgccagggccatggggagcggatcagtcctaaagatagatgtaaaagctgcaacggaaggaagatagttcgagagaagaaaattttagaagttcatattgacaaaggcatgaaagatggccagaagataacattccatggtgaaggagaccaagaaccaggactggagccaggcgatattatcattgtgttagatcagaaggaccatgctgtttttactcgacgaggagaagaccttttcatgtgtatggacatacagctcgttgaagcactgtgtggcttccagaagccaatatctactcttgacaaccgaaccatcgtcatcacctctcatccaggtcagattgtcaagcatggagatatcaagtgtgtactaaatgaaggcatgccaatttatcgtagaccatatgaaaagggtcgcctaatcatcgaatttaaggtaaactttcctgagaatggctttctctctcctgataaactgtctttgctggaaaaactcctacccgagaggaaggaagtggaagagactgatgagatggaccaagtagaactggtggactttgatccaaatcaggaaagacggcgccactacaatggagaagcatatgaggatgatgaacatcatcccagaggtggtgttcagtgtcagacctcttaatgggccagtgaataacactcactgctggcatttaatgtgcagtagtgaatgagtgaaggactgtaatcataatatgctcactacttgctcttgtttttgttttaataaactatagtagtgttttaaaaagttaaatgaagaataaacgcaaatataaaagctctgattttgccctgtatgtatgatgacttcagtgtgcaagatgaagtttaatacctgtaaaaactacaaagaagttcccctagcatttctaggccaaaccttgtaattgacttcagctatgtacgtggacaagcttagactgaaatgctaggtatatgtattggcttcagtgtatgacccttcattgttaagctatgaaagtaaaactctgtatttaactggcaatgaggaaaaaaaaattttgtagagaagtgttggtctgtatagttctttatattaagtgggattcattgtaatgcctctgcatttattctgttgcctcagctgttacttgaagatggcgtaatatataatttatcctgtggtatcagtgataaaaatgatacctttctgtaggaggggtttatcataatatgctgcttcttgaaggcttgcacttccagaattgtgtttccttctgctgtgccattcatatatatatacatatatatatataatcttgaccagtcctggtcatttgctcccctccttgtctgtggaccatgataagcccaagtagtgacttcagagctgggtaacagaaattaaagtgaaaagacctttacgtggagaatttgcatgcgtaatataggaaggtgttctttaggtatgttacaggattactttaaaccatttgactttcgctccaaagttatgttggtagtatagcaaattatgatgaatagctttaattgtatgtttaaaagtctcatatgttcacatgcttaaatctgggtatcagaatttaagcaattcttgaaatgtattgtctccttaatatactaattacaaagcatctccaa

SEQ ID NO: 66 - protein, 397 aa, Homo sapiens; identical to SEQ ID NO: 22

SEQ ID NO: 67 - DNA, 557 bp, Homo sapiens
  CDS (59)..(367): NM_002157 WT, CDS 59-367
gcgagtctctttgcggcgctacactagagcagagtacgagtctgaggcggagggagtaatggcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactgaaataagtcactattgaaatggcatcaacatgatgctgcccattccactgaagttctgaaatctttcgtcatgtaaataatttccatatttctcttttataataaactaatgataactaatgacatccagtgtctccaaaattgtttccttgtactgatataaacacttccaaataaaaatatgtaaatga

SEQ ID NO: 68 - protein, 102 aa, Homo sapiens; identical to SEQ ID NO: 24

SEQ ID NO: 69 - DNA, 5244 bp, Homo sapiens
  CDS (323)..(2899): NM_006644 WT, CDS 323-2899
gggtcggtagttcagcgccggcgccggtgtgcgagccgcggcagagtgaggcaggcaacccgaggtgcggagcgacctgcggaggctgagccccgctttctcccagggtttcttatcagccagccgccgctgtccccgggggagtaggaggctcctgacaggccgcggctgtctgtgtgtccttctgagtgtcagaggaacggccagaccccgcgggccggagcagaacgcggccagggcagaaagcggcggcaggagaagcaggcagggggccggaggacgcagaccgagacccgaggcggaggcggaccgcgagccggccatgtcggtggtggggttggacgtgggctcgcagagctgctacatcgcggtagcccgggccgggggcatcgagaccatcgccaatgagttcagcgaccggtgcaccccgtcagtcatatcatttggatcaaaaaatagaacaatcggagttgcagccaaaaatcagcaaatcactcatgcaaacaatacggtgtctaacttcaaaagatttcatggccgagcattcaatgaccccttcattcaaaaggagaaggaaaacttgagttacgatttggttccattgaaaaatggtggagttggaataaaggtaatgtacatgggtgaagaacatctatttagtgtggagcagataacagccatgttgttgactaagctgaaggaaactgctgaaaacagcctcaagaaaccagtaacagattgtgttatttcagtcccctccttctttacagatgctgagaggcgatctgtgttagatgctgcacagattgttggcctaaactgtttaagacttatgaatgacatgacagctgttgctttgaattacggaatttataagcaggatctcccaagcctggatgagaaacctcggatagtggtttttgttgatatgggacattcagcttttcaagtgtctgcttgtgcttttaacaagggaaaattgaaggtactgggaacagcttttgatcctttcttaggaggaaaaaacttcgatgaaaagttagtggaacatttttgtgcagaatttaaaactaagtacaagttggatgcaaaatccaaaatacgagcactcctacgtctgtatcaggaatgtgaaaaactgaaaaagctaatgagctctaacagcacagaccttccactgaatatcgaatgctttatgaatgataaagatgtttccggaaagatgaacaggtcacaatttgaagaactctgtgctgaacttctgcaaaagatagaagtacccctttattcactgttggaacaaactcatctcaaagtagaagatgtgagtgcagttgagattgttggaggcgctacacgaattccagctgtgaaggaaagaattgccaaattctttggaaaagatattagcacaacactcaatgcagatgaagcagtagccagaggatgtgcattacagtgtgcaatactttccccggcatttaaagttagagaattttccgtcacagatgcagttccttttccaatatctctgatctggaaccatgattcagaagatactgaaggtgttcatgaagtctttagtcgaaaccatgctgctcctttctccaaagttctcacctttctgagaagggggccttttgagctagaagctttctattctgatccccaaggagttccatatccagaagcaaaaataggccgctttgtagttcagaatgtttctgcacagaaagatggagaaaaatctagagtaaaagtcaaagtgcgagtcaacacccatggcattttcaccatctctacggcatctatggtggagaaagtcccaactgaggagaatgaaatgtcttctgaagctgacatggagtgtctgaatcagagaccaccagaaaacccagacactgataaaaatgtccagcaagacaacagtgaagctggaacacagccccaggtacaaactgatgctcaacaaacctcacagtctcccccttcacctgaacttacctcagaagaaaacaaaatcccagatgctgacaaagcaaatgaaaaaaaagttgaccagcctccagaagctaaaaagcccaaaataaaggtggtgaatgttgagctgcctattgaagccaacttggtctggcagttagggaaagaccttcttaacatgtatattgagacagagggtaagatgataatgcaagataaattggaaaaagaaaggaatgatgctaaaaatgcagttgaggaatatgtgtatgagttcagagacaagctgtgtggaccatatgaaaaatttatatgtgagcaggatcatcaaaattttttgagactcctcacagaaactgaagactggctgtatgaagaaggagaggaccaagctaaacaagcatatgttgacaagttggaagaattaatgaaaattggcactccagttaaagttcggtttcaggaagctgaagaacggccaaaaatgtttgaagaactaggacagaggctgcagcattatgccaagatagcagctgacttcagaaataaggatgagaaatacaaccatattgatgagtctgaaatgaaaaaagtggagaagtctgttaatgaagtgatggaatggatgaataatgtcatgaatgctcaggctaaaaagagtcttgatcaggatccagttgtacgtgctcaggaaattaaaacaaaaatcaaggaattgaacaacacatgtgaacccgttgtaacacaaccgaaaccaaaaattgaatcacccaaactggaaagaactccaaatggcccaaatattgataaaaaggaagaagatttagaagacaaaaacaattttggtgctgaacctccacatcagaatggtgaatgttaccctaatgagaaaaattctgttaatatggacttggactagataaccttaaattggcctattccttcaattaataaaatatttttgccatagtatgtgactctacataacatactgaaactatttatattttcttttttaaggatatttagaaattttgtgtattatatggaaaaagaaaaaaagcttaagtctgtagtctttatgatcctaaaagggaaaattgccttggtaactttcagattcctgtggaattgtgaattcatactaagctttctgtgcagtctcaccatttgcatcactgaggatgaaactgacttttgtcttttggagaaaaaaaactgtactgcttgttcaagagggctgtgattaaaatctttaagcatttgttcctgccaaggtagttttcttgcattttgctctccattcagcatgtgtgtgggtgtggatgtttataaacaagactaagtctgacttcataagggctttctaaaaccatttctgtccaagagaaaatgactttttgctttgatattaaaaattcaatgagtaaaacaaaagctagtcaaatgtgttagcagcatgcagaacaaaaactttaaactttctctctcactatacagtatattgtcatgtgaaagtgtggaatggaagaaatgtcgatcctgttgtaactgattgtgaacacttttatgagctttaaaataaagttcatcttatggtgtcatttctaaactgttgattttgtcactaatttaaaaaatgagatgagggagaatatgaattattctagcagaaatgaagttagtgtccagtttttcttttttactgcttatgttctctcttttctaagtgaaaatgtttttctcctgacagaaaaatagcatatgtttattacattaaagcattttaaaaatactataaagtgaataaaacttaaaatcttgccacccagaagaaaacattgttaacacttcgttatacatccttggagccttttacccctaatgatgggggtatatttgtgtgagtgtctatgttatagtgctgtaggctgtttttaaaaactctgtgccagggaaattagtaaatacagtcaagtgttgtaactgcataaatgtagtgagttcattataaagacgtacttaaaaataccaatcttgctattggtagatttaggtgtttctcatttttgctgttctaaataatgctgcaatgaatctatctggttgttttgtaagcacaaaattgaagtagtgttggaggtaggggagaagacacatgcacttaggataggtgacaaccagctcctcactagggtgcaacagtttattctttttttttttttggtagagctggggtttcgctatgttgcccaagttcgtcttgaactcctgggctcaagcaatctgtctgcctcagcctcccaaagtgctgggattacatggtgtgagccaccatgcccagccatttatgatcttgatgagggccaactttccctcttccacaagggattaatcttagaactatagccaatttattataagtactatttagctactctgtgctccacacatcacggagtaatatcttcaaactatgaggtttagtaacacttgactgctgaggtttatttcctcagctctgatgcttcctgggtgaagatgtaagttattaaattaactcaaattttaaagctaacacttcattccatctagaccaggtagctcgtcatttgtacgtctgataggtgcttcaacatctcagaattggcttatttttcatgcattgcatattgtactcatttgtaatattggaatgtaaaatgttcttactaagaatctttatgtcagctattcaacatttccctactttataatttctagctgctttaccatttagcaactttaaatttttatgcagttaatatgtttctttggtttttatgttttctgaatgtctttaatgcacattaatgtatacttagtagatacacatttacacattgagaagtgaagataggtttgtgttctgtgtacaattaggacatccattaaaaaagatttttaaaaactgtgaatgggaaaacatgtttttcctcctgtcctaaaatagaacctgatgacgggagggagcatcagtcagtctggcctaaatcagtgtactgcgcttttggatgcgggcaaaagtattgtcatgttgattactttataataacgtattaatatcattggctcaaacagttgtgatatatacctagtaatcatcgaggatctcaaagatatgcaccagtttgaataaatgctgtttaaagattaaattttacaattcattttaaattgtcattttgtgacaatatttctaaacaacgtccatttttaataaca

SEQ ID NO: 70 - protein, 858 aa, Homo sapiens; identical to SEQ ID NO: 26

SEQ ID NO: 71 - DNA, 1061 bp, Homo sapiens
  CDS (183)..(908): NM_002506 WT, CDS 183-908
gctccggcacagcagagagcgctgggagccggaggggagcgcagcgagttttggccagtggtcgtgcagtccaaggggctggatggcatgctggacccaagctcagctcagcgtccggacccaataacagttttaccaagggagcagctttctatcctggccacactgaggtgcatagcgtaatgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcctgacctgccgacacgctccctccccctgccccttctacactctcctgggcccctccctacctcaacctgtaaattattttaaattataaggactgcatggtaatttatagtttatacagttttaaagaatcattatttattaaatttttggaagca

SEQ ID NO: 72 - protein, 241 aa, Homo sapiens; identical to SEQ ID NO: 32

SEQ ID NO: 73 - DNA, 3728 bp, Homo sapiens
  CDS (1020)..(1745): NM_002608 WT, CDS 1020-1745
ggcaacttctcctcctgcagccgggagcggcctgcctgcctccctgcgcacccgcagcctcccccgctgcctccctagggctcccctccggccgccagcgcccatttttcattccctagatagagatactttgcgcgcacacacatacatacgcgcgcaaaaaggaaaaaaaaaaaaaaaagcccaccctccagcctcgctgcaaagagaaaaccggagcagccgcagctcgcagctcgcagctcgcagcccgcagcccgcagaggacgcccagagcggcgagcgggcgggcagacggaccgacggactcgcgccgcgtccacctgtcggccgggcccagccgagcgcgcagcgggcacgccgcgcgcgcggagcagccgtgcccgccgcccgggccccgcgccagggcgcacacgctcccgcccccctacccggcccgggcgggagtttgcacctctccctgcccgggtgctcgagctgccgttgcaaagccaactttggaaaaagttttttgggggagacttgggccttgaggtgcccagctccgcgctttccgattttgggggcctttccagaaaatgttgcaaaaaagctaagccggcgggcagaggaaaacgcctgtagccggcgagtgaagacgaaccatcgactgccgtgttccttttcctcttggaggttggagtcccctgggcgcccccacacggctagacgcctcggctggttcgcgacgcagccccccggccgtggatgctcactcgggctcgggatccgcccaggtagcggcctcggacccaggtcctgcgcccaggtcctcccctgccccccagcgacggagccggggccgggggcggcggcgcccgggggccatgcgggtgagccgcggctgcagaggcctgagcgcctgatcgccgcggacccgagccgagcccacccccctccccagccccccaccctggccgcgggggcggcgcgctcgatctacgcgtccggggccccgcggggccgggcccggagtcggcatgaatcgctgctgggcgctcttcctgtctctctgctgctacctgcgtctggtcagcgccgagggggaccccattcccgaggagctttatgagatgctgagtgaccactcgatccgctcctttgatgatctccaacgcctgctgcacggagaccccggagaggaagatggggccgagttggacctgaacatgacccgctcccactctggaggcgagctggagagcttggctcgtggaagaaggagcctgggttccctgaccattgctgagccggccatgatcgccgagtgcaagacgcgcaccgaggtgttcgagatctcccggcgcctcatagaccgcaccaacgccaacttcctggtgtggccgccctgtgtggaggtgcagcgctgctccggctgctgcaacaaccgcaacgtgcagtgccgccccacccaggtgcagctgcgacctgtccaggtgagaaagatcgagattgtgcggaagaagccaatctttaagaaggccacggtgacgctggaagaccacctggcatgcaagtgtgagacagtggcagctgcacggcctgtgacccgaagcccggggggttcccaggagcagcgagccaaaacgccccaaactcgggtgaccattcggacggtgcgagtccgccggccccccaagggcaagcaccggaaattcaagcacacgcatgacaagacggcactgaaggagacccttggagcctaggggcatcggcaggagagtgtgtgggcagggttatttaatatggtatttgctgtattgcccccatggggtccttggagtgataatattgtttccctcgtccgtctgtctcgatgcctgattcggacggccaatggtgcttcccccacccctccacgtgtccgtccacccttccatcagcgggtctcctcccagcggcctccggcgtcttgcccagcagctcaagaagaaaaagaaggactgaactccatcgccatcttcttcccttaactccaagaacttgggataagagtgtgagagagactgatggggtcgctctttgggggaaacgggctccttcccctgcacctggcctgggccacacctgagcgctgtggactgtcctgaggagccctgaggacctctcagcatagcctgcctgatccctgaacccctggccagctctgaggggaggcacctccaggcaggccaggctgcctcggactccatggctaagaccacagacgggcacacagactggagaaaacccctcccacggtgcccaaacaccagtcacctcgtctccctggtgcctctgtgcacagtggcttcttttcgttttcgttttgaagacgtggactcctcttggtgggtgtggccagcacaccaagtggctgggtgccctctcaggtgggttagagatggagtttgctgttgaggtggctgtagatggtgacctgggtatcccctgcctcctgccaccccttcctccccacactccactctgattcacctcttcctctggttcctttcatctctctacctccaccctgcattttcctcttgtcctggcccttcagtctgctccaccaaggggctcttgaaccccttattaaggccccagatgatcccagtcactcctctctagggcagaagactagaggccagggcagcaagggacctgctcatcatattccaacccagccacgactgccatgtaaggttgtgcagggtgtgtactgcacaaggacattgtatgcagggagcactgttcacatcatagataaagctgatttgtatatttattatgacaatttctggcagatgtaggtaaagaggaaaaggatccttttcctaattcacacaaagactccttgtggactggctgtgcccctgatgcagcctgtggcttggagtggccaaataggagggagactgtggtaggggcagggaggcaacactgctgtccacatgacctccatttcccaaagtcctctgctccagcaactgcccttccaggtgggtgtgggacacctgggagaaggtctccaagggagggtgcagccctcttgcccgcacccctccctgcttgcacacttccccatctttgatccttctgagctccacctctggtggctcctcctaggaaaccagctcgtgggctgggaatgggggagagaagggaaaagatccccaagaccccctggggtgggatctgagctcccacctcccttcccacctactgcactttcccccttcccgccttccaaaacctgcttccttcagtttgtaaagtcggtgattatatttttgggggctttccttttattttttaaatgtaaaatttatttatattccgtatttaaagttgtaaaaaaaaataaccacaaaacaaaaccaaatgaatccgccggaggtctgtctgttggcatcgtgcgtgacaattaacctttctgccttggcaggatgtgccgacagcttgcggcgtgttcctctcactctgggagcctcaggcgtgatctcacacactggcgtgcacatacacacacacacacatacatgctcacacatgcgtgcacatacacgcaggcctgcaacttgggggaggcctctgtctggcgggaagaagagacacacaggctactctgttggtcttggtcctggcacagctcctgacacgtggacttgtgcgtgtctctggcagtgacgagagatgggtttctgcag

SEQ ID NO: 74 - protein, 241 aa, Homo sapiens; identical to SEQ ID NO: 34

SEQ ID NO: 75 - DNA, 813 bp, Homo sapiens
  CDS (72)..(563): NM_052872 WT, CDS 72-563
gaacacaggcatacacaggaagatacattcacagaaagagcttcctgcacaaagtaagccaccagcgcaacatgacagtgaagaccctgcatggcccagccatggtcaagtacttgctgctgtcgatattggggcttgcctttctgagtgaggcggcagctcggaaaatccccaaagtaggacatacttttttccaaaagcctgagagttgcccgcctgtgccaggaggtagtatgaagcttgacattggcatcatcaatgaaaaccagcgcgtttccatgtcacgtaacatcgagagccgctccacctccccctggaattacactgtcacttgggaccccaaccggtacccctcggaagttgtacaggcccagtgtaggaacttgggctgcatcaatgctcaaggaaaggaagacatctccatgaattccgttcccatccagcaagagaccctggtcgtccggaggaagcaccaaggctgctctgtttctttccagttggagaaggtgctggtgactgttggctgcacctgcgtcacccctgtcatccaccatgtgcagtaagaggtgcatatccactcagctgaagaagctgtagaaatgccactccttacccagtgctctgcaacaagtcctgtctgacccccaattccctccacttcacaggactcttaataagacctgcacggatggaaacagaaaatattcacaatgtatgtgtgtatgtactacactttatatttgatatctaaaatgttaggagaaaaattaatatattcagtgctaatataataaagtattaataatttaaaaa

SEQ ID NO: 76 - protein, 163 aa, Homo sapiens; identical to SEQ ID NO: 36

SEQ ID NO: 77 - DNA, 4171 bp, Homo sapiens
  CDS (562)..(1197): NM_000514 WT, CDS 562-1197
agcctgcgctcctggcgccctcatgtcttcacgggactccccgcgccggttgacgtggtgtctcgttcggatctccaggcaagacctcagctccggcagcagcatcagacaaaccagtctcgtgctcccaggcagtgcgcccagaggaggcgcagagcgcggcagctgccgctgagccgcccgcagcgccccgggcccgcgcagccccagccaagagcgcgacgcgcgcagccctgtcagccccccacccaaagcagcggcggctgctcggacctcggcttctgggggtgcgggggcccggcgggagagttgccggcagccctcgccctgttggcggcggcggcggcgggagtcttggccgccgcctccagcgcgcccttgctgccccgcgcgaccccaggattgcgaactcttgcccctgacctgttgggcggggctccgcgctccagccatcagcccggatgggtctcctggctgggacttggggcacctggagttaatgtccaacctagggtctgcggagacccgatccgaggtgccgccgccggacgggactttaagatgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctgactccggctccagagactgctgtgtattgcattcctgctacagtgcaaagaaagggaccaaggttcccaggaaatgtttgcccagaatggaagatgaggaccaaggaggcggaggaggaggaagaagaagaggaggaggaggaggaggaggaggaggaggaggaaggcagccatcatgggagcctggtagagggagatccagctacagacaactggacaggagagagagaaaacagccctctggattctccaggatggcagccgatgtcactagaagctcagggctgatgttcctggttggctattgccaccatttcagctgatacagtccaccatcactgattaccggcgcggttgcggtggatgcacttgaaccaaaccagtgtatctcctgtgatttgttttcatgtgtccgaagacaccagggaaacagagatcctggtgttgttccttgttattacgttttactgctgaaagtaagaggtttatttttctgtcactcagtggagacataccctggaaaggagaggggaaaaaaaaagcaaagatacaagagataatcacctttgcatttgaaagttgaggcccgaggtttactacaaccagcatttttgccaacggttggtgattgatttccatcacggtgtgtggggtgggaagaagttggctaggaaccaaaaaggctgtgctcatgattaaacacaaacctgaaggtatttcctttatgtccttggaaacaggaaacgagttgtggttttcgccagcattcttgtaggagagaatcggggaaggccccgaactgcccccgggcagggagagcccctcaggcctgttggtttacagagagacagatgttacataaccagctccgttgatgcgtggtcaccagtgaccagagaagctactcgatgcaatgcatctgtttcagatacagaaatatagagaagatatttattgaaatttaagttattgttatttattaccgttcactaatgaatttctcttttttcccttatttattaaagtttcttttcaaaggtgccaaagtatatgtgctcgcaaaatgcaaagaaaggtgacaaaaggaaatttgaattgggaacaagggtccatgcttttcaaagtattaaaaagttttttgccaggcaaaaatcacttactttacctttttaagaaaatttgtcattaattttccccagatttcagcatttttcccaatttttatttgtggagcatctcaggcaagccccctttcctggagcagcgtgcagagaccactggcacttgactttatttcttccttgctccattgctgaacagaaatgtcgtgggctccacttcctgttgtctttaagctcttagtcccctccacgtatacctatctgtactatgcataaccatatgtagaaaaggttcagttccttttagtaggtagtcctggatttaatgctgacctaaaagtaatgtcgacaatgctgtcaggtagctgccgttctaccgactccctccatccctgcccacccactgccctcccgagaatatgctggctgcccagtgcagcccgggagacacaggggccttccagaggtagggtctaccaggtcctgtacaacccctgggctgtcaccgggggtcaacagctgctgctcctatatacccaaacacctgacagctccctggggagcagatggctgagaagggtgctgaggaagccatattgggaccagccacagccacacacatggagcctcatacttaggagcgtgctgcctttaaatgaaggtggtcggggccagtgcagcggctcacacccataatcccaacactttggaaagccaaggtgggaggatctcttgaacccaggagtttgagaccagcttgggcaacatagggagaccctgtctctacagaaactttaaaaattaggcaggcatgatggtgcacacctgtggtcccagctactcaagaggctgaaggaggatcacttgagtccagaaggtcgaggctgcagtgagctgtgatcatgccactgcactccagcctaagtgacagtgcggtaccctgtctcaaaaaaaaaaaaaaaaaaaaaaagaggttggagcaggaggaagcataggggcgggaacagccacctcctccatgccctagattgtgaatttatcgggcagccaacacatgtatgacacactaggccctgtattacagcttgttacgcatttcataaaagggattttcattaaggagataatctattactacctaccttagtggctactagtataaaactatgacagatttagcaattaaatgaaatactggcctccatcaaataatcatagtaacaagaagcagcagttaccagacatctgatccccttcccccaaaatacccaaattcttcatggttctgcccttctctgtcctttctgctccccttgctcgcctgggaaatggaggaaaggccttccctctcacactgtcttgggatcttgctgagaattcagactgctcgaaacagtgacaaaccccagccatccagtcattcgtggagcacaatttggatgtggccccaggggcatctgtcccattcagagaaccttggcagtgcgatggccactgttcccaggcttcaacctcagtgaccccccccaacaactccccatggagagtccctgcccaaaaaagctgtaggatccaaggggtgtcaatagctcgttcccggcatcacctacacaccacaagcaggttttaatggaagcaagttgctccaccaaatccacaaaagggtaaagtttgtgatttttctttatcattgcgatcaccatctgataccgtaaggagtgcacttgtttggaagttctgacttctctgatctgtcttggtcgtttgtgttataaaaccaaagttctctacagactttatttttgtacaatatcattttgtaactttttacaaataaaaactcatttctattgc

SEQ ID NO: 78 - protein, 211 aa, Homo sapiens; identical to SEQ ID NO: 38

SEQ ID NO: 79 - DNA, 1168 bp, Homo sapiens
  CDS (84)..(857): NM_002527 WT, CDS 84-857
tgccagaataacacagactcagctgccagagcctgctcttaacacctgtgtttccttttcagatcttacaggtgaacaaggtgatgtccatcttgttttatgtgatatttctcgcttatctccgtggcatccaaggtaacaacatggatcaaaggagtttgccagaagactcgctcaattccctcattattaagctgatccaggcagatattttgaaaaacaagctctccaagcagatggtggacgttaaggaaaattaccagagcaccctgcccaaagctgaggctccccgagagccggagcggggagggcccgccaagtcagcattccagccggtgattgcaatggacaccgaactgctgcgacaacagagacgctacaactcaccgcgggtcctgctgagcgacagcacccccttggagcccccgcccttgtatctcatggaggattacgtgggcagccccgtggtggcgaacagaacatcacggcggaaacggtacgcggagcataagagtcaccgaggggagtactcggtatgtgacagtgagagtctgtgggtgaccgacaagtcatcggccatcgacattcggggacaccaggtcacggtgctgggggagatcaaaacgggcaactctcccgtcaaacaatatttttatgaaacgcgatgtaaggaagccaggccggtcaaaaacggttgcaggggtattgatgataaacactggaactctcagtgcaaaacatcccaaacctacgtccgagcactgacttcagagaacaataaactcgtgggctggcggtggatacggatagacacgtcctgtgtgtgtgccttgtcgagaaaaatcggaagaacatgaattggcatctctccccatatataaattattactttaaattatatgatatgcatgtagcatataaatgtttatattgtttttatatattataagttgacctttatttattaaacttcagcaaccctacagtatataagcttttttctcaataaaatcagtgtgcttgccttccctcaggcctctcccatctgttaaaacttgttttgtgatccggctctcaggagtcactctgtaaaatctgtgtacaccagtattttgcattcagtattgtcaaggccatgactgttgttttagtaaacttgttaaaatca

SEQ ID NO: 80 - protein, 257 aa, Homo sapiens; identical to SEQ ID NO: 40

SEQ ID NO: 81 - DNA, 1211 bp, Homo sapiens
  CDS (126)..(626): NM_000619 WT, CDS 126-626
acattgttctgatcatctgaagatcagctattagaagagaaagatcagttaagtcctttggacctgatcagcttgatacaagaactactgatttcaacttctttggcttaattctctcggaaacgatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaatggttgtcctgcctgcaatatttgaattttaaatctaaatctatttattaatatttaacattatttatatggggaatatatttttagactcatcaatcaaataagtatttataatagcaacttttgtgtaatgaaaatgaatatctattaatatatgtattatttataattcctatatcctgtgactgtctcacttaatcctttgttttctgactaattaggcaaggctatgtgattacaaggctttatctcaggggccaactaggcagccaacctaagcaagatcccatgggttgtgtgtttatttcacttgatgatacaatgaacacttataagtgaagtgatactatccagttactgccggtttgaaaatatgcctgcaatctgagccagtgctttaatggcatgtcagacagaacttgaatgtgtcaggtgaccctgatgaaaacatagcatctcaggagatttcatgcctggtgcttccaaatattgttgacaactgtgactgtacccaaatggaaagtaactcatttgttaaaattatcaatatctaatatatatgaataaagtgtaagttcacaacta

SEQ ID NO: 82 - protein, 166 aa, Homo sapiens; identical to SEQ ID NO: 42

SEQ ID NO: 83 - DNA, 4766 bp, Homo sapiens
  CDS (1095)..(1838): NM_170735.6 WT, CDS 1095-1838
gcgcgcgcgcacacacacacacacacacagagagaacatctctagtaaaaagaaaagttgagctttcttagctagatgtgtgtattagccagaaaaagccaaggagtgaagggttttagagaactggaggagataaagtggagtctgcatatgggaggcatttgaaatggacttaaatgtctttttaatgctgactttttcagttttctccttaccagacacattgttttcatgacattagccccaggcatagacacatcattaaaatgaacatgtcaaaaaatgatttctgtttagaaataagcaaaacattttcagttgtgaccacccaggtgtagaataaagaacagtggaattgggagccctgagttctaacataaactttcttcatgacataaggcaagtcttctatggcctttggtttccttacctgtaaaacaggatggctcaatgaaattatctttcttctttgctataatagagtatctctgtgggaagaggaaaaaaaaagtcaatttaaaggctccttatagttccccaactgctgttttattgtgctattcatgcctagacatcacatagctagaaaggcccatcagacccctcaggccactgctgttcctgtcacacattcctgcaaaggaccatgttgctaacttgaaaaaaattactattaattacacttgcagttgttgcttagtaacatttatgattttgtgtttctcgtgacagcatgagcagagatcattaaaaattaaacttacaaagctgctaaagtgggaagaaggagaacttgaagccacaatttttgcacttgcttagaagccatctaatctcaggtttatatgctagatcttgggggaaacactgcatgtctctggtttatattaaaccacatacagcacactactgacactgatttgtgtctggtgcagctggagtttatcaccaagacataaaaaaaccttgaccctgcagaatggcctggaattacaatcagatgggccacatggcatcccggtgaaagaaagccctaaccagttttctgtcttgtttctgctttctccctacagttccaccaggtgagaagagtgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagtggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaagttaaaaaaaataataaattgcatgctgctttaattgtgaattgataataaactgtcctctttcagaaaacagaaaaaaaacacacacacacacaacaaaaatttgaaccaaaacattccgtttacattttagacagtaagtatcttcgttcttgttagtactatatctgttttactgcttttaacttctgatagcgttggaattaaaacaatgtcaaggtgctgttgtcattgctttactggcttaggggatgggggatggggggtatatttttgtttgttttgtgtttttttttcgtttgtttgttttgttttttagttcccacagggagtagagatggggaaagaattcctacaatatatattctggctgataaaagatacatttgtatgttgtgaagatgtttgcaatatcgatcagatgactagaaagtgaataaaaattaaggcaactgaacaaaaaaatgctcacactccacatcccgtgatgcacctcccaggccccgctcattctttgggcgttggtcagagtaagctgcttttgacggaaggacctatgtttgctcagaacacattctttccccccctccccctctggtctcctctttgttttgttttaaggaagaaaaatcagttgcgcgttctgaaatattttaccactgctgtgaacaagtgaacacattgtgtcacatcatgacactcgtataagcatggagaacagtgatttttttttagaacagaaaacaacaaaaaataaccccaaaatgaagattattttttatgaggagtgaacatttgggtaaatcatggctaagcttaaaaaaaactcatggtgaggcttaacaatgtcttgtaagcaaaaggtagagccctgtatcaacccagaaacacctagatcagaacaggaatccacattgccagtgacatgagactgaacagccaaatggaggctatgtggagttggcattgcatttaccggcagtgcgggaggaatttctgagtggccatcccaaggtctaggtggaggtggggcatggtatttgagacattccaaaacgaaggcctctgaaggacccttcagaggtggctctggaatgacatgtgtcaagctgcttggacctcgtgctttaagtgcctacattatctaactgtgctcaagaggttctcgactggaggaccacactcaagccgacttatgcccaccatcccacctctggataattttgcataaaattggattagcctggagcaggttgggagccaaatgtggcatttgtgatcatgagattgatgcaatgagatagaagatgtttgctacctgaacacttattgctttgaaactagacttgaggaaaccagggtttatcttttgagaacttttggtaagggaaaagggaacaggaaaagaaaccccaaactcaggccgaatgatcaaggggacccataggaaatcttgtccagagacaagacttcgggaaggtgtctggacattcagaacaccaagacttgaaggtgccttgctcaatggaagaggccaggacagagctgacaaaattttgctccccagtgaaggccacagcaaccttctgcccatcctgtctgttcatggagagggtccctgcctcacctctgccattttgggttaggagaagtcaagttgggagcctgaaatagtggttcttggaaaaatggatccccagtgaaaactagagctctaagcccattcagcccatttcacacctgaaaatgttagtgatcaccacttggaccagcatccttaagtatcagaaagccccaagcaattgctgcatcttagtagggtgagggataagcaaaagaggatgttcaccataacccaggaatgaagataccatcagcaaagaatttcaatttgttcagtctttcatttagagctagtctttcacagtaccatctgaatacctctttgaaagaaggaagactttacgtagtgtagatttgttttgtgttgtttgaaaatattatctttgtaattatttttaatatgtaaggaatgcttggaatatctgctgtatgtcaactttatgcagcttccttttgagggacaaatttaaaacaaacaaccccccatcacaaacttaaaggattgcaagggccagatctgttaagtggtttcataggagacacatccagcaattgtgtggtcagtggctcttttacccaataagatacatcacagtcacatgcttgatggtttatgttgacctaagatttattttgttaaaatctctctctgttgtgttcgttcttgttctgttttgttttgttttttaaagtcttgctgtggtctctttgtggcagaagtgtttcatgcatggcagcaggcctgttgcttttttatggcgattcccattgaaaatgtaagtaaatgtctgtggccttgttctctctatggtaaagatattattcaccatgtaaaacaaaaaacaatatttattgtattttagtatatttatataattatgttattgaaaaaaattggcattaaaacttaaccgcatcagaagcctattgtaaatacaagttctatttaagtgtactaattaacatataatatatgttttaaatatagaatttttaatgtttttaaatatattttcaaagtacataaaa

SEQ ID NO: 84 - protein, 247 aa, Homo sapiens; identical to SEQ ID NO: 44

SEQ ID NO: 85 - DNA, 1527 bp, Artificial Sequence
  codon-optimized gene of BDNF Fc fusion
  CDS (13)..(1521)
aagcttgccaccatgaccatcctgttcctgacaatggtcatctcctactttggctgcatgaaggccgctcctatgaaggaggctaacatcagaggacagggaggactggcttatccaggcgtgcgcacccacggcacactggagagcgtgaatggaccaaaggctggaagcagaggactgacctctctggctgacacattcgagcatgtgatcgaggagctgctggacgaggatcagaaggtgagaccaaacgaggagaacaataaggacgccgatctgtacacctctcgcgtgatgctgtccagccaggtgccactggagccacctctgctgtttctgctggaggagtacaagaactatctggatgccgctaatatgtccatgcgcgtgaggcggcacagcgacccagctagaaggggagagctgtccgtgtgcgattctatctccgagtgggtgaccgccgctgacaagaagacagctgtggatatgagcggcggcaccgtgacagtgctggagaaggtgcccgtgtctaagggccagctgaagcagtacttctatgagaccaagtgcaaccctatgggctacacaaaggagggctgtaggggcatcgacaagcggcattggaactcccagtgtagaaccacacagtcctatgtgcgcgctctgaccatggacagcaagaagaggatcggctggaggttcatccgcatcgatacatcttgcgtgtgcaccctgacaatcaagaggggaaggggaggaggaggatccggaggaggcggcagtggcggcggcggctctggcggcggcggctccggcggcggcggctccgtgaggagcgacaagacccacacatgcccaccatgtcctgctccagagctgctgggaggacctagcgtgttcctgtttcctccaaagccaaaggataccctgatgatctcccggacccctgaggtgacatgcgtggtggtggacgtgagccacgaggatccagaggtgaagttcaactggtacgtggacggcgtggaggtgcataatgctaagaccaagcccagggaggagcagtacaatagcacctatcgggtggtgtctgtgctgacagtgctgcatcaggattggctgaacggcaaggagtataagtgcaaggtgtctaataaggccctgcccgctcctatcgagaagaccatctccaaggctaagggacagccaagggagccacaggtgtacacactgcccccttctcgggaggagatgaccaagaaccaggtgtccctgacatgtctggtgaagggcttctatccctccgacatcgctgtggagtgggagagcaatggccagcctgagaacaattacaagaccacaccacccgtgctggactctgatggctccttctttctgtatagcaagctgaccgtggataagtctcggtggcagcagggcaacgtgtttagctgttctgtgatgcacgaggccctgcacaatcattacacacagaagtccctgagcctgtctcctggcaagtgagaattc

SEQ ID NO: 86 - protein, 502 aa, Artificial Sequence
  Synthetic Construct
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGRGGGGSGGGGSGGGGSGGGGSGGGGSVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 5 aa, Artificial Sequence
  GS linker
GGGGS

SEQ ID NO: 88 - protein, 6 aa, Artificial Sequence
  H4 linker
EAAAAK

SEQ ID NO: 89 - DNA, 687 bp, Artificial Sequence
  codon-optimized gene of BDNF precursor
  CDS (1)..(687)
gccccaatgaaggaggctaacatcaggggacagggaggactggcttaccctggcgtgcggacccacggcacactggagtccgtgaatggcccaaaggctggctcccggggcctgaccagcctggctgacacattcgagcatgtgatcgaggagctgctggacgaggatcagaaggtgagacctaacgaggagaacaataaggacgctgatctgtataccagccgcgtgatgctgtccagccaggtgccactggagccacctctgctgtttctgctggaggagtacaagaactatctggatgccgctaatatgtccatgagggtgaggcggcacagcgacccagctagaaggggagagctgtccgtgtgcgattccatcagcgagtgggtgaccgccgctgacaagaagacagctgtggatatgagcggcggcaccgtgacagtgctggagaaggtgcccgtgtctaagggccagctgaagcagtatttctacgagaccaagtgcaaccctatgggctacacaaaggagggctgtaggggcatcgacaagcggcattggaactcccagtgtagaaccacacagtcctatgtgcgcgccctgaccatggactccaagaagaggatcggctggaggttcatccgcatcgatacaagctgcgtgtgcaccctgacaatcaagaggggccgg

SEQ ID NO: 90 - protein, 229 aa, Artificial Sequence
  Synthetic Construct
APMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 91 - DNA, 357 bp, Artificial Sequence
  mature BDNF WT
  CDS (1)..(357)
cacagcgacccagctagaaggggagagctgtccgtgtgcgattccatcagcgagtgggtgaccgccgctgacaagaagacagctgtggatatgagcggcggcaccgtgacagtgctggagaaggtgcccgtgtctaagggccagctgaagcagtatttctacgagaccaagtgcaaccctatgggctacacaaaggagggctgtaggggcatcgacaagcggcattggaactcccagtgtagaaccacacagtcctatgtgcgcgccctgaccatggactccaagaagaggatcggctggaggttcatccgcatcgatacaagctgcgtgtgcaccctgacaatcaagaggggccgg

SEQ ID NO: 92 - protein, 119 aa, Artificial Sequence
  Synthetic Construct
HSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 93 - DNA, 57 bp, Artificial Sequence
  IgG signal sequence 6SVL_B
  CDS (1)..(57)
atgggctggtctctgatcctgctgttcctggtggctgtggctaccagggtgggatcc

SEQ ID NO: 94 - protein, 19 aa, Artificial Sequence
  Synthetic Construct
MGWSLILLFLVAVATRVGS

SEQ ID NO: 95 - DNA, 696 bp, Homo sapiens
  CDS (1)..(696): Human Fc fragment JN222933
gagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 96 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 20 aa, Artificial Sequence
  Human IL2 signal peptide
MYRMQLLSCIALSLALVTNS

SEQ ID NO: 98 - protein, 17 aa, Artificial Sequence
  Human albumin signal peptide
MKWVTFISLFLFSSAYS

SEQ ID NO: 99 - DNA, 1419 bp, Artificial Sequence
  codon-optimized gene of GDNF Fc fusion
  CDS (13)..(1413)
aagcttgccaccatgaagctgtgggatgtggtggccgtgtgcctggtgctgctgcacaccgcttccgccttccctctgccagctggcaagaggccacctgaggctcccgctgaggatagaagcctgggcaggcggagagccccttttgctctgtccagcgactctaacatgcccgaggattaccctgaccagttcgacgatgtgatggacttcatccaggccacaatcaagcgcctgaagaggtctccagacaagcagatggctgtgctgccaaggagggagaggaatagacaggctgctgctgctaacccagagaactccaggggcaagggccggagaggacagaggggcaagaacagaggctgcgtgctgaccgccatccatctgaatgtgacagatctgggcctgggctacgagaccaaggaggagctgatcttcaggtattgctctggctcctgtgatgccgctgagaccacatacgacaagatcctgaagaacctgagccgcaatcgcaggctggtgtctgacaaagtgggccaggcctgctgtaggcctatcgctttcgacgatgacctgtcctttctggatgacaacctggtgtatcacatcctgcgcaagcatagcgccaagaggtgcggctgcatcggaggaggaggaagcggaggaggaggatctggcggcggcggctctggcggcggcggcagcggcggcggcggctccgtgcggagcgataagacccacacatgcccaccatgtccagctcctgagctgctgggaggaccttccgtgttcctgtttcctccaaagccaaaggacaccctgatgatctccagaacccctgaggtgacatgcgtggtggtggatgtgagccacgaggacccagaggtgaagtttaactggtacgtggatggcgtggaggtgcataatgccaagacaaagccacgcgaggagcagtacaattctacctatagggtggtgtccgtgctgacagtgctgcatcaggactggctgaacggcaaggagtataagtgcaaggtgtctaataaggccctgccagctcccatcgagaagaccatctccaaggctaagggacagcctagggagccacaggtgtacacactgccccctagcagagaggagatgaccaagaaccaggtgtctctgacatgtctggtgaagggcttctatccatctgatatcgctgtggagtgggagtccaatggccagcccgagaacaattacaagaccacaccacccgtgctggattctgacggctccttctttctgtattccaagctgaccgtggacaagagcagatggcagcagggcaacgtgtttagctgttctgtgatgcacgaggctctgcacaatcattatacacagaagtccctgagcctgtctcctggcaagtgagaattc

SEQ ID NO: 100 - protein, 466 aa, Artificial Sequence
  Synthetic Construct
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCIGGGGSGGGGSGGGGSGGGGSGGGGSVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK